(19) United States
(12) Patent Application Publication
(10) Pub. No.: US 2025/0325715 A1
(43) Pub. Date: Oct. 23, 2025

(54) RADIOLABELED LIPOSOMES AND METHODS OF USE FOR TREATING LEPTOMENINGEAL METASTASES

(71) Applicants: Plus Therapeutics, Inc., Austin, TX (US); NanoTx, Corp., San Antonio, TX (US)

(72) Inventors: Marc Hedrick, Charlottesville, VA (US); Cheryl D. Rice, San Diego, CA (US); Daniel Ortega, San Antonio, TX (US); Andrew Brenner, Austin, TX (US); Gregory D. Stein, Austin, TX (US); Ande Bao, San Antonio, TX (US); William Phillips, Austin, TX (US)

(73) Assignees: Plus Therapeutics, Inc., Austin, TX (US); NanoTX, Corp., San Antonio, TX (US)

(21) Appl. No.: 18/784,347

(22) Filed: Jul. 25, 2024

Related U.S. Application Data
(63) Continuation of application No. PCT/US2023/011564, filed on Jan. 25, 2023.
(60) Provisional application No. 63/302,953, filed on Jan. 25, 2022.
(60) Provisional application No. 63/333,050, filed on Apr. 20, 2022.
(60) Provisional application No. 63/343,034, filed on May 17, 2022.

(51) Int. Cl. A61K 51/04; A61K 9/00; A61K 45/06; A61P 35/04

(52) U.S. Cl. CPC A61K 51/0408; A61K 9/0019; A61K 45/06; A61K 51/0497; A61P 35/04; A61K 2121/00; A61K 2123/00

(57) ABSTRACT

Radiolabeled liposomes can be used in the treatment of cancer. These local therapies can be used to treat cancers including, but not limited to, leptomeningeal metastases. In an embodiment, radiolabeled liposomes comprising (186Re) obisbemeda can be administered to a patient through an Ommaya reservoir. The therapies can include imaging the radiolabeled liposome concomitant or subsequent to administration.

### CROSS-REFERENCE

This application claims the benefit of U.S. Provisional Application No. 63/302,953, filed Jan. 25, 2022; U.S. Provisional Application No. 63/333,050, filed Apr. 20, 2022; and U.S. Provisional Application No. 63/343,034, filed May 17, 2022, all of which are incorporated herein by reference.

### BACKGROUND OF THE INVENTION

Glioblastoma (GBM) is the most common and aggressive primary malignant brain tumor in adults. The standard treatment for GBM over the last decade has been surgery followed by concomitant chemoradiotherapy with temozolomide, with radiation being a major contributor to survival in this regimen. While external beam radiation therapy (EBRT) remains a central component of the management of primary brain tumors, it is limited by tolerance of the surrounding normal brain tissue. As most recurrences occur within two centimeters of the resection margin, there remains an urgent need for the development of local therapies, such as brachytherapy.

Leptomeningeal metastases (LM), also known as leptomeningeal carcinomatosis, is an uncommon but typically fatal complication of many advanced cancers in which cancer cells metastasize. Although any solid tumor has the potential for LM, most commonly LM originates from four common primary cancers, breast, lung, melanoma and gastrointestinal which metastasize to the central nervous system (CNS) and are found in either the leptomeninges (membrane surrounding the brain and spinal cord) or cerebrospinal fluid (CSF) (circulates nutrients and chemicals to the brain and spinal cord). It often goes undiagnosed due to a lack of symptoms, and causes neurological complications such as difficulty thinking, double vision, and headaches. LM are diagnosed in ˜5% of patients with metastatic cancer. LM occurs in 5-8% of patients with solid tumors. There are no good treatments in generally recognized standards of care, with patients often having to choose between toxic treatments and a very short life expectancy.

Most solid tumors are known to cause LM, but the most common solid tumors giving rise to LM are breast cancer, lung cancer, melanoma, gastrointestinal and primary central nervous system tumors. Other LM categories include hematologic malignancy (leukemia and lymphoma) and primary CNS tumors (notably medulloblastoma). Standard treatment includes radiation therapy (RT) to the affected sites followed by chemotherapy delivered into the cerebrospinal fluid (CSF) or systemic treatment of the underlying malignancy. Most intrathecal and systemic chemotherapy have difficult side effects. The median survival depends on the primary tumor source and is usually 2-4 months. If untreated, survival is usually around 6-8 weeks. Neurological symptoms are usually fixed and rarely improve with treatment. Novel treatment approaches that can prolong survival and maintain quality of the life by delaying further neurological deterioration are desperately needed for LM patients. Treatment of LM with external beam radiation must travel through and therefore harm surrounding normal tissues to get to the tumors. The risk of significant side effects from entire neuroaxis radiation therapy generally outweighs the benefits in this relatively radioresistant tumor. Focal radiation therapy relieves neurological symptoms but has no significant effect on survival. Other radiotherapeutics typically cannot reach and destroy the tumor through systemic administration because of the blood-brain barrier.

Thalamic tumors comprise approximately 5% of brain tumors. Thalamic tumors are difficult to surgically remove due to their location. Selected examples of thalamic tumors are gliomas such as astrocytomas and glioblastomas.

Brain stem tumors are difficult to surgically remove due to location and can effect basic functions such as breathing and heartbeat. Selected examples of brain stem tumors are diffuse intrinsic brain stem glioma (DIPG) and focal brain stem glioma.

There remains a need to develop local therapies for cancers in general, such as lung cancer, breast cancer, colorectal cancer, prostate cancer, skin cancer, stomach cancer, bladder cancer, liver cancer, leukemia, lymphoma, ovarian cancer, pancreatic cancer, hepatocellular carcinoma, melanoma, sarcoma, head and neck cancer, thalamic tumors, brain stem tumors, glioma, glioblastoma, medulloblastoma, ependymoma, diffuse intrinsic pontine glioma, leptomeningeal metastases, and pediatric high-grade glioma.

### SUMMARY OF THE INVENTION

Provided herein, in one aspect, is a method of treating a disease or disorder in a subject in need thereof, comprising administering a therapeutically effective amount of a radiolabeled liposome comprising a liposome and a compound of Formula I:

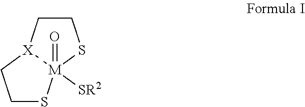

or a pharmaceutically acceptable salt thereof, wherein:
   M is ^{99m}Tc, ¹⁸⁶Re, ¹⁸⁸Re, or a combination thereof;
   X is NR¹;
   R¹ is CH₂CH₂NEt₂ or CH₂CH₂CH₂CH₃; and
   R² is CH₂CH₂N(CH₂CH₂SH)(CH₂CH₂NEt₂) or
CH₂CH₂N(CH₂CH₂SH)(CH₂CH₂CH₂CH₃).

In some embodiments, R¹ is CH₂CH₂NEt₂ and R² is CH₂CH₂N(CH₂CH₂SH)(CH₂CH₂NEt₂). In some embodiments, R¹ is CH₂CH₂CH₂CH₃ and R² is CH₂CH₂N(CH₂CH₂SH)(CH₂CH₂CH₂CH₃). In some embodiments, M is ¹⁸⁶Re.

In some embodiments, the compound is incorporated or attached to the liposome.

In some embodiments, the liposome further comprises a drug that is incorporated within the liposome.

In some embodiments, the drug is a compound comprising at least one thiol group. In some embodiments, the drug reacts with the compound. In some embodiments, the drug comprises glutathione, cysteine, N-acetyl cysteine, 2-mercaptosuccinic acid, 2,3-dimercaptosuccinic acid, captopril, or a combination thereof.

In some embodiments, the liposome comprises a lipid. In some embodiments, the liposome comprises a phospholipid.

In some embodiments, the liposome comprises a cholesterol or a cholesterol analogue. In some embodiments, the liposome comprises distearoyl phosphatidylcholine.

In some embodiments, the radiolabeled liposome comprises from about 0.01 mCi to about 400 mCi of the compound per 50 mg of lipid used to prepare the liposome.

In some embodiments, the liposome further comprises a chemotherapeutic agent, an antibiotic agent, or a treatment molecule, wherein the chemotherapeutic agent, the antibiotic agent, or the treatment molecule is incorporated or attached to the liposome.

In some embodiments, the disease or disorder is cancer. In some embodiments, the cancer is selected from lung cancer, breast cancer, colorectal cancer, prostate cancer, skin cancer, stomach cancer, bladder cancer, liver cancer, leukemia, lymphoma, ovarian cancer, pancreatic cancer, hepatocellular carcinoma, melanoma, sarcoma, head and neck cancer, glioma, glioblastoma, medulloblastoma, ependymoma, diffuse intrinsic pontine glioma, leptomeningeal metastases, and pediatric high-grade glioma. In some embodiments, the cancer is glioma. In some embodiments, the cancer is glioblastoma. In some embodiments, the cancer is recurrent glioblastoma. In some embodiments, the cancer is leptomeningeal metastases.

In some embodiments, the subject has not previously received treatment comprising bevacizumab.

In some embodiments, the radiolabeled liposome is administered via infusion of an infusate comprising the radiolabeled liposome.

In some embodiments, the radiolabeled liposome is administered via convection-enhanced delivery. In some embodiments, the convection-enhanced delivery comprises administration of the radiolabeled liposome via one or more catheters. In some embodiments, the convection-enhanced delivery comprises administration of the radiolabeled liposome via one catheter. In some embodiments, the convection-enhanced delivery comprises administration of the radiolabeled liposome via two catheters. In some embodiments, the convection-enhanced delivery comprises administration of the radiolabeled liposome via three catheters. In some embodiments, the convection-enhanced delivery comprises administration of the radiolabeled liposome via four catheters. In some embodiments, convection-enhanced delivery comprises

In some embodiments, the infusate is administered with a maximum flow rate of from about 1 μL min⁻¹ to about 50 μL min⁻¹. In some embodiments, the infusate is administered with a maximum flow rate of from about 5 μL min⁻¹ to about 20 μL min⁻¹. In some embodiments, the infusate is administered with a maximum flow rate of about 5 μL min⁻¹. In some embodiments, the infusate is administered with a maximum flow rate of about 10 μL min⁻¹. In some embodiments, the infusate is administered with a maximum flow rate of about 15 μL min⁻¹. In some embodiments, the infusate is administered with a maximum flow rate of about 20 μL min⁻¹. In some embodiments, the infusate is administered with a maximum flow rate of about 25 μL min⁻¹. In some embodiments, the infusate is administered with a maximum flow rate of about 30 μL min⁻¹. In some embodiments, the infusate is administered with a maximum flow rate of about 35 μL min⁻¹. In some embodiments, the infusate is administered with a maximum flow rate of about 40 μL min⁻¹. In some embodiments, the infusate is administered with a maximum flow rate of about 45 μL min⁻¹.

In some embodiments, the amount of radioactivity delivered by the radiolabeled liposome is from about 0.1 mCi to about 50 mCi. In some embodiments, the amount of radioactivity delivered by the radiolabeled liposome is from about 1 mCi to about 20 mCi. In some embodiments, the amount of radioactivity delivered by the radiolabeled liposome is about 1 mCi. In some embodiments, the amount of radioactivity delivered by the radiolabeled liposome is about 2 mCi. In some embodiments, the amount of radioactivity delivered by the radiolabeled liposome is about 4 mCi. In some embodiments, the amount of radioactivity delivered by the radiolabeled liposome is about 8 mCi. In some embodiments, the amount of radioactivity delivered by the radiolabeled liposome is about 13.4 mCi. In some embodiments, the amount of radioactivity delivered by the radiolabeled liposome is about 22.3 mCi. In some embodiments, the amount of radioactivity delivered by the radiolabeled liposome is about 31.2 mCi.

In some embodiments, the volume of infusate is from about 0.1 mL to about 25 mL. In some embodiments, the volume of infusate is from about 0.5 mL to about 10 mL. In some embodiments, the volume of infusate is from about 1 mL to about 5 mL. In some embodiments, the volume of infusate is from about 2 mL to about 15 mL. In some embodiments, the volume of infusate is from about 5 mL to about 10 mL. In some embodiments, the volume of infusate is from about 10 mL to about 15 mL. In some embodiments, the volume of infusate is from about 15 mL to about 20 mL. In some embodiments, the volume of infusate is about 0.66 mL. In some embodiments, the volume of infusate is about 1 mL. In some embodiments, the volume of infusate is about 1.32 mL. In some embodiments, the volume of infusate is about 2 mL. In some embodiments, the volume of infusate is about 2.64 mL. In some embodiments, the volume of infusate is about 3 mL. In some embodiments, the volume of infusate is about 4 mL. In some embodiments, the volume of infusate is about 5 mL. In some embodiments, the volume of infusate is about 5.28 mL. In some embodiments, the volume of infusate is about 6 mL. In some embodiments, the volume of infusate is about 7 mL. In some embodiments, the volume of infusate is about 8 mL. In some embodiments, the volume of infusate is about 8.8 mL. In some embodiments, the volume of infusate is about 9 mL. In some embodiments, the volume of infusate is about 10 mL. In some embodiments, the volume of infusate is about 11 mL. In some embodiments, the volume of infusate is about 12 mL. In some embodiments, the volume of infusate is about 12.3 mL. In some embodiments, the volume of infusate is about 13 mL. In some embodiments, the volume of infusate is about 14 mL. In some embodiments, the volume of infusate is about 15 mL. In some embodiments, the volume of infusate is about 16 mL. In some embodiments, the volume of infusate is about 16.35 mL. In some embodiments, the volume of infusate is about 17 mL. In some embodiments, the volume of infusate is about 18 mL. In some embodiments, the volume of infusate is about 18.5 mL. In some embodiments, the volume of infusate is more than about 18.5 mL. In some embodiments, the volume of infusate is delivered to a single hemisphere of the brain (e.g., comprising glioblastoma). In some embodiments, the volume of infusate is delivered to both hemispheres of the brain (e.g., comprising glioblastoma).

In some embodiments, the amount of radioactivity delivered by the radiolabeled liposome per volume of infusate is from about 0.1 mCi mL⁻¹ to about 50 mCi mL⁻¹. In some embodiments, the amount of radioactivity delivered by the radiolabeled liposome per volume of infusate is from about 0.5 mCi mL⁻¹ to about 10 mCi mL⁻¹. In some embodiments, the amount of radioactivity delivered by the radiolabeled liposome per volume of infusate is from about 1 mCi mL⁻¹ to about 5 mCi mL⁻¹. In some embodiments, the amount of radioactivity delivered by the radiolabeled liposome per volume of infusate is from about 1 mCi mL⁻¹ to about 10 mCi mL⁻¹. In some embodiments, the amount of radioactivity delivered by the radiolabeled liposome per volume of infusate is from about 2 mCi mL⁻¹ to about 10 mCi mL⁻¹. In some embodiments, the amount of radioactivity delivered by the radiolabeled liposome per volume of infusate is from about 4 mCi mL⁻¹ to about 10 mCi mL⁻¹. In some embodiments, the amount of radioactivity delivered by the radiolabeled liposome per volume of infusate is from about 5 mCi mL⁻¹ to about 10 mCi mL⁻¹. In some embodiments, the amount of radioactivity delivered by the radiolabeled liposome per volume of infusate is from about 1 mCi mL⁻¹ to about 3 mCi mL⁻¹. In some embodiments, the amount of radioactivity delivered by the radiolabeled liposome per volume of infusate is about 1 mCi mL⁻¹. some embodiments, the amount of radioactivity delivered by the radiolabeled liposome per volume of infusate is about 1.5 mCi mL⁻¹. In some embodiments, the amount of radioactivity delivered by the radiolabeled liposome per volume of infusate is about 2 mCi mL⁻¹. In some embodiments, the amount of radioactivity delivered by the radiolabeled liposome per volume of infusate is about 2.5 mCi mL⁻¹. In some embodiments, the amount of radioactivity delivered by the radiolabeled liposome per volume of infusate is about 3 mCi mL⁻¹. In some embodiments, the amount of radioactivity delivered by the radiolabeled liposome per volume of infusate is about 4 mCi mL⁻¹. In some embodiments, the amount of radioactivity delivered by the radiolabeled liposome per volume of infusate is about 5 mCi mL⁻¹. In some embodiments, the amount of radioactivity delivered by the radiolabeled liposome per volume of infusate is about 6 mCi mL⁻¹. In some embodiments, the amount of radioactivity delivered by the radiolabeled liposome per volume of infusate is about 7 mCi mL⁻¹. In some embodiments, the amount of radioactivity delivered by the radiolabeled liposome per volume of infusate is about 8 mCi mL⁻¹. In some embodiments, the amount of radioactivity delivered by the radiolabeled liposome per volume of infusate is about 9 mCi mL⁻¹. In some embodiments, the amount of radioactivity delivered by the radiolabeled liposome per volume of infusate is about 10 mCi mL⁻¹.

In some embodiments, the method further comprises imaging the radiolabeled liposome concomitant with administration. In some embodiments, the method further comprises imaging the radiolabeled liposome subsequent to administration.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the absorbed dose of ¹⁸⁶Re nanoliposomes to the tumor volume for patients previously treated with bevacizumab and bevacizumab-naïve patients.

FIG. 2 shows the tumor volume for each patient treated with ¹⁸⁶Re nanoliposomes.

FIG. 3 shows the ratio of treated volume to volume of infusate versus the volume of infusate following treatment with ¹⁸⁶Re nanoliposomes.

FIG. 4 shows the difference in survival following treatment with ¹⁸⁶Re nanoliposomes between patients previously treated with bevacizumab and bevacizumab-naïve patients.

FIG. 5 shows the mean absorbed dose to brain, total body, and tumor volume for the various dose levels of ¹⁸⁶Re nanoliposomes.

FIG. 6 shows baseline magnetic resonance images (MRIs) and single-photon emission computerized tomography (SPECT) images following treatment with ¹⁸⁶Re nanoliposomes.

FIG. 7 shows magnetic resonance images (MRIs) and perfusion scan images at baseline and 56 days following treatment with ¹⁸⁶Re nanoliposomes.

FIG. 8 shows a 3D image demonstrating the extent of radiation delivered (measured in absorbed dose) through 8 days posttreatment with ¹⁸⁶Re nanoliposomes.

FIG. 9 shows a chart demonstrating the overall survival of subjects comprising varying average absorbed dose and percent TuV/TrV.

FIG. 10 shows an image comprising baseline MRIs, and SPECT images at the end of infusion, 24 hours following infusion, 120 hours following infusion, and 192 hours following infusion.

FIG. 11 shows an image comprising baseline MRIs, and SPECT images after 20% infusion, at the end of infusion, 24 hours following infusion, 120 hours following infusion, and 192 hours following infusion.

FIG. 12 shows a Kaplan Meier curve comparing the overall survival of patients receiving greater than 100 Gy as compared to patients receiving less than 100 Gy.

FIG. 13 shows a baseline MRI and multiple SPECT images taken at various time points.

FIG. 14A shows a 3D dose distribution of ¹⁸⁶Re nanoliposomes (e.g., ¹⁸⁶RNL) brachytherapy.

FIG. 14B shows a dose volume histogram comprising the percent coverage on the Y-axis and the absorbed dose (in Gy) on the X-axis for multiple patients.

FIG. 14C shows an isodose distribution of a stereotactic body radiation therapy (SBRT) dose as a comparator.

FIG. 15 shows baseline magnetic resonance images (MRIs) and single-photon emission computerized tomography (SPECT) images following treatment with 186Re nanoliposomes.

FIG. 16 shows MRI scans of tumor response observed to Day 362.

FIG. 17 shows a process of convection enhanced delivery to treat GBM.

FIG. 18 shows efficacy and survival data for ReSPECT-GBM.

FIG. 19 shows SPECT CT, planar imaging and CSF Liquid Biopsy results for a patient post 186RNL treatment.

FIGS. 20A and 20B shows a process of producing a Rhenium 188 NanoLiposome and a Rhenium 188 NanoLiposome Biodegradable Alginate Microsphere.

FIG. 21 shows an approach for a non-surgical locoregional treatment option for solid organ tumors.

FIG. 22 shows a plot of radiation density and a table of density versus volume of infusion

FIG. 23 shows a table of patient versus volume of infusion.

FIG. 24 shows a RNL Kaplan-Meier plot comparing the overall survival of patients receiving greater than 100 Gy as compared to patients receiving less than 100 Gy up to 40 months.

FIG. 25 shows a RNL Kaplan-Meier plot comparing the overall survival of GBM patients receiving greater than 100 Gy as compared to patients receiving less than 100 Gy up to 42 months.

FIG. 26 shows a mortality plot with dose and tumor coverage.

FIG. 27 shows a radiotherapeutic formulation of rhenium (186Re) obisbemeda and a rhenium-188 nanoliposome biodegradable alginate microsphere.

FIG. 28 shows a plot of tumor retention based on percent injected activity over time for 186Re-NanoLiposomes, 186Re-BMEDA, and 186Re-Perrenate (CED Intratumoral Administration in Head/Neck SCC Xenografts in Nude Rats).

FIG. 29 shows a plot of tumor response based on tumor volume over time for 186Re-NanoLiposomes, 186Re-BMEDA, 186Re-Perrenate, and a NanoLiposome control (CED Intratumoral Administration in Head/Neck SCC Xenografts in Nude Rats).

FIG. 30 shows proportion alive (overall survival) by the percent volume of the tumor treated versus months.

FIG. 31 shows proportion alive (overall survival) by the average absorbed dose by the tumor versus months.

FIG. 32 shows leptomeningeal administration of rhenium (186Re) obisbemeda targeted to the CSF and rapid diffusion.

FIG. 33 shows updates on the ReSPECT-LM Clinical Trial.

### DETAILED DESCRIPTION OF THE INVENTION

### Definitions

Unless defined otherwise, all technical and scientific terms used herein have the same meaning as is commonly understood by one of skill in the art to which this disclosure belongs.

As used herein, the singular form “a”, “an” and “the” includes plural references unless the context clearly dictates otherwise.

As used herein, the term “radionuclide” refers to any element that emits radiation. Examples of radiation that can be emitted from a radionuclide include, but are not limited to, α-emission, β-emission, γ-emission, x-ray-emission, conversion electron emission, or Auger electron emission. The radiation that is emitted from the radionuclide can be detected and measured using techniques known in the art (see Goins and Phillips “The use of scintigraphic imaging as a tool in the development of liposome formulations,” Progress in Lipid Research, 40, pp. 95-123, 2001, which is incorporated herein by reference in its entirety). Examples of radionuclides useful in the embodiments provided herein are disclosed in “Srivastava et al. in “Recent Advances in Radionuclide Therapy,” Seminars in Nuclear Medicine, Vol. XXXI, No. 4, pp. 330-341, (October), 2001, which is incorporated by reference in its entirety.

As used herein, a “radiolabeled liposome” refers to a liposome comprising a radiolabeled compound provided herein incorporated or attached to the liposome. The term “liposome” refers to any vesicle comprising a double membrane. “Liposome” includes unilamellar and multilamellar liposomes.

As used herein, the term “incorporated” refers to embedding a compound of Formula I in the double membrane of the liposome. Because the double membrane of liposomes is lipophilic, compounds with high lipophilicity can be trapped within the double membrane of the liposome.

A “pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative, such as those known in the art, for example, described in Remington's Pharmaceutical Sciences 16th edition, Osol, A. Ed. (1980).

As used herein, “treatment” or “treating” is an approach for obtaining beneficial or desired results including and preferably clinical results. For example, beneficial or desired clinical results include, but are not limited to, one or more of the following: decreasing symptoms resulting from the disease, increasing the quality of life of those suffering from the disease, decreasing the dose of other medications required to treat the disease, delaying the progression of the disease, and/or prolonging survival of individuals.

As used herein, an “effective dosage” or “effective amount” of drug, compound, or pharmaceutical composition is an amount sufficient to effect beneficial or desired results. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent and preferably stop) cancer cell infiltration into peripheral organs; inhibit (i.e., slow to some extent and preferably stop) tumor metastasis; inhibiting, to some extent, tumor growth; and/or relieving to some extent one or more of the symptoms associated with the disorder. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration. The term is intended to encompass radiolabeling.

As used herein, the term “administering” means oral administration, administration as a suppository, topical contact, intravenous, parenteral, intraperitoneal, intramuscular, intralesional, intrathecal, intranasal, convection (e.g., via convection-enhanced delivery) or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal compositions, intravenous infusion, transdermal patches, etc.

As used herein, the term “delivered”, and variations of thereof (e.g., “delivering”) are, used interchangeably with the term “administered”, and variations thereof (e.g., “administering”).

By “co-administer” it is meant that a compound described herein is administered at the same time, just prior to, or just after the administration of one or more additional therapies, for example, an anticancer agent as described herein. The compounds described herein can be administered alone or can be co-administered to the patient. Co-administration is meant to include simultaneous or sequential administration of the compound individually or in combination (more than one compound or agent). Thus, the preparations can also be combined, when desired, with other active substances (e.g. anticancer agents).

Co-administration includes administering one active agent (e.g. radiolabeled nanoliposomes described herein) within 0.5, 1, 2, 4, 6, 8, 10, 12, 16, 20, or 24 hours of a second active agent (e.g. anti-cancer agents). Also contemplated herein, are embodiments, where co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. Co-administration can be accomplished by co-formulation, i.e., preparing a single pharmaceutical composition including both active agents. In other embodiments, the active agents can be formulated separately. In some embodiments, the active and/or adjunctive agents are linked or conjugated to one another. In some embodiments, the compounds described herein are combined with treatments for cancer such as chemotherapy or radiation therapy.

“Patient,” “subject,” “patient in need thereof,” and “subject in need thereof” are herein used interchangeably and refer to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human. A “cancer-patient” is a patient suffering from, or prone to developing cancer.

Unless clearly indicated otherwise, the term “individual” as used herein refers to a mammal, including but not limited to, bovine, horse, feline, rabbit, canine, rodent, or primate (e.g., human). In some embodiments, an individual is a human. In some embodiments, an individual is a non-human primate such as chimpanzees and other apes and monkey species. In some embodiments, an individual is a farm animal such as cattle, horses, sheep, goats and swine; pets such as rabbits, dogs and cats; laboratory animals including rodents, such as rats, mice, and guinea pigs; and the like. In some embodiments, the invention find use in both human medicine and in the veterinary context.

“Disease” or “condition” refer to a state of being or health status of a patient or subject capable of being treated with the compounds or methods provided herein. In some embodiments, the disease as used herein refers to cancer.

“Chemotherapeutic” or “chemotherapeutic agent” is used in accordance with its plain ordinary meaning and refers to a chemical composition or compound having antineoplastic properties or the ability to inhibit the growth or proliferation of cells.

“Cancer model organism”, as used herein, is an organism exhibiting a phenotype indicative of cancer, or the activity of cancer causing elements, within the organism. The term cancer is defined above. A wide variety of organisms may serve as cancer model organisms, and include for example, cancer cells and mammalian organisms such as rodents (e.g. mouse or rat) and primates (such as humans). Cancer cell lines are widely understood by those skilled in the art as cells exhibiting phenotypes or genotypes similar to in vivo cancers. Cancer cell lines as used herein includes cell lines from animals (e.g. mice) and from humans.

### Radiolabeled Nanoliposomes

Brachytherapy can be useful in treating cancer selected from lung cancer, breast cancer, colorectal cancer, prostate cancer, skin cancer, stomach cancer, bladder cancer, liver cancer, leukemia, lymphoma, ovarian cancer, pancreatic cancer, hepatocellular carcinoma, melanoma, sarcoma, head and neck cancer, glioma, glioblastoma, medulloblastoma, ependymoma, diffuse intrinsic pontine glioma, thalamic tumors, brain stem tumors, leptomeningeal metastases, and pediatric high-grade glioma. In some embodiments, the cancer is a thalamic tumor. In some embodiments, the cancer is a brain stem tumor. In some embodiments, the cancer is glioma. In some embodiments, the cancer is glioblastoma. In some embodiments, the cancer is recurrent glioblastoma. In some embodiments, the cancer is leptomeningeal metastases.

Liposomes comprising phospholipids and/or sphingolipids may be used to deliver hydrophilic (water-soluble) or precipitated therapeutic compounds encapsulated within the inner liposomal volume and/or to deliver hydrophobic therapeutic agents dispersed within the hydrophobic bilayer membrane. In certain aspects the liposome comprises lipids selected from sphingolipids, ether lipids, sterols, phospholipids, phosphoglycerides, and glycolipids. In certain aspects, the lipid includes, for example, DSPC (1,2-distearoyl-sn-glycero-3-phosphocholine).

Liposomes are of considerable interest because of their value as carriers for diagnostic agents, particularly radiopharmaceuticals for tracer and imaging studies. There are many advantages of using liposomes as carriers of therapeutic radionuclides. Some advantages include (1) the biocompatibility of liposomes; (2) liposome particles of varying sizes with a uniform population size range can readily be achieved by using extrusion techniques; (3) the surface of liposomes can be modified with different kinds of functional groups; (4) the distribution of liposomes can be functional and microtargeted; and (5) the mechanism of radioisotope diffusion from liposomes can be monitored, which is helpful in delivering a uniform dose distribution in tumor tissues.

Radionuclides have been widely used as a non-invasive method for studying the distribution of drugs in vivo. However, attempts at labeling liposomes with radionuclides as imaging agents have produced variable results. Many radionuclides weakly bind to liposomes, causing radionuclide leaching from the liposome and resulting in inaccurate biodistribution data. Furthermore, the entrapment of water-soluble radionuclides within the liposome during manufacturing is relatively inefficient.

In an embodiment, occult tumor cells are responsible for tumor recurrences. In an embodiment, the targeted killing of both tumor and a specific rim of abnormal tissue in performed while sparing normal neuronal tissues and cells.

An embodiment of the disclosure is a method of delivering substantial increments of the composition in a patient receiving a prior maximum dose of radiation therapy for treatment of the patient's primary disease, secondary disease, or both. An embodiment of the disclosure is a method of delivering substantial increments of the composition in a patient receiving a prior maximum dose of radiation therapy for treatment of the patient's primary disease, secondary disease, or both. In an embodiment, the radiation is selected from beta radiation or alpha particle therapy comprising isotopes Ac225, Pb212 or Cu67. In an embodiment, the radiolabeled liposomal formulation delivers amounts of radiation plus dwell time sufficient to eradicate cancer cells in the cerebrospinal fluid, in the leptomeningeal lining, or both but does not damage surrounding brain or other systemic organs and tissues. In an embodiment, the composition comprising simultaneously delivering and visualizing radiotherapeutic delivery for the purpose of ensuring successful delivery or to making changes to key delivery parameters administration. In some embodiments, the subject receiving increments of radiation previously received a maximum dose of radiation. In some embodiments, the subject receiving increments of radiation previously received a maximum dose of radiation for the treatment of their primary and/or secondary disease(s). In some embodiments, the subject previously received a maximum dose of radiation for treatment of a cancer (e.g., glioblastoma). In some cases, the maximum dose of radiation comprises a maximum permissible dose (e.g., an upper limit of allowed radiation that a subject may receive without the risk of significant side effects).

In an embodiment, radiolabeled nanoliposomes (RNL) can be used to treat cancer. In an embodiment, RNL can be used to treat cancer via brachytherapy. In an embodiment, RNL can be used to treat glioblastomas, leptomeningeal metastases, thalamic tumors, and brain stem tumors.

In some embodiments, provided herein, are methods of delivering and visualizing (e.g., imaging) radiotherapeutic delivery. In some embodiments, the radiotherapeutic comprises the pharmaceutical composition disclosed herein. In some embodiments, provided herein, are methods of delivering and visualizing (e.g., imaging) the pharmaceutical composition (e.g., the radiolabeled liposome (e.g., 186Re nanoliposome))) delivery. In some embodiments, the method comprises ensuring delivery to the target. In some embodiments, the target is a cancer. In some embodiments, the target is a tumor. In some embodiments, the tumor is a primary tumor. In some embodiments, the tumor is a secondary tumor. In some embodiments, the method comprises modifying (e.g., making changes to) one or more delivery parameters during delivery. In some embodiments, delivering the radiotherapeutic (e.g., the pharmaceutical composition). In some embodiments, the method comprises modifying one or more delivery parameters during delivery and ensuring delivery of the pharmaceutical composition to the target during delivery.

In some embodiments, visualizing (e.g., imaging) comprises any of the imaging methods disclosed herein (e.g., SPECT, SPECT-CT, MRI, etc.). In some embodiments, visualizing (e.g., the radiotherapeutic (radiolabeled liposome (e.g., mRe)) delivery) comprises imaging the whole body. In some embodiments, visualizing comprises imaging of a region (e.g., locoregion) of the body (e.g., the head). In some embodiments, visualizing comprises imaging the target (e.g., tumor). In some embodiments, visualizing comprises imaging of a primary tumor. In some embodiments, visualizing comprises imaging of a secondary tumor. In some embodiments, visualizing comprises imaging of a primary tumor and a secondary tumor. In some embodiments, visualizing comprises imaging of a tumor and the area surrounding the tumor (e.g., the region in which the tumor is located and/or within 6 inches of the tumor). In some embodiments, visualizing comprises imaging one or more organs (e.g., comprising a tumor and/or within the region of the tumor). In some embodiments, visualizing comprises imaging of a primary tumor or a disease area. In some embodiments, surrogate markers are measured in conjunction with visualization of the disease area.

### Rhenium-186 Isotope

Dual energy emitter: beta (cytotoxic) & gamma (imaging)
Short average path length (1.8 mm): high precision
Low dose rate: safer for normal tissues
High radiation density: overwhelms innate DNA repair mechanisms

### Rhenium-188

Dual energy emitter: beta (cytotoxic) & gamma (imaging)
Short average path length (3.1 mm): offers greater precision
Low dose rate: safer for normal tissues
High radiation density: overwhelms innate DNA repair mechanisms
Generator-produced for quick availability

The therapeutic agent can be a chemotherapeutic agent or a radiotherapeutic agent. In certain aspects the chemotherapeutic agent is a taxane, epothilones, or vinca alkaloid. In certain aspects the radiotherapeutic agent is ¹³¹I, ⁹⁰Y, ⁹⁹mTc, ¹⁷Lu, ¹⁸⁶Re, ¹⁸⁸Re, ¹²⁵I, ¹²³I, or any combination thereof. In other aspects the radiotherapeutic agent can be one or more of Bismuth-213, Cesium-131, Chromium-51, Cobalt-60, Dysprosium-165, Erbium-169, Holmium-166, Iodine-125, Iodine-131, Iridium-192, Iron-59, Lead-212, Lutetium-177, Molybdenum-99, Palladium-103, Phosphorus-32, Potassium-42, Radium-223, Rhenium-186, Rhenium-188, Samarium-153, Scandium-47, Selenium-75, Sodium-24, Strontium-89, Technetium-99m, Thorium-227, Xenon-133, Ytterbium-169, Ytterbium-177, Yttrium-90, Actinium-225, Astatine-211, Bismuth-212, Carbon-11, Fluorine-18, Nitrogen-13, Oxygen-15, Cobalt-57, Copper-64, Copper-67, Gallium-67, Gallium-68, Germanium-68, Indium-111, Iodine-123, Iodine-124, Krypton-81m, Rubidium-82, Strontium-82, and/or Thallium-201.

In an embodiment, the liposomes encapsulate a therapeutic agent complexed with a loading agent, diagnostic agent complexed with a loading agent, or a combination thereof, wherein the loading efficiency of a therapeutic agent is 10, 20, 30, 40, 50, 60, 70, 80, 90, to 100%, including all ranges and values there between. In certain aspects the therapeutic agent or diagnostic agent is one or more of Bismuth-213, Cesium-131, Chromium-51, Cobalt-60, Dysprosium-165, Erbium-169, Holmium-166, Iodine-125, Iodine-131, Iridium-192, Iron-59, Lead-212, Lutetium-177, Molybdenum-99, Palladium-103, Phosphorus-32, Potassium-42, Radium-223, Rhenium-186, Rhenium-188, Samarium-153, Scandium-47, Selenium-75, Sodium-24, Strontium-89, Technetium-99m, Thorium-227, Xenon-133, Ytterbium-169, Ytterbium-177, Yttrium-90, Actinium-225, Astatine-211, Bismuth-212, Carbon-11, Fluorine-18, Nitrogen-13, Oxygen-15, Cobalt-57, Copper-64, Copper-67, Gallium-67, Gallium-68, Germanium-68, Indium-111, Iodine-123, Iodine-124, Krypton-81m, Rubidium-82, Strontium-82, and/or Thallium-201.

Substances of note that can be encapsulated in include radiotherapeutics (e.g., rhenium-188), radiolabels (e.g., technetium-99m), chemotherapeutics (doxorubicin), magnetic particles (e.g., 10 μm iron nanoparticles), and radio-opaque material (e.g., iodine contrast). In certain aspects, rhenium-188 liposomes can be used for treatment of liver tumors, specifically hepatocellular carcinoma (HCC). In a more particular aspect HCC treatment can be through radioembolization, where the microspheres block the blood supply to the tumor from the artery, while the rhenium-188 also delivers a high dose of radiation that is primarily targeted to the cancer cells.

In an embodiment, the radiotherapeutic agent is 209Bi, 211Bi, 212Bi, 213Bi, 210Po, 211Po, 212Po, 214Po, 215Po, 216Po, 218Po, 215At, 217At, 218At, 218Rn, 219Rn, 220Rn, 222Rn, 226Rn, 221Fr, 223Ra, 224Ra, 226Ra, 225Ac, 227Ac, 227Th, 228Th, 229Th, 230Th, 232Th, 231 Pa, 233U, 234U, 235U, 236U, 238U, 237Np, 238Pu, 239Pu, 240Pu, 244Pu, 241Am

244Cm, 245Cm, 248Cm, 249Cf, 252Cf or alpha decay isotopes.

In certain aspects, a radiotherapeutic agent includes a radiolabel or radiotherapeutic such as a beta emitter (¹³¹I, ⁹⁰Y, ¹⁷⁷Lu, ¹⁸⁶Re, ¹⁸⁸Re, any one of which can be specifically excluded) or gamma emitter (¹²⁵I, ¹²³I, ^{99m}Tc), or any combination thereof. In certain aspects, the radiotherapeutic agent is ¹⁸⁸Re. Furthermore, the term “radiotherapeutic” may be taken to more broadly encompass any radioactively-labeled moiety, and may include any liposome associated with or comprising a radionuclide. Nuclear reactors are the source of many radioisotopes while are sourced from cyclotrons. In general, nuclear fission [reactors] produce neutron rich isotopes while neutron depleted isotopes, for example PET radionuclides are cyclotron produced [cyclotron energy ˜10-20 MeV for usual PET positron isotopes whereas single photon products usually require higher cyclotron energy [˜30 MeV]. In certain embodiments the radiotherapeutic can be a reactor radioisotope or a cyclotron radioisotope. Reactor radioisotopes can include (1) a therapeutic [Rx], both beta and alpha and low energy x-rays [for brachytherapy] and/or (2) a diagnostic [Dx], both positron and single photon. The Rx or Dx listed here are exemplary embodiments of how the radioisotopes can be used. The scope of the invention includes utilizing the radioisotopes listed here in other Rx or Dx. Reactor radioisotopes include, but are not limited to: Bismuth-213 (alpha), Cesium-131 (x-rays brachyRx), Chromium-51 (Dx), Cobalt-60 (historically EBRT now universally used for sterilizing; historically HSACo-60 for brain cancer Rx), Dysprosium-165 (beta Rx), Erbium-169 (beta Rx), Holmium-166 (beta Rx), Iodine-125 (low energy x-rays Rx brachytherapy and RIA applications), Iodine-131 (Beta Rx [fission product]; has an imaging gamma, albeit high energy), Iridium-192 (beta Rx; often in wire form for brachytherapy, e.g., prostate), Iron-59 (Dx historically iron metabolism studies), Lead-212 (alpha Rx), Lutetium-177 (Rx beta; has gamma emission for imaging), Molybdenum-99 (Dx—parent of Tc99m [fission product]), Palladium-103 (Rx low energy x-rays example of permanent implant brachytherapy), Phosphorus-32 (beta Rx; historic Rx of polycythemia vera), Potassium-42 (Dx historic measure of exchangeable K+ for coronary blood flow), Radium-223 (Rx alpha; historic brachyRx with low-energy x-rays), Rhenium-186 (beta Rx with imaging photon; historic Rx bone pain), Rhenium-188 (beta Rx; historic coronary arteries via stent), Samarium-153 (beta Rx; historic product [Quadramet] for bone pain/metastisis), Scandium-47 (beta Rx with imaging capability; —Lu-177; produced by irradiating Ca-46 to produce Ca-47 which decays to Sc-47), Selenium-75 (Dx; historic seleno-methionine for GI study), Sodium-24 (Dx historic electrolytes study), Strontium-89 (Rx bone pain and metastisis [fission product]), Technetium-99m (Dx; workhorse Dx isotope in nuclear medicine; produced in generator from Mo-99), Thorium-227 (Rx alpha; decays to Ra-223 another alpha Rx), Xenon-133 (Dx [a gas-fission product]), Ytterbium-169 (Dx; used before In-111 for CSF flow studies), Ytterbium-177 (Rx precursor of Lu-177 via Yb-176 neutron irradiation), and Yttrium-90 (Rx pure beta emitter [fission product]). Cyclotron radioisotopes include, but are not limited to: Actinium-225 (Rx alpha), Astatine-211 (Rx alpha), Bismuth-212 (Rx alpha), Carbon-11 (Dx positron/PET), Fluorine-18 (Dx positron/PET), Nitrogen-13 (Dx positron/PET), Oxygen-15 (Dx positron/PET), Cobalt-57 (Dx in-vitro Dx kits), Copper-64 (Dx positron; historic studies copper metabolism), Copper-67 (Rx beta), Gallium-67 (Dx single photon), Gallium-68 (Dx positron), Germanium-68 (Dx—parent for Ga-68 generator), Indium-111 (Dx), Iodine-123 (Dx, no beta emission), Iodine-124 (Dx positron), Krypton-81m (Dx [gas generator produced from Rb-81 at bedside T1/2=13 seconds]), Rubidium-82 (Dx positron potassium analog for perfusion imaging; generator produced at patient T1/2=75 seconds), Strontium-82 (Dx—parent for the Rb-82 generator), and Thallium-201 (Dx). The liposome can be associated with aradionuclide through a chelator, direct chemical bonding, or some other means such as a linker protein.

In an embodiment, a chelating agent can form a chelating complex with the transition metal or the radiolabeled agent, such as the radionuclide.

In an embodiment, chelators may be selected from the group comprising 1,4,7,10-tetraazacyclododecane-1,4,7,10-tetraacetic acid (DOTA) and derivatives thereof; 1,4,8,11-tetraazacyclotetradecane (cyclam) and derivatives thereof; 1,4,7,10-tetraazacyclododecane (cyclen) and derivatives thereof; 1,4-ethano-1,4,8,11-tetraazacyclotetradecane (et-cyclam) and derivatives thereof; 1,4,7,11-tetraazacyclotetradecane (isocyclam) and derivatives thereof; 1,4,7,10-tetraazacyclotridecane ([13]aneN4) and derivatives thereof; 1,4,7,10-tetraazacyclododecane-1,7-diacetic acid (DO2A) and derivatives thereof; 1,4,7,10-tetraazacyclododecane-1,4,7-triacetic acid (DO3A) and derivatives thereof; 1,4,7,10-tetraazacyclododecane-1,7-di(methanephosphonic acid) (DO2P) and derivatives thereof; 1,4,7,10-tetraazacyclododecane-1,4,7-tri(methanephosphonic acid) (DO3P) and derivatives thereof; 1,4,7,10-tetraazacyclododecane-1,4,7,10-tetra(methanephosphonic acid) (DOTP) and derivatives thereof; ethylenediaminetetraacetic acid (EDTA) and derivatives thereof: diethylenetriaminepentaacetic acid (DTPA) and derivatives thereof; 1,4,8,11-tetraazacyclotetradecane-1,4,8,11-tetraacetic acid (TETA) and derivatives thereof, or other adamanzanes and derivates thereof.

In an embodiment, the chelator selected from the group consisting of macrocyclic compounds comprising adamanzanes; 1,4,7,10-tetraazacyclododecane ([12]aneN4) or a derivative thereof; 1,4,7,10-tetraazacyclotridecane ([13]aneN4) or a derivative thereof; 1,4,8,11-tetraazacyclotetradecane ([14]aneN4) or a derivative thereof; 1,4,8,12-tetraazacyclopentadecane ([15]aneN4) or a derivative thereof; 1,5,9,13-tetraazacyclohexadecane ([16]aneN4) or a derivative thereof; and other chelators capable of binding metal ions such as ethylene-diamine-tetraacetic-acid (EDTA) or a derivative thereof, diethylene-triamine-penta-acetic acid (DTPA) or a derivative thereof.

In an embodiment, the chelator selected from the group consisting of 1,4-ethano-1,4,8,11-tetraazacyclotetradecane (et-cyclam) or a derivative thereof; 1,4,7,11-tetraazacyclotetradecane (iso-cyclam) or a derivatives thereof; 1,4,7,10-tetraazacyclododecane-1,4,7,10-tetraacetic acid (DOTA) or a derivative thereof; 2-(1,4,7,10-tetraazacyclododecan-1-yl)acetate (DO1A) or a derivative thereof; 2,2′-(1,4,7,10-tetraazacyclododecane-1,7-diyl) diacetic acid (DO2A) or a derivative thereof; 2,2′,2″-(1,4,7,10-tetraazacyclododecane-1,4,7-triyl)triacetic acid (DO3A) or a derivative thereof; 1,4,7,10-tetraazacyclododecane-1,4,7,10-tetra(methanephosphonic acid) (DOTP) or a derivative thereof; 1,4,7,10-tetraazacyclododecane-1,7-di(methanephosphonic acid) (DO2P) or a derivative thereof; 1,4,7,10-tetraazacyclododecane-1,4,7-tri(methanephosphonic acid) (DO3P) or a derivative thereof; 1,4,8,11-15 tetraazacyclotetradecane-1,4,8,11-tetraacetic acid (TETA) or a derivative thereof; 2-(1,4,8,11-tetraazacyclotetradecane-1-yl)acetic acid (TE1A) or a derivative thereof; 2,2′-(1,4,8,11-tetraazacyclotetradecane-1,8-diyl)diacetic acid (TE2A) or a derivative thereof; and other adamanzanes or derivates thereof.

Rhenium-186 (¹⁸⁶Re) is a β-ray-emitting therapeutic radionuclide with an approximately 90-hour half-life, 1.8-mm radiation path range, and high β/γ-energy ratio suitable for cancer brachytherapy. Additionally, ¹⁸⁶Re has an energy of gamma ray sufficient to allow imaging of in vivo drug behavior with standard SPECT/CT. Therapeutic radionuclides require a carrier to ensure they are sequestered within the tumor and slowly redistributed. Liposomal nanoparticles (nanoliposomes) provide a means of encapsulating radionuclides and assisting in sustained intratumoral accumulation. We have successfully developed a method of loading ¹⁸⁶Re into nanoliposomes with high efficiency and specific activity. The process results in a markedly higher level of specific activity than has been previously described and has the potential to provide a markedly higher delivered therapeutic radiation dose with decreased toxicity.

In certain aspects, a contrast or imaging agent includes, but is not limited to a transition metal, carbon nanomaterials such as carbon nanotubes, fullerene and graphene, near-infrared (NIR) dyes such as indocyanine green (ICG), and gold nanoparticles. Transition metal refers to a metal in Group 3 to 12 of the Periodic Table of Elements, such as titanium (Ti), vanadium (V), niobium (Nb), tantalum (Ta), chromium (Cr), molybdenum (Mo), tungsten (W), manganese (Mn), iron (Fe), ruthenium (Ru), osmium (Os), iridium (Ir), nickel (Ni), copper (Cu), technetium (Tc), rhenium (Re), cobalt (Co), rhodium (Rh), iridium (Ir), palladium (Pd), platinum (Pt), silver (Ag), gold (Au), a lanthanide such as europium (Eu), gadolinium (Gd), lanthanum (La), ytterbium (Yb), and erbium (Er), or a post-transition metal such as gallium (Ga), and indium (In). In one aspect, the imaging modality is selected from the group comprising, Positron Emission Tomography (PET), Single Photon Emission Tomography (SPECT), Computed Tomography (CT), Magnetic Resonance Imaging (MRI), Ultrasound Imaging (US), and Optical Imaging. In another aspect of the invention, the imaging modality is Positron Emission Tomography (PET). The imaging agent includes, but is not limited to a radiolabel, a fluorophore, a fluorochrome, an optical reporter, a magnetic reporter, an X-ray reporter, an ultrasound imaging reporter or a nanoparticle reporter. In another aspect of the invention, the imaging agent is a radiolabel selected from the group comprising a radioisotopic element selected from the group consisting: of astatine, bismuth, carbon, copper, fluorine, gallium, indium, iodine, lutetium, nitrogen, oxygen, phosphorous, rhenium, rubidium, samarium, technetium, thallium, yttrium, and zirconium. In another aspect, the radiolabel is selected from the group comprising zirconium-89 (⁸⁹Zr), iodine-124 (¹²⁴I), iodine-131 (¹³¹I), iodine-125 (¹²⁵I) iodine-123 (¹²³I), bismuth-212 (²¹²Bi), bismuth-213 (²¹³Bi), astatine-211 (²¹¹At), copper-67 (⁶⁷Cu), copper-64 (⁶⁴Cu), rhenium-186 (¹⁸⁶Re), rhenium-188 (¹⁸⁸Re), phosphorus-32 (³²P), samarium-153 (¹⁵3Sm), lutetium-177 (¹⁷⁷Lu), technetium-99m (^{99m}Tc), gallium-67 (⁶⁷Ga), indium-111 (¹¹¹In), thallium-201 (²⁰¹T1) carbon-11, nitrogen-13 (¹³N), oxygen-15 (¹⁵O), fluorine-18 (¹⁸F), and rubidium-82 (⁸²Ru).

Provided herein, in one aspect, is a radiolabeled liposome comprising a liposome and a compound of Formula I:

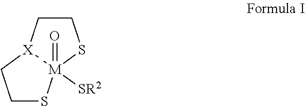

or a pharmaceutically acceptable salt thereof, wherein:
   M is ^{99m}Tc, ¹⁸⁶Re, ¹⁸⁸Re, or a combination thereof;
   X is NR¹;
   R¹ is CH₂CH₂NEt₂ or CH₂CH₂CH₂CH₃; and
   R² is CH₂CH₂N(CH₂CH₂SH)(CH₂CH₂NEt₂) or
CH₂CH₂N(CH₂CH₂SH)(CH₂CH₂CH₂CH₃).

In some embodiments, R¹ is CH₂CH₂NEt₂. In some embodiments, R¹ is CH₂CH₂CH₂CH₃. In some embodiments, R² is CH₂CH₂N(CH₂CH₂SH)(CH₂CH₂NEt₂). In some embodiments, R² is CH₂CH₂N(CH₂CH₂SH)(CH₂CH₂CH₂CH₃). In some embodiments, R¹ is CH₂CH₂NEt₂ and R² is CH₂CH₂N(CH₂CH₂SH)(CH₂CH₂NEt₂). In some embodiments, R¹ is CH₂CH₂CH₂CH₃ and R² is CH₂CH₂N(CH₂CH₂SH)(CH₂CH₂CH₂CH₃).

In some embodiments, M is ^{99m}Tc. In some embodiments, M is ¹⁸⁶Re. In some embodiments, M is ¹⁸⁸Re. In some embodiments, when M is ¹⁸⁶Re and the liposomes comprise nanoliposomes the radiolabeled liposome is referred to as ¹⁸⁶Re nanoliposomes.

In some embodiments, the compound is incorporated or attached to the liposome (e.g., nanoliposome).

In some embodiments, the liposome further comprises a drug that is incorporated within the liposome.

In some embodiments, the drug is a compound comprising at least one thiol group. In some embodiments, the drug reacts with the compound. In some embodiments, the drug comprises glutathione, cysteine, N-acetyl cysteine, 2-mercaptosuccinic acid, 2,3-dimercaptosuccinic acid, captopril, or a combination thereof. In some embodiments, the drug comprises glutathione. In some embodiments, the drug comprises cysteine. In some embodiments, the drug comprises N-acetyl cysteine. In some embodiments, the drug comprises 2-mercaptosuccinic acid. In some embodiments, the drug comprises 2,3-dimercaptosuccinic acid. In some embodiments, the drug comprises captopril. In some embodiments, the drug comprises a combination.

In some embodiments, the liposome comprises a lipid. In one embodiment, the liposome is a nanoliposome. In some embodiments, the liposome comprises a phospholipid.

In some embodiments, the liposome comprises a cholesterol or a cholesterol analogue. In some embodiments, the liposome comprises a cholesterol. In some embodiments, the liposome comprises a cholesterol analogue. In some embodiments, the liposome comprises distearoyl phosphatidylcholine.

In some embodiments, the liposome comprises a cholesterol or a cholesterol analogue. In some embodiments, the liposome comprises a cholesterol. In an embodiment, the liposome comprises a cholesterol. In an embodiment, the liposome comprises distearoyl phosphatidylcholine. In an embodiment, the composition delivers gamma and beta radiation. In an embodiment, the drug release beta-rays, gamma-rays, or a combination thereof.

### Pharmaceutical Compositions

A pharmaceutical composition of the present disclosure may be formulated in any suitable pharmaceutical formulation. A pharmaceutical composition of the present disclosure typically contains an active ingredient, and one or more pharmaceutically acceptable excipients or carriers, including but not limited to: inert solid diluents and fillers, diluents, sterile aqueous solution and various organic solvents, permeation enhancers, solubilizers, and adjuvants. A composition of the present disclosure may be formulated in any suitable pharmaceutical formulation. A pharmaceutical composition comprising ¹⁸⁶Re nanoliposomes may be referred to as ¹⁸⁶Rhenium-lipid nanoparticles (¹⁸⁶RNL). In some embodiments, the pharmaceutical composition comprises ¹⁸⁶Re nanoliposomes. In some embodiments, the pharmaceutical composition comprises ¹⁸⁶Re nanoliposomes, ¹⁸⁸Re nanoliposomes, ^{99m}Tc nanoliposomes, or any combination thereof. In some embodiments, the pharmaceutical composition results in sustained intratumoral accumulation.

Pharmaceutical compositions may be provided in any suitable form, which may depend on the route of administration. In some embodiments, the pharmaceutical composition disclosed herein can be formulated in dosage form for administration to a subject. In some embodiments, the pharmaceutical composition is formulated for oral, intravenous, intraarterial, aerosol, parenteral, buccal, topical, transdermal, rectal, intramuscular, subcutaneous, intraosseous, intranasal, intrapulmonary, transmucosal, inhalation, intraventricular, into the leptomeningeal space (e.g, for the treatment of leptomeningeal metastases), and/or intraperitoneal administration. In some embodiments, the pharmaceutical composition is formulated for administration via convection-enhanced delivery (CED). In some embodiments, the pharmaceutical composition is suitable for infusion via ventricular reservoir

The amount of a radiolabeled nanoliposomes (e.g., ¹⁸⁶Re nanoliposomes) administered will be dependent on the mammal being treated, the severity of the disorder or condition, the rate of administration, the disposition of the compound and the discretion of the prescribing physician.

In some embodiments, the radiolabeled nanoliposomes can be administered as part of a therapeutic regimen that comprises administering one or more second agents (e.g. 1, 2, 3, 4, 5, or more second agents), either simultaneously or sequentially with the radiolabeled nanoliposomes. When administered sequentially, the radiolabeled nanoliposomes may be administered before or after the one or more second agents. When administered simultaneously, the radiolabeled nanoliposomes and the one or more second agents may be administered by the same route (e.g. convection-enhanced delivery to the same location), by a different route (e.g. a tablet taken orally while receiving convection-enhanced delivery), or as part of the same combination (e.g. a solution comprising radiolabeled nanoliposomes and one or more second agents). In some embodiments, the radiolabeled nanoliposomes (e.g., ¹⁸⁶Re nanoliposomes) are delivered within a pharmaceutical composition. In some embodiments, the pharmaceutical composition comprises ¹⁸⁶Re nanoliposomes.

A combination treatment according to the invention may be effective over a wide dosage range. The exact dosage will depend upon the agent selected, the route of administration, the form in which the compound is administered, the subject to be treated, the body weight of the subject to be treated, and the preference and experience of the attending physician.

In some embodiments, the pharmaceutical composition comprises one or more surfactants. Surfactants which can be used to form pharmaceutical composition and dosage forms of the disclosure include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; camitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; camitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl camitines, palmitoyl camitines, myristoyl camitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group of vegetable oils, hydrogenated vegetable oils, and triglycerides.

In one embodiment, the composition may include a solubilizer to ensure good solubilization and/or dissolution of the radiolabeled nanoliposomes of the present disclosure and to minimize precipitation of the radiolabeled nanoliposomes of the present disclosure. This can be especially important for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. If present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

### Pharmaceutical Compositions for Infusion

In some embodiments, the disclosure provides a pharmaceutical composition for infusion, such as convection-enhanced delivery, comprising radiolabeled nanoliposomes (e.g., ¹⁸⁶Re nanoliposomes) and a pharmaceutical excipient suitable for injection. Components and amounts of agents in the composition are as described herein. In some embodiments, the pharmaceutical composition is suitable for infusion via a ventricular reservoir.

The forms in which the radiolabeled nanoliposomes of the present disclosure may be incorporated for administration by infusion include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for infusion. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile infusable solutions are prepared by incorporating the radiolabeled nanoliposomes of the present disclosure in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile infusable solutions, certain desirable methods of preparation are vacuum-drying and freeze-drying techniques which yield a powder of the active ingredient plus any additional desired ingredient from a previously sterile-filtered solution thereof.

In some embodiments, the pharmaceutical composition is administered for about 5 minutes, about 10 minutes, about 15 minutes, about 20 minutes, about 30 minutes, about 45 minutes, about 1 hour, about 1.5 hours, or about 2 hours.

### Convection-Enhanced Delivery

Convection-Enhanced Delivery (CED) involves stereotactic placement of one or more catheters through cranial burr holes directly into brain tissue. A therapeutic agent can be continuously administered through the catheters by a microinfusion delivery system to create a positive pressure gradient at the catheter tip. As the pressure is maintained, it creates fluid convection or flow to supplement diffusion through the extracellular spaces and enhance the distribution of the therapeutic agent to the targeted area. The goals of CED can be to provide homogenous distribution of a therapeutic agent to a larger volume of brain tissue, to provide higher drug concentrations directly to the tissue, and to utilize therapeutic agent in treatment that may not cross the blood brain barrier (BBB).

Convection-enhanced delivery can be chronic delivery, acute delivery, or a combination thereof. For example, the methods of treatment can include chronic delivery of radiolabeled nanoliposomes to a brain region, wherein the radiolabeled nanoliposomes are delivered at a continuous infusion rate over a time period of days, weeks, months or years. The methods of treatment can include acute delivery of radiolabeled nanoliposomes wherein the radiolabeled nanoliposomes are delivered in discrete boluses over the course of minutes or hours. The methods of treatment can include a combination of chronic and acute delivery wherein radiolabeled nanoliposomes are delivered at a continuous first infusion rate over a time period of days, weeks, months or years interspersed at regular or irregular intervals of limited duration infusions at a second, faster rate. Infusion rate and infusion time may be important factors in achieving adequate delivery. Additionally, infusion rate and infusion time may be a surrogate for infusion pressure. Methods of assessing the sufficiency of delivery include, but are not limited to, dosimetry, imaging, brain biopsy, treatment response assessment maps (TRAMs), and cerebral blood volume (CBV).

CED of a pharmaceutical composition or formulation comprising radiolabeled nanoliposomes, another therapeutic agent, or a combination thereof, can comprise an infusion rate of from about 0.1 L/min to about 20 μL/min.

CED of a pharmaceutical composition or formulation comprising radiolabeled nanoliposomes, another therapeutic agent, or a combination thereof, can comprise an infusion rate of greater than about: 0.1 μL/min, 0.5 μL/min, 0.7 μL/min, 1 μL/min, 1.2 μL/min, 1.5 μL/min, 1.7 μL/min, 2 μL/min, 2.2 μL/min, 2.5 μL/min, 2.7 μL/min, 3 μL/min, 3.5 μL/min, 4 μL/min, 5 μL/min, 6 μL/min, 7 μL/min, 7.5 μL/min, 10 μL/min, 15 μL/min, 20 μL/min, 25 μL/min, 30 μL/min, 35 μL/min, 40 μL/min, 45 μL/min, 50 μL/min, or higher than 50 μL/min.

CED of a pharmaceutical composition or formulation comprising radiolabeled nanoliposomes, another therapeutic agent, or a combination thereof, can comprise, or further comprise, an infusion rate of less than about: 50 μL/min, 45 μL/min, 40 μL/min, 35 μL/min, 30 μL/min, 25 μL/min, 20 μL/min, 15 μL/min, 12 μL/min, 10 μL/min, 7.5 μL/min, or 5 μL/min.

CED of a pharmaceutical composition or formulation comprising radiolabeled nanoliposomes, another therapeutic agent, or a combination thereof, can comprise, or further comprise incremental increases in flow rate, referred to as “stepping”, during delivery. Stepping can comprise infusion rates of between about 0.1 μL/min and about 20 μL/min. At periodic intervals, stepping CED can comprise one or more increases in infusion rate in steps of about: 0.1 μL/min, 0.2 μL/min, 0.3 μL/min, 0.4 μL/min, 0.5 μL/min, 0.6 μL/min, 0.7 μL/min, 0.8 μL/min, 0.9 μL/min, 1 μL/min, 1.25 μL/min, 1.5 μL/min, 2 μL/min, 2.5 μL/min, 3 μL/min or more.

The effective amount of radioactivity delivered by the radiolabeled liposome in a pharmaceutical composition or formulation administered by CED can be from 1 mCi to 1000 mCi. For example, the effective amount can be 1-1000 mCi, 1-500 mCi, 1-250 mCi, 1-150 mCi, 1-100 mCi, 1-75 mCi, 1-50 mCi, 1-25 mCi, 1-10 mCi, 1-5 mCi, 5-1000 mCi, 5-500 mCi, 5-250 mCi, 5-150 mCi, 5-100 mCi, 5-75 mCi, 5-50 mCi, 5-25 mCi, 5-10 mCi, 10-1000 mCi, 10-500 mCi, 10-250 mCi, 10-150 mCi, 10-100 mCi, 10-75 mCi, 10-50 mCi, 10-25 mCi, 25-1000 mCi, 25-500 mCi, 25-250 mCi, 25-150 mCi, 25-100 mCi, 25-75 mCi, 25-50 mCi, 50-1000 mCi, 50-500 mCi, 50-250 mCi, 50-150 mCi, 50-100 mCi, 50-75 mCi, 75-1000 mCi, 75-500 mCi, 75-250 mCi, 75-150 mCi, 75-100 mCi, 100-1000 mCi, 100-500 mCi, 100-250 mCi, 100-150 mCi, 150-1000 mCi, 150-500 mCi, 150-250 mCi, 250-1000 mCi, 250-500 mCi or 500-1000 mCi. In some embodiments, the effective amount is from 10 mCi to 250 mCi. In some embodiments the effective amount of radioactivity delivered by the radiolabeled liposome is from 50 mCi to 150 mCi. In some embodiments the effective amount of radioactivity delivered by the radiolabeled liposome is from 10 mCi to 500 mCi.

The effective amount of radioactivity delivered by the radiolabeled liposome administered by CED can be in a volume of 1-2000 μL, 1-1500 μL, 1-1000 μL, 1-750 μL, 1-500 μL, 1-250 μL, 1-100 μL, 1-50 μL, 1-10 μL, 10-2000 μL, 10-1500 μL, 10-1000 μL, 10-750 μL, 10-500 μL, 10-250 μL, 10-100 μL, 10-50 μL, 50-2000 μL, 50-1500 μL, 50-1000 μL, 50-750 μL, 50-500 μL, 50-250 μL, 50-100 μL, 100-2000 μL, 100-1500 μL, 100-1000 μL, 100-750 μL, 100-500 μL, 100-250 μL, 250-2000 μL, 250-1500 μL, 250-1000 μL, 250-750 μL, 250-500 μL, 500-2000 μL, 500-1500 μL, 500-1000 μL, 500-750 μL, 750-2000 μL, 750-1500 μL, 750-1000 μL, 1000-2000 μL, 1000-1500 μL, or 1500-2000 μL.

The effective amount of radioactivity delivered by the radiolabeled liposome (e.g., in a pharmaceutical composition) administered by CED can be in a concentration of about 0.5 mCi/ml, about 1 mCi/ml, about 1.5 mCi/ml, about 1.52 mCi/ml, about 2 mCi/ml, about 2.24 mCi/ml, about 2.36 mCi/ml about 2.5 mCi/ml, about 2.53 mCi/ml, about 2.54 mCi/ml, about 3 mCi/ml or about 3.5 mCi/ml.

For further teaching on the method of CED, see for example Saito et al., Exp. Neurol., 196:381-389, 2005; Krauze et al., Exp. Neurol., 196:104-111, 2005; Krauze et al., Brain Res. Brain Res. Protocol., 16:20-26, 2005; U.S. Patent Application Publication No. 2006/0073101; and U.S. Pat. No. 5,720,720, each of which is incorporated herein by reference in its entirety. See also Noble et al., Cancer Res. 2006 Mar. 1; 66(5):2801-6; Saito et al., J Neurosci Methods. 2006 Jun. 30; 154 (1-2):225-32; Hadaczek et al., Hum Gene Ther. 2006 March; 17(3):291-302; and Hadaczek et al., Mol Ther. 2006 July; 14(1):69-78, each of which is incorporated herein by reference in its entirety.

### Convection-Enhanced Delivery & Imaging

An advantage of convection-enhanced delivery can be the ability to use imaging technology to allow drug distribution to be seen during infusion. Gadolinium- and iodine-based imaging compounds can be used as tracers to safely and accurately track active ingredient distribution in real-time using, for example, magnetic resonance imaging or computed tomography imaging. These tracers can show the distribution of both small- and large-molecular-weight compounds with similar convective properties during infusion. Additionally, the radiolabeled nanoliposomes may be capable of imaging without the use of a tracer (e.g., by the radiolabeled nanoliposomes emission of gamma rays). In some embodiments, the methods and compositions provided herein do not comprise a tracing agent.

In methods that comprise delivering a pharmaceutical composition comprising radiolabeled nanoliposomes, another therapeutic agent, or a combination thereof, by CED, the pharmaceutical composition can comprise a tracing agent. The tracing agent can enable monitoring the distribution of the tracing agent as it moves through the CNS, and ceasing delivery of the pharmaceutical composition when the radiolabeled nanoliposomes, another therapeutic agent, or a combination thereof is distributed in a predetermined volume within the CNS. The movement of the tracing agent through the solid tissue can be monitored by an imaging technique such as magnetic resonance imaging (MRI), computed tomography imaging, X-ray computed tomography (CT), or single-photon emission computerized tomography. The tracing agent can have a mobility in CNS tissue that is substantially similar to the therapeutic agent (e.g., radiolabeled nanoliposomes), and delivery can be ceased when the tracing agent is observed to reach a desired region or achieve a desired volume of distribution, or to reach or nearly reach or exceed the borders of the target tissue. In some embodiments, the radiolabeled nanoparticles of the present disclosure are capable of providing the same functionality (e.g., monitoring distribution) as a composition comprising a tracer.

The desired volume may correspond to a particular region of the brain that is targeted for therapy. The desired volume of distribution can be “substantially similar” to the volume of distribution observed for a tracing agent that is being monitored to follow the infusion. “Substantially similar” refers to a difference in volume of less than 20%. More preferably, the difference in volume is less than 15%, more preferably less than 10%, more preferably less than 5%. By monitoring the distribution of the tracing agent, infusion may be ceased when the predetermined volume of distribution is reached.

The desired volume of distribution can be determined, for example, by using imaging software that is standard in the art, e.g., iFLOW™. See also, for example, Krautze et al., Brain Res. Protocols, 16:20-26, 2005; and Saito et al., Exp. Neurol., 196:3891-389, 2005, each of which is incorporated herein by reference in its entirety.

The tracer can comprise a paramagnetic ion for use with MRI. Suitable metal ions include those having atomic numbers of 22-29 (inclusive), 42, 44 and 58-70 (inclusive) and have oxidation states of +2 or +3. Examples of such metal ions are chromium (III), manganese (II), iron (II), iron (III), cobalt (II), nickel (II), copper (II), praseodymium (III), neodymium (III), samarium (III), gadolinium (III), terbium (III), dysprosium (III), holmium (III), erbium (III) and ytterbium (III).

In embodiments wherein X-ray imaging (such as CT) is used to monitor CED, the tracer may comprise a radiopaque material. Suitable radiopaque materials include, but are not limited to, iodine compounds, barium compounds, gallium compounds, thallium compounds, and the like. Specific examples of radiopaque materials include barium, diatrizoate, ethiodized oil, gallium citrate, iocarmic acid, iocetamic acid, iodamide, iodipamide, iodoxamic acid, iogulamide, iohexol, iopamidol, iopanoic acid, ioprocemic acid, iosefamic acid, ioseric acid, iosulamide meglumine, iosumetic acid, iotasul, iotetric acid, iothalamic acid, iotroxic acid, ioxaglic acid, ioxotriroic acid, ipodate, meglumine, metrizamide, metrizoate, propyliodone, and thallous chloride.

### Devices for Convection-Enhanced Delivery (CED)

It is contemplated that any suitable device can be used in the methods disclosed herein wherein a pharmaceutical composition or formulation comprising radiolabeled nanoliposomes, another therapeutic agent, or a combination thereof, is administered to a brain region by CED.

A delivery device can comprise a pump that is capable of delivering a pharmaceutical composition or formulation comprising radiolabeled nanoliposomes, another therapeutic agent, or a combination thereof, by CED. The pump can be an osmotic pump or an infusion pump. These pumps may be useful in preventing undesired issues associated pump malfunctions, such as issues associated with back-pressure and/or occlusion, because these pump may comprise a back-pressure sensor used to force shut down if back-pressure rises above a threshold value. The device can comprise, or can be used in conjunction with, a catheter or cannula that facilitates localized delivery to a brain region of a subject. The catheter or cannula can comprise multiple outlet ports. The catheter or cannula can comprise an outer tubing to provide structural rigidity to the catheter or cannula. The catheter or cannula can be a reflux-free step-design cannula.

One or more catheters or cannuli can be inserted into or near one or more brain regions of a subject. Stereotactic maps and positioning devices are available, for example from ASI Instruments, Warren, Mich. Positioning can be conducted by using anatomical maps obtained by CT and/or MRI imaging of the subject's brain to help guide the injection device to the chosen target.

### Reflux-Free Step-Design Cannula

CED can be performed with the use of a CED-compatible reflux-free step-design cannula, such as that disclosed in Krauze et al., J Neurosurg. 2005 November; 103(5):923-9, incorporated herein by reference in its entirety, as well as in U.S. Patent Application Publication No. US 2007/0088295 A1, incorporated herein by reference in its entirety, and U.S. Patent Application Publication No. US 2006/0135945 A1, incorporated herein by reference in its entirety.

In one embodiment, the step-design cannula is compatible with chronic administration. In another embodiment, the step-design cannula is compatible with acute administration. In another embodiment, the step-design cannula is compatible with a combination of chronic administration interspersed with periods of acute administration at a higher infusion rate or with a higher level of the therapeutic agent.

### Alternative Catheters

CED can be performed with the use of a neurosurgical apparatus comprising a CED-compatible catheter, such as that disclosed in WO 2013/127884, incorporated herein by reference in its entirety.

The neurosurgical apparatus can comprise a guide device and a catheter. The guide device can comprise an elongated tube having a head at its proximal end. In use, the elongated tube can be inserted into the brain towards a target brain region via a hole formed in the skull. The head can be used to securely attach the guide device to the skull. This insertion may be performed using a stereoguide or surgical robot based technique. An internal channel is provided through the head and bore of the tube. The catheter can then be passed down this channel and into the brain in the vicinity of the selected target.

This disclosure provides for delivery kits or devices comprising a pump that is capable of effecting delivery of a pharmaceutical composition or formulation comprising radiolabeled nanoliposomes (e.g., ¹⁸⁶Re nanoliposomes) (e.g., such as when a pharmaceutical composition comprises the radiolabeled nanoliposome), another therapeutic agent, or a combination thereof, by CED. The kits or devices further comprise a pharmaceutical composition comprising radiolabeled nanoliposomes, another therapeutic agent, or a combination thereof, such as any of those disclosed herein. The kits or devices further comprises a CED-compatible cannula or catheter. The cannula or catheter can be compatible with chronic or acute administration.

### Radiation

In some embodiments, provided herein are pharmaceutical compositions for delivering radiation. In some embodiments, the pharmaceutical composition comprises a liposome. In some embodiments, the liposome comprises a nanoliposome. In some embodiments, the pharmaceutical composition comprises isotopic rhenium. In some embodiments, isotopic rhenium comprises 186-rhenium (¹⁸⁶Re) In some embodiments, the pharmaceutical composition comprises a liposome (e.g., nanoliposome) and isotopic rhenium (e.g., ¹⁸⁶Re). In some embodiments, the pharmaceutical composition comprises a compound of Formula I and a liposome. In some embodiments, the pharmaceutical composition comprises ¹⁸⁶Re nanoliposomes, comprising isotopic rhenium (e.g., ¹⁸⁶Re) and nanoliposomes.

In some embodiments, the pharmaceutical composition delivers radiation. In some embodiments, the pharmaceutical composition delivers β-rays. In some embodiments, the pharmaceutical composition delivers gamma rays. In some embodiments, the pharmaceutical composition delivers one type of radiation. pharmaceutical composition delivers two types of radiation (e.g., β-rays and gamma rays). In some embodiments, provided herein are pharmaceutical compositions (e.g., comprising a liposome (e.g., nanoliposome) and isotopic (e.g., 186) rhenium) for delivering radiation (e.g., two types of radiation) (e.g., β-rays and/or gamma rays).

In some embodiments, the pharmaceutical composition is administered to a subject. In some embodiments, the subject is a patient in need of treatment. In some embodiments, the patient in need of treatment comprises cancer.

Radiation, in particular β-rays, may be useful for treatment of a patient comprising a cancer. Gamma-rays may be useful for visualizing purposes. In some embodiments, visualizing comprises imaging. A pharmaceutical composition that delivers both gamma-rays and β-rays may be beneficial for delivering therapy (e.g., radiation for the treatment of a cancer) and for allowing visualization of the delivered therapy. For example, gamma rays may have adequate energy for visualization (e.g., imaging) during delivery of therapy (e.g., during administration of ¹⁸⁶Re nanoliposomes) and for biodistribution assessment. Imaging using gamma rays may be beneficial for modifying one or more delivery parameters in order to dose a subject.

By delivering both β-rays and gamma rays, one or more delivery parameters of the therapy may be adjusted to allow for modifications to therapy. In some embodiments, the pharmaceutical composition is delivered and visualized (e.g., imaged). In some embodiments, the pharmaceutical composition is delivered and visualized simultaneously. In some embodiments, the pharmaceutical composition is delivered and later visualized. In some embodiments, the pharmaceutical composition is delivered (e.g., by CED), visualized (e.g., simultaneously), and a modification is made to one or more delivery parameters. In some embodiments, the modification to the one or more delivery parameters occurs during delivery (e.g., convection) of the pharmaceutical composition. In some embodiments, the modification to the one or more delivery parameters occurs after delivery of the pharmaceutical composition.

In some embodiments, modifications to therapy (e.g., one or more delivery parameters) are made in response to delivery and visualization (e.g., imaging) of the pharmaceutical composition. In some embodiments, the one or more delivery parameters comprise an infusion rate, volume amount, concentration(s), location(s) of administration, number of catheters, and/or the device being used for delivery of the pharmaceutical composition. Any of the volumes, radiation doses, or doses of the pharmaceutical compositions disclosed herein may be sufficient to deliver β-rays and gamma rays.

In some embodiments, delivery of the pharmaceutical composition (e.g., ¹⁸⁶Re nanoliposomes delivering β-rays and gamma rays) allows for therapy to be monitored to ensure successful delivery of the pharmaceutical composition to the target (e.g., tumor).

### Imaging

Gamma-rays may be beneficial for visualizing (e.g., viewing) purposes. In some embodiments, visualizing comprises imaging. For example, the pharmaceutical composition may deliver a sufficient dose of β-rays to allow for the treatment of cancer, and a sufficient dose of gamma rays to allow for imaging (e.g., in vivo imaging). In some embodiments, visualizing comprises imaging. In some embodiments, the composition further comprises a tracer. In some embodiments, the pharmaceutical composition does not comprise a tracer. In some cases, the gamma rays are sufficient for visualization without the use of a tracer.

In some embodiments, imaging comprises single-photon emission computed tomography-computed tomography (SPECT-CT). In some embodiments imaging comprises single-photon emission computed tomography (SPECT). In some embodiments, imaging comprises computed tomography (CT). In some embodiments, imaging comprises magnetic resonance imaging (MRI). In some embodiments, imaging may comprise alternative forms of imaging, such as radiography (e.g., X-rays), fluoroscopy, nuclear medicine, positron emission tomography (PET) (e.g., single-photon emission computed tomography), computed tomography (CT), intraoperative imaging, and mammography, or a combination of thereof. In some embodiments, imaging comprises planar imaging. In some embodiments, imaging comprises a combination of any of the imaging techniques disclosed herein (e.g., SPECT and MRI). In some embodiments, imaging comprises a static image. In some embodiments, imaging comprises a dynamic image.

### Methods of Treatment

Provided herein, in some embodiments, are methods of using the radiolabeled liposome disclosed herein, which may be incorporated into any of the pharmaceutical compositions disclosed herein.

Provided herein, in one aspect, is a method of treating a disease or disorder in a subject in need thereof, comprising administering a therapeutically effective amount of a radiolabeled liposome comprising a liposome (e.g., nanoliposome) and a compound of Formula I:

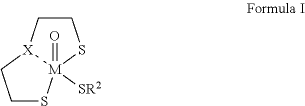

or a pharmaceutically acceptable salt thereof, wherein:
   M is ^{99m}Tc, ¹⁸⁶Re, ¹⁸⁸Re, or a combination thereof;
   X is NR¹;
   R¹ is CH₂CH₂NEt₂ or CH₂CH₂CH₂CH₃; and
   R² is CH₂CH₂N(CH₂CH₂SH)(CH₂CH₂NEt₂) or
CH₂CH₂N(CH₂CH₂SH)(CH₂CH₂CH₂CH₃).

In some embodiments, R¹ is CH₂CH₂NEt₂. In some embodiments, R¹ is CH₂CH₂CH₂CH₃. In some embodiments, R² is CH₂CH₂N(CH₂CH₂SH)(CH₂CH₂NEt₂). In some embodiments, R² is CH₂CH₂N(CH₂CH₂SH)(CH₂CH₂CH₂CH₃). In some embodiments, R¹ is CH₂CH₂NEt₂ and R² is CH₂CH₂N(CH₂CH₂SH)(CH₂CH₂NEt₂). In some embodiments, R¹ is CH₂CH₂CH₂CH₃ and R² is CH₂CH₂N(CH₂CH₂SH)(CH₂CH₂CH₂CH₃).

In some embodiments, M is ^{99m}Tc. In some embodiments, M is ¹⁸⁶Re. In some embodiments, M is ¹⁸⁸Re. In some embodiments, when M is ¹⁸⁶Re and the liposomes comprise nanoliposomes the radiolabeled liposome is referred to as ¹⁸⁶Re nanoliposomes.

In some embodiments, the compound is incorporated or attached to the liposome.

In some embodiments, the liposome further comprises a drug that is incorporated within the liposome.

In some embodiments, the drug is a compound comprising at least one thiol group. In some embodiments, the drug reacts with the compound. In some embodiments, the drug comprises glutathione, cysteine, N-acetyl cysteine, 2-mercaptosuccinic acid, 2,3-dimercaptosuccinic acid, captopril, or a combination thereof. In some embodiments, the drug comprises glutathione. In some embodiments, the drug comprises cysteine. In some embodiments, the drug comprises N-acetyl cysteine. In some embodiments, the drug comprises 2-mercaptosuccinic acid. In some embodiments, the drug comprises 2,3-dimercaptosuccinic acid. In some embodiments, the drug comprises captopril. In some embodiments, the drug comprises a combination.

In some embodiments, the liposome comprises a lipid. In some embodiments, the liposome comprises a phospholipid.

In some embodiments, the liposome comprises a cholesterol or a cholesterol analogue. In some embodiments, the liposome comprises a cholesterol. In some embodiments, the liposome comprises a cholesterol analogue. In some embodiments, the liposome comprises distearoyl phosphatidylcholine.

In some embodiments, the radiolabeled liposome (e.g., nanoliposome) comprises from about 0.01 mCi to about 400 mCi of the compound per 50 mg of lipid used to prepare the liposome. In some embodiments, the radiolabeled liposome comprises from about 1 mCi to about 200 mCi of the compound per 50 mg of lipid used to prepare the liposome. In some embodiments, the radiolabeled liposome comprises from about 10 mCi to about 100 mCi of the compound per 50 mg of lipid used to prepare the liposome. In some embodiments, the radiolabeled liposome comprises from about 25 mCi to about 50 mCi of the compound per 50 mg of lipid used to prepare the liposome. In some embodiments, the radiolabeled liposome comprises about 1.5 mCi of the compound per 50 mg of lipid used to prepare the liposome. In some embodiments, the radiolabeled liposome comprises about 2.5 mCi of the compound per 50 mg of lipid used to prepare the liposome.

In some embodiments, the liposome (e.g., nanoliposome) further comprises a chemotherapeutic agent, an antibiotic agent, or a treatment molecule, wherein the chemotherapeutic agent, the antibiotic agent, or the treatment molecule is incorporated or attached to the liposome. In some embodiments, the liposome further comprises a chemotherapeutic agent. In some embodiments, the liposome further comprises an antibiotic agent. In some embodiments, the liposome further comprises a treatment molecule.

In some embodiments, the disease or disorder is cancer. In some embodiments, the cancer is selected from lung cancer, breast cancer, colorectal cancer, prostate cancer, skin cancer, stomach cancer, bladder cancer, liver cancer, leukemia, lymphoma, ovarian cancer, pancreatic cancer, hepatocellular carcinoma, melanoma, sarcoma, head and neck cancer, glioma, glioblastoma, medulloblastoma, ependymoma, diffuse intrinsic pontine glioma, leptomeningeal metastases, and pediatric high-grade glioma. In some embodiments, the cancer is lung cancer. In some embodiments, the cancer is breast cancer. In some embodiments, the cancer is colorectal cancer. In some embodiments, the cancer is prostate cancer. In some embodiments, the cancer is skin cancer. In some embodiments, the cancer is stomach cancer. In some embodiments, the cancer is bladder cancer. In some embodiments, the cancer is liver cancer. In some embodiments, the cancer is leukemia. In some embodiments, the cancer is lymphoma. In some embodiments, the cancer is ovarian cancer. In some embodiments, the cancer is pancreatic cancer. In some embodiments, the cancer is hepatocellular carcinoma. In some embodiments, the cancer is melanoma. In some embodiments, the cancer is sarcoma. In some embodiments, the cancer is head and neck cancer. In some embodiments, the cancer is glioma. In some embodiments, the cancer is glioblastoma. In some embodiments, the glioblastoma is recurrent glioblastoma. In some embodiments, the cancer is leptomeningeal metastases. In some embodiments, the cancer is medulloblastoma. In some embodiments, the cancer is ependymoma. In some embodiments, the cancer is diffuse intrinsic pontine glioma. In some embodiments, the cancer is pediatric high-grade glioma.

In some embodiments, the subject has previously received treatment comprising bevacizumab. In some embodiments, the subject has not previously received treatment comprising bevacizumab.

In some embodiments, the pharmaceutical composition (e.g., comprising radiolabeled nanoliposomes) is an infusate. In some embodiments, the radiolabeled nanoliposomes are administered via infusion as an infusate comprising the radiolabeled liposomes. In some embodiments, infusion comprises infusion via ventricular reservoir.

In some embodiments, the radiolabeled liposome (e.g., in a pharmaceutical composition) is administered via convection-enhanced delivery. In some embodiments, the convection-enhanced delivery comprises administration of the radiolabeled liposome via one or more catheters. In some embodiments, the convection-enhanced delivery comprises administration of the radiolabeled liposome via one catheter. In some embodiments, the convection-enhanced delivery comprises administration of the radiolabeled liposome via two catheters. In some embodiments, the convection-enhanced delivery comprises administration of the radiolabeled liposome via three catheters. In some embodiments, the convection-enhanced delivery comprises administration of the radiolabeled liposome via four catheters. In some embodiments, the convection-enhanced delivery comprises administration of the radiolabeled liposome via more than four catheters.

In some embodiments, the infusate is administered with a maximum flow rate of from about 1 μL min⁻¹ to about 50 μL min⁻¹. In some embodiments, the infusate is administered with a maximum flow rate of from about 5 μL min⁻¹ to about 20 μL min⁻¹. In some embodiments, the infusate is administered with a maximum flow rate of about 1 μL min⁻¹. In some embodiments, the infusate is administered with a maximum flow rate of about 2 μL min⁻¹. In some embodiments, the infusate is administered with a maximum flow rate of about 5 μL min⁻¹. In some embodiments, the infusate is administered with a maximum flow rate of about 10 μL min⁻¹. In some embodiments, the infusate is administered with a maximum flow rate of about 15 μL min⁻¹. In some embodiments, the infusate is administered with a maximum flow rate of about 20 μL min⁻¹. In some embodiments, the infusate is administered with a maximum flow rate of about 25 μL min⁻¹. In some embodiments, the infusate is administered with a maximum flow rate of more than about 25 μL min⁻¹. In some embodiments, the infusate is administered with a maximum flow rate of about 30 μL min⁻¹. In some embodiments, the infusate is administered with a maximum flow rate of about 35 μL min⁻¹. In some embodiments, the infusate is administered with a maximum flow rate of about 40 μL min⁻¹. In some embodiments, the infusate is administered with a maximum flow rate of about 45 μL min⁻¹. In some embodiments, the maximum flow rate is a maximum flow rate per catheter.

In some embodiments, the infusate is administered with an exit velocity from about 0.001 mm/min to about 1.0 mm/min. In some embodiments, the infusate is administered with an exit velocity from about 0.001 mm/min to about 0.1 mm/min. In some embodiments, the infusate is administered with an exit velocity from about 0.001 mm/min to about 0.5 mm/min. In some embodiments, the infusate is administered with an exit velocity from about 0.1 mm/min to about 1.0 mm/min. In some embodiments, the infusate is administered with an exit velocity from about 0.5 mm/min to about 1.0 mm/min. In some embodiments, the infusate is administered with an exit velocity of about 0.009, 0.022, 0.045, 0.067, or 0.090 mm/min. In some embodiments, the infusate is administered with an exit velocity of about 0.081, 0.201, 0.403, 0.604, or 0.806 mm/min. In some embodiments, the infusate is administered with an exit velocity of about 0.020, 0.050, 0.101, 0.151, or 0.201 mm/min.

In some embodiments, administration of the pharmaceutical composition comprises one catheter, two catheters, three catheters or four catheters.

In some embodiments, the rate of infusate delivery per catheter is 1 ul/min, 2 ul/min, 3 ul/min, 4 ul/min, 5 ul/min, 6 ul/min, 7 ul/min, 8 ul/min, 9 ul/min, 10 ul/min, 11 ul/min, 12 ul/min, 13 ul/min, 14 ul/min, 15 ul/min, 16 ul/min, 17 ul/min, 18 ul/min, 19 ul/min, 20 ul/min, 30 ul/min, 35 ul/min or about 40 ul/min.

In some embodiments, the minimum interval of administration is about 5 min, about 10 min, about 15 min, about 20 min, about 25 min, about 30 min, about 40 min, about 50 min, about 60 min, about 90 minutes, about 120 minutes or about 150 minutes.

In some embodiments, the amount of radioactivity delivered by the radiolabeled liposome (e.g., radiolabeled nanoliposome in a pharmaceutical composition) is from about 0.1 mCi to about 50 mCi. In some embodiments, the amount of radioactivity delivered by the radiolabeled liposome is from about 1 mCi to about 40 mCi. In some embodiments, the amount of radioactivity delivered by the radiolabeled liposome is from about 1 mCi to about 30 mCi. In some embodiments, the amount of radioactivity delivered by the radiolabeled liposome is from about 1 mCi to about 20 mCi. In some embodiments, the amount of radioactivity delivered by the radiolabeled liposome is from about 10 mCi to about 30 mCi. In some embodiments, the amount of radioactivity delivered by the radiolabeled liposome is from about 20 mCi to about 30 mCi. In some embodiments, the amount of radioactivity delivered by the radiolabeled liposome is about 1 mCi. In some embodiments, the amount of radioactivity delivered by the radiolabeled liposome is about 2 mCi. In some embodiments, the amount of radioactivity delivered by the radiolabeled liposome is about 3 mCi. In some embodiments, the amount of radioactivity delivered by the radiolabeled liposome is about 4 mCi. In some embodiments, the amount of radioactivity delivered by the radiolabeled liposome is about 5 mCi. In some embodiments, the amount of radioactivity delivered by the radiolabeled liposome is about 6 mCi. In some embodiments, the amount of radioactivity delivered by the radiolabeled liposome is about 7 mCi. In some embodiments, the amount of radioactivity delivered by the radiolabeled liposome is about 8 mCi. In some embodiments, the amount of radioactivity delivered by the radiolabeled liposome is about 9 mCi. In some embodiments, the amount of radioactivity delivered by the radiolabeled liposome is about 10 mCi. In some embodiments, the amount of radioactivity delivered by the radiolabeled liposome is about 11 mCi. In some embodiments, the amount of radioactivity delivered by the radiolabeled liposome is about 12 mCi. In some embodiments, the amount of radioactivity delivered by the radiolabeled liposome is about 13 mCi. In some embodiments, the amount of radioactivity delivered by the radiolabeled liposome is about 13.4 mCi. In some embodiments, the amount of radioactivity delivered by the radiolabeled liposome is about 14 mCi. In some embodiments, the amount of radioactivity delivered by the radiolabeled liposome is about 15 mCi. In some embodiments, the amount of radioactivity delivered by the radiolabeled liposome is about 16 mCi. In some embodiments, the amount of radioactivity delivered by the radiolabeled liposome is about 17 mCi. In some embodiments, the amount of radioactivity delivered by the radiolabeled liposome is about 18 mCi. In some embodiments, the amount of radioactivity delivered by the radiolabeled liposome is about 19 mCi. In some embodiments, the amount of radioactivity delivered by the radiolabeled liposome is about 20 mCi. In some embodiments, the amount of radioactivity delivered by the radiolabeled liposome is about 21 mCi. In some embodiments, the amount of radioactivity delivered by the radiolabeled liposome is about 22 mCi. In some embodiments, the amount of radioactivity delivered by the radiolabeled liposome is about 22.3 mCi. In some embodiments, the amount of radioactivity delivered by the radiolabeled liposome is more than about 22.3 mCi. In some embodiments, the amount of radioactivity delivered by the radiolabeled liposome is about 23 mCi. In some embodiments, the amount of radioactivity delivered by the radiolabeled liposome is about 24 mCi. In some embodiments, the amount of radioactivity delivered by the radiolabeled liposome is about 25 mCi. In some embodiments, the amount of radioactivity delivered by the radiolabeled liposome is about 26 mCi. In some embodiments, the amount of radioactivity delivered by the radiolabeled liposome is about 27 mCi. In some embodiments, the amount of radioactivity delivered by the radiolabeled liposome is about 28 mCi. In some embodiments, the amount of radioactivity delivered by the radiolabeled liposome is about 29 mCi. In some embodiments, the amount of radioactivity delivered by the radiolabeled liposome is about 30 mCi. In some embodiments, the amount of radioactivity delivered by the radiolabeled liposome is about 31 mCi. In some embodiments, the amount of radioactivity delivered by the radiolabeled liposome is about 31.2 mCi. In some embodiments, the amount of radioactivity delivered by the radiolabeled liposome is more than about 31.2 mCi. In some embodiments, the amount of radioactivity delivered by the radiolabeled liposome is about 35 mCi. In some embodiments, the amount of radioactivity delivered by the radiolabeled liposome is about 40 mCi. In some embodiments, the amount of radioactivity delivered by the radiolabeled liposome is about 41.5 mCi. In some embodiments, the amount of radioactivity delivered by the radiolabeled liposome is more than about 41.5 mCi. In some embodiments, the amount of radioactivity delivered by the radiolabeled liposome is about 45 mCi. In some embodiments, the amount of radioactivity delivered by the radiolabeled liposome is more than about 45 mCi.

In some embodiments, the volume of infusate is from about 0.1 mL to about 25 mL. In some embodiments, the volume of infusate is from about 0.5 mL to about 10 mL. In some embodiments, the volume of infusate is about 0.66 mL. In some embodiments, the volume of infusate is about 1 mL. In some embodiments, the volume of infusate is about 1.32 mL. In some embodiments, the volume of infusate is about 2 mL. In some embodiments, the volume of infusate is about 2.64 mL. In some embodiments, the volume of infusate is about 3 mL. In some embodiments, the volume of infusate is about 4 mL. In some embodiments, the volume of infusate is about 5 mL. In some embodiments, the volume of infusate is about 5.28 mL. In some embodiments, the volume of infusate is about 6 mL. In some embodiments, the volume of infusate is about 7 mL. In some embodiments, the volume of infusate is about 8 mL. In some embodiments, the volume of infusate is about 8.8 mL. In some embodiments, the volume of infusate is more than about 8.8 mL. In some embodiments, the volume of infusate is about 9 mL. In some embodiments, the volume of infusate is about 10 mL. In some embodiments, the volume of infusate is about 11 mL. In some embodiments, the volume of infusate is about 12 mL. In some embodiments, the volume of infusate is about 12.3 mL. In some embodiments, the volume of infusate is about 13 mL. In some embodiments, the volume of infusate is about 13.2 mL. In some embodiments, the volume of infusate is about 14 mL. In some embodiments, the volume of infusate is about 15 mL. In some embodiments, the volume of infusate is about 16 mL. In some embodiments, the volume of infusate is about 16.35 mL. In some embodiments, the volume of infusate is about 17 mL. In some embodiments, the volume of infusate is about 18 mL. In some embodiments, the volume of infusate is about 18.5 mL. In some embodiments, the volume of infusate is more than about 18.5 mL. In some embodiments, the volume of infusate is delivered to a single hemisphere of the brain (e.g., comprising glioblastoma). In some embodiments, the volume of infusate is delivered to both hemispheres of the brain (e.g., comprising glioblastoma).

In some embodiments, the distribution volume is about 2 mL, about 2.64 mL, about 5 mL, about 5.28 mL, about 10 mL, about 10.5 mL, about 13 mL, about 13.2 mL, about 19 mL, about 19.4 mL, about 26 mL, about 26.4 mL, about 33 mL, about 33.3 mL, or about 35 mL.

In some embodiments, the amount of radioactivity delivered by the radiolabeled liposome per volume of infusate is from about 0.1 mCi mL⁻¹ to about 50 mCi mL⁻¹. In some embodiments, the amount of radioactivity delivered by the radiolabeled liposome per volume of infusate is from about 0.5 mCi mL⁻¹ to about 10 mCi mL⁻¹. In some embodiments, the amount of radioactivity delivered by the radiolabeled liposome per volume of infusate is from about 1 mCi mL⁻¹ to about 5 mCi mL⁻¹. In some embodiments, the amount of radioactivity delivered by the radiolabeled liposome per volume of infusate is about 1 mCi mL⁻¹. In some embodiments, the amount of radioactivity delivered by the radiolabeled liposome per volume of infusate is about 1.5 mCi mL⁻¹. In some embodiments, the amount of radioactivity delivered by the radiolabeled liposome per volume of infusate is about 2 mCi mL⁻¹. In some embodiments, the amount of radioactivity delivered by the radiolabeled liposome per volume of infusate is about 2.24 mCi mL⁻¹. In some embodiments, the amount of radioactivity delivered by the radiolabeled liposome per volume of infusate is about 2.36 mCi mL⁻¹. In some embodiments, the amount of radioactivity delivered by the radiolabeled liposome per volume of infusate is about 2.5 mCi mL⁻¹. In some embodiments, the amount of radioactivity delivered by the radiolabeled liposome per volume of infusate is about 2.53 mCi mL⁻¹. In some embodiments, the amount of radioactivity delivered by the radiolabeled liposome per volume of infusate is about 2.54 mCi mL⁻¹. In some embodiments, the amount of radioactivity delivered by the radiolabeled liposome per volume of infusate is more than about 2.54 mCi mL⁻¹.

In some embodiments, the amount of absorbed dose (e.g., radiation dose absorbed) is about 10 Gy, about 20 Gy, about 30 Gy, about 40 Gy, about 50 Gy, about 80 Gy, about 100 Gy, about 120 Gy, about 150 Gy, about 200 Gy, about 225 Gy, about 250 Gy, about 300 Gy, about 350 Gy, about 400 Gy, about 425 Gy, about 450 Gy, about 500 Gy, about 550 Gy, about 575 Gy, about 600 Gy, about 625 Gy, about 650 Gy, about 675 Gy, about 700 Gy, about 740 Gy, about 750 Gy, or about 800 Gy. In some embodiments, the amount of absorbed dose is less than 100 Gy. In some embodiments, the amount of absorbed dose is greater than 100 Gy.

In some embodiments, the method further comprises imaging the radiolabeled liposome concomitant with administration. In some embodiments, the method further comprises imaging the radiolabeled liposome subsequent to administration. In some embodiments, the method results in sustained intratumoral accumulation.

### Delivering Increments of New Radiation

In some embodiments, provided herein are methods of delivering increments of radiation to a subject. In some embodiments, the methods comprise delivering increments of radiation to a subject who previously received a dose of radiation therapy. In some embodiments, the previously received dose of radiation therapy was a maximum dose of radiation therapy. In some embodiments, the increments of radiation comprise β-rays. In some embodiments, the subject comprises a patient comprising a cancer. In some embodiments, an increment comprises an additional portion of radiation.

In some embodiments, the methods provided herein comprise delivering an additional portion of radiation (e.g., β-rays) to a subject in need thereof (e.g., a patient comprising a cancer) by delivering any of the pharmaceutical compositions provided herein. In some embodiments, an increment comprises an amount of radiation greater than an amount of radiation received in the immediately preceding treatment. In some embodiments, an increment comprises the same amount of radiation received in a previous treatment (e.g., a treatment received before the current treatment). In some embodiments, delivering increments of radiation comprises delivering varying amounts of radiation by modifying one or more delivery parameters (e.g., volume, dose, infusion rate, infusion time, etc.) of the pharmaceutical compositions provided herein. In some embodiments, delivering modified amounts of radiation comprises altering the volume of infusate and/or the dose of radiation.

In some embodiments, delivering increments of radiation comprises delivering a larger dose of radiation than previously received (e.g., previously received from the pharmaceutical compositions disclosed herein). In some embodiments, the increment comprises any of the doses or volumes provided herein. In some embodiments, the increment comprises a portion of a dose. In some embodiments, delivering an increment of radiation comprises delivering an amount of radiation that is greater than previously received (e.g., in reference to the current round of treatment or in reference to receiving radiation from the pharmaceutical compositions disclosed herein). The increments may be any of the doses disclosed throughout the application, including but not limited to, those doses disclosed in the Examples (e.g., Example 4).

In some embodiments, delivering increments of radiation comprises delivering β-rays. In some embodiments, delivering increments of radiation comprises delivering gamma rays. In some embodiments, delivering increments of radiation comprises delivering β-rays and gamma rays.

In some embodiments, the radiation comprises new radiation. In some embodiments new radiation comprises radiation (e.g., β-rays) and/or a pharmaceutical composition not received by the subject before receiving any of the pharmaceutical compositions disclosed herein.

In some embodiments, the method comprises delivering increments of radiation to a subject (e.g., patient comprising a cancer) who previously received radiation. In some embodiments, delivering increments comprises delivering increasingly larger doses of radiation.

In some embodiments, the subject receiving increments of radiation previously received a maximum dose of radiation. In some embodiments, the subject receiving increments of radiation previously received a maximum dose of radiation for the treatment of their primary and/or secondary disease(s). In some embodiments, the subject previously received a maximum dose of radiation for treatment of a cancer (e.g., glioblastoma). In some cases, the maximum dose of radiation comprises a maximum permissible dose (e.g., an upper limit of allowed radiation that a subject may receive without the risk of significant side effects).

In some embodiments, the subject has not received treatment for treatment of a disease (e.g., cancer) or condition before. In some embodiments, the subject has not received radiation treatment for treatment of a disease (e.g., cancer) or condition before. In some embodiments, the subject has previously received a non-radiation therapy (e.g., bevacizumab) for treatment of the disease (e.g., cancer) or condition. In some embodiments, the subject has not previously received a non-radiation therapy for the treatment of the disease. In some embodiments, the subject previously received radiation therapy comprising β-rays from a pharmaceutical composition other than those disclosed herein for the treatment of the disease. In some embodiments, the subject has received radiation therapy comprising β-rays from a pharmaceutical composition disclosed herein for the treatment of the disease. In some embodiments, delivering the increments of radiation comprises delivering the increments of radiation to a subject who previously received a prior maximum dose of therapy (e.g., radiation therapy) for a treatment (e.g., treatment of their primary and/or secondary disease).

### Administration and Visualization

In some embodiments, provided herein, are methods of delivering and visualizing (e.g., imaging) radiotherapeutic delivery. In some embodiments, the radiotherapeutic comprises the pharmaceutical composition disclosed herein. In some embodiments, provided herein, are methods of delivering and visualizing (e.g., imaging) the pharmaceutical composition (e.g., the radiolabeled liposome (e.g., ¹⁸⁶Re nanoliposome))) delivery. In some embodiments, the method comprises ensuring delivery to the target. In some embodiments, the target is a cancer. In some embodiments, the target is a tumor. In some embodiments, the tumor is a primary tumor. In some embodiments, the tumor is a secondary tumor. In some embodiments, the method comprises modifying (e.g., making changes to) one or more delivery parameters during delivery. In some embodiments, delivering (e.g., by CED) the radiotherapeutic (e.g., the pharmaceutical composition). In some embodiments, the method comprises modifying one or more delivery parameters during delivery and ensuring delivery of the pharmaceutical composition to the target during delivery.

In some embodiments, visualizing (e.g., imaging) occurs before administering (e.g., by CED) the pharmaceutical composition. In some embodiments, the method comprises visualizing delivery of the pharmaceutical composition (e.g., radiotherapy (e.g., the pharmaceutical composition) (e.g., radiolabeled liposome (e.g., ¹⁸⁶Re))) during delivery of the pharmaceutical composition. Concurrent (e.g., simultaneous or real-time) administration (e.g., by CED) and visualization may allow for real-time modifications to be made to one or more delivery parameters. Furthermore, concurrent administration and visualization may be helpful for ensuring delivery of the pharmaceutical composition to the target (e.g., tumor). Visualizing (e.g., imaging) and adjusting one or more delivery parameters concurrently (e.g., simultaneously) may be useful for ensuring safety and/or efficacy. In some embodiments, visualizing occurs after administering (e.g., by CED) the pharmaceutical composition.

The gamma-rays may enable monitoring the distribution of the pharmaceutical composition as it moves through the CNS, and ceasing delivery of the pharmaceutical composition when the radiolabeled nanoliposomes, another therapeutic agent, or a combination thereof is distributed in a predetermined volume within the CNS. Visualizing the gamma-rays may help ensure cessation of delivery of the pharmaceutical composition when the gamma rays are observed to reach a desired region or achieve a desired volume of distribution, or to reach or nearly reach or exceed the borders of the target tissue.

In some embodiments, the method comprising administering (e.g., by CED) and visualizing (e.g., simultaneously) the pharmaceutical composition (e.g., comprising the radiotherapeutic) further comprises evaluating one or more delivery parameters. Evaluation of one or more delivery parameters may occur in response to visualizing the pharmaceutical composition. Evaluation may occur before, during, or after the pharmaceutical composition is administered and/or visualized.

In some embodiments, the method comprising administering (e.g., by CED), visualizing (e.g., simultaneously) the pharmaceutical composition (e.g., comprising the radiotherapeutic) and evaluating one or more delivery parameters further comprises modifying one or more delivery parameters. In some embodiments, evaluating the one or more delivery parameters comprises maintaining (e.g., not changing) one or more previously existing delivery parameter (e.g., maintaining an infusion rate, concentration, dose, and/or volume). In some embodiments, modifying the one or more delivery parameter comprises modifying the one or more delivery parameters. In some embodiments, modifying the one or more delivery parameters comprises lowering a delivery parameter, elevating a delivery parameter, ceasing a delivery parameter or beginning a delivery parameter. In cases embodiments, the one or more delivery parameter comprises any one or more of the dose of the pharmaceutical composition, the dose of radiation, the volume of infusate, the infusion rate, the site(s) of administration, the administration intervals, the number of catheters, the rate of infusate per catheter, the interval for infusion (e.g., administration), the device delivering the pharmaceutical composition, the number of catheters (e.g., changing to one, two, three or four catheters), the rate per catheter, or any combination thereof. In some embodiments, one or more delivery parameters comprises one delivery parameter. In some embodiments, one or more delivery parameters comprises two, three, four, or five delivery parameters. The one or more delivery parameter may be any delivery parameter disclosed herein.

In some embodiments, administering (e.g., by CED) and visualizing (e.g., simultaneously) the pharmaceutical composition further comprises ensuring delivery of the pharmaceutical composition to the target (e.g., tumor). In some cases, ensuring delivery of the pharmaceutical composition to the target comprises delivering a minimally effective dose of the pharmaceutical composition to the target. In some cases, ensuring delivery of the pharmaceutical composition to the target comprises delivering of at least a portion (e.g., at least 10%, at least 20%, at least 30%, or at least 40%) of the pharmaceutical composition. In some cases, ensuring delivery comprises delivering at least about 50%, at least about 60%, at least about 70%, at least about 75%, at least about 80%, at least about 90%, at least about 95%, at least about 96%, at least about 97%, about least about 98%, or at least about 99% of the pharmaceutical composition.

In some embodiments, visualizing (e.g., imaging) occurs after administering (e.g., by CED) the pharmaceutical composition. Visualization post-administration may be useful for modifying one or more delivery parameters for subsequent administration(s) of the pharmaceutical composition. In some embodiments, visualizing occurs at 0 minutes, 1 minute, 2 minutes, 3 minutes, 4 minutes, 5 minutes, 10 minutes, 15 minutes, 20 minutes, 25 minutes, 30 minutes, 40 minutes, 45 minutes, 50 minutes, 1 hour, 1.25 hours, 1.5 hours, 2 hours, 4 hours, 5 hours, 10 hours, 24 hours, 48 hours, 72 hours, 4 days, 5 days, 1 week, 8 days, 2 weeks, or 4 weeks after administrating the pharmaceutical composition. In some embodiments, visualizing occurs before administering the pharmaceutical composition (e.g., comprising radiolabeled liposome (e.g., ¹⁸⁶Re)). In some embodiments, visualizing occurs at 1 minute, 2 minutes, 3 minutes, 4 minutes, 5 minutes, 10 minutes, 15 minutes, 20 minutes, 25 minutes, 30 minutes, 40 minutes, 45 minutes, 50 minutes, 1 hour, 1.25 hours, 1.5 hours, 2 hours, 4 hours, 5 hours, 10 hours, 24 hours, 48 hours, 72 hours, 4 days, 5 days, 6 days, 8 days, 1 week, 2 weeks, or 4 weeks before administrating the pharmaceutical composition. In some embodiments, visualizing occurs after beginning administration of the pharmaceutical composition. In some embodiments, visualizing occurs while administering the pharmaceutical composition.

In some embodiments, visualizing (e.g., imaging) comprises any of the imaging methods disclosed herein (e.g., SPECT, SPECT-CT, MRI, etc.).

In some embodiments, visualizing (e.g., the radiotherapeutic (radiolabeled liposome (e.g., ¹⁸⁶Re)) delivery) comprises imaging the whole body. In some embodiments, visualizing comprises imaging of a region (e.g., locoregion) of the body (e.g., the head). In some embodiments, visualizing comprises imaging the target (e.g., tumor). In some embodiments, visualizing comprises imaging of a primary tumor. In some embodiments, visualizing comprises imaging of a secondary tumor. In some embodiments, visualizing comprises imaging of a primary tumor and a secondary tumor. In some embodiments, visualizing comprises imaging of a tumor and the area surrounding the tumor (e.g., the region in which the tumor is located and/or within 6 inches of the tumor). In some embodiments, visualizing comprises imaging one or more organs (e.g., comprising a tumor and/or within the region of the tumor). In some embodiments, visualizing comprises imaging of a primary tumor.

In some embodiments, provided herein are methods of increasing survival in a subject having a cancer, comprising delivering greater than 100 Gy of radiation to the subject. In some embodiments, delivering greater than 100 Gy of radiation to the subject comprises delivering a pharmaceutical composition (e.g., ¹⁸⁶Re nanoliposomes). In some embodiments, provided herein are methods of increasing survival in a group of subjects having a cancer, comprising delivering greater than 100 Gy of radiation to one or more subjects in the group of subjects. In some embodiments, the method comprises delivering greater than 100 Gy to each of the one or more subjects in the group of subjects. In some embodiments, survival comprises overall survival. In some embodiments, the absorbed dose of radiation comprises the absorbed dose of β-rays. In some embodiments, are methods of increasing overall survival in a group of patients comprising glioblastoma (e.g., recurrent glioblastoma), comprising delivering greater than 100 Gy of radiation (e.g., β-rays) to the patient. In some embodiments, delivering greater than 100 Gy of radiation to the one or more subjects in the group comprises delivering a pharmaceutical composition (e.g., ¹⁸⁶Re nanoliposomes).

In some embodiments, a group of subjects having a cancer (e.g., glioblastoma) receiving greater than 100 Gy (e.g., absorbed dose) of radiation (e.g., β-rays) comprise an increased survival (e.g., overall survival) as compared to a second group of subjects comprising one or more subjects having the cancer (e.g., glioblastoma) receiving less than 100 Gy of radiation (e.g., β-rays). In some embodiments, the method comprises delivering less than 100 Gy of radiation to each of the one or more subjects in the second group of subjects. In some embodiments, absorbing the dose comprises administering the pharmaceutical composition disclosed herein (radiolabeled liposome (e.g., ¹⁸⁶Re)). In some embodiments, administering the pharmaceutical composition to the one or more subjects in the group of subjects results in an absorbed radiation dose of greater than 100 Gy.

In some embodiments, the methods provided herein increase overall survival. In some embodiments, overall survival comprises the number of days the subject lives after administration of the pharmaceutical composition (e.g., comprising radiolabeled liposome (e.g., ¹⁸⁶Re nanoliposome)). In some embodiments, the overall survival of subjects receiving greater than 100 Gy comprises about 100 days, about 150 days, about 200 days, about 250 days, about 300 days, about 350 days or about 400 days. In some embodiments, the overall survival of subjects receiving the pharmaceutical composition in amounts insufficient to result in greater than 100 Gy (e.g., achieving a dose less than 100 Gy) comprises about 50 days, about 100 days, about 125 days, or about 150 days. In some embodiments, the overall survival of subjects receiving greater than 100 Gy is greater than the overall survival of a second group of subjects receiving less than 100 Gy by about 20 days, about 30 days, about 45 days, about 60 days, about 70 days, about 80 days, about 90 days, about 100 days, about 110 days, about 120 days, or about 130 days.

### ALTERNATIVE EMBODIMENTS

Provided herein, in some embodiments, are radiotherapeutics (e.g., ¹⁸⁶Re nanoliposomes) for delivery of β-rays and gamma rays. In some embodiments, the radiotherapeutic is incorporated into a pharmaceutical composition.

Also provided herein are methods of a pharmaceutical composition (e.g., ¹⁸⁶Re nanoliposomes), comprising administering (e.g., by CED) the pharmaceutical composition (e.g., ¹⁸⁶Re nanoliposomes) in amounts sufficient to emit β-rays for the treatment of a cancer (e.g., glioblastoma) and gamma rays for the visualization of the of the pharmaceutical composition (e.g., ¹⁸⁶Re nanoliposomes), and visualizing (e.g. imaging) delivery of the pharmaceutical composition. In some embodiments, the method further comprises ensuring delivery of the pharmaceutical composition to the target (e.g., tumor). In some embodiments, the method further comprises modifying one or more delivery parameters. In some embodiments, modifying one or more delivery parameters occurs while administering the pharmaceutical composition. In some embodiments, modifying one or more delivery parameters occurs after administering the pharmaceutical composition.

Also provided herein are methods of delivery increments of β-rays to subjects (e.g., patients) comprising administering increments of a pharmaceutical composition (e.g., ¹⁸⁶Re nanoliposomes). In some embodiments, the subject received a previous dose of therapy. In some embodiments, the subject previously received a maximum dose of therapy. In some embodiments, the previous dose of therapy was a radiation therapy. In some embodiments, the subject received a previous dose of therapy for the treatment of a primary disease. In some embodiments, the subject received a previous dose of therapy for the treatment of a secondary disease.

Also provided herein are methods of increasing overall survival in a group of subjects (e.g., patients) with a cancer (e.g., glioblastoma), comprising delivering greater than 100 Gy of radiation (e.g., β-rays). In some embodiments, the group of subjects receiving greater than 100 Gy of radiation (e.g., β-rays) results in a greater overall survival than compared to a group of subjects receiving less than 100 Gy of radiation (e.g., β-rays).

Further provided herein are methods of delivering high (e.g, greater 100 Gy) absorbed radiation doses comprising delivering a pharmaceutical composition (e.g., ¹⁸⁶Re nanoliposomes). In some embodiments, the method of delivering high (e.g, greater 100 Gy) absorbed radiation doses comprises varying catheter number, flow rate (e.g., of infusate) and volume of infusate. In some embodiments, the method of delivering high (e.g, greater 100 Gy) absorbed radiation doses comprises varying catheter number. In some embodiments, the method of delivering high (e.g, greater 100 Gy) absorbed radiation doses comprises varying flow rate. In some embodiments, the method of delivering high (e.g, greater 100 Gy) absorbed radiation doses comprises varying the volume of infusate.

In an embodiment, up to 13.2 cc of radiation is delivered via the supratentorial region to a single hemisphere of the brain containing an existing tumor in the same hemisphere.

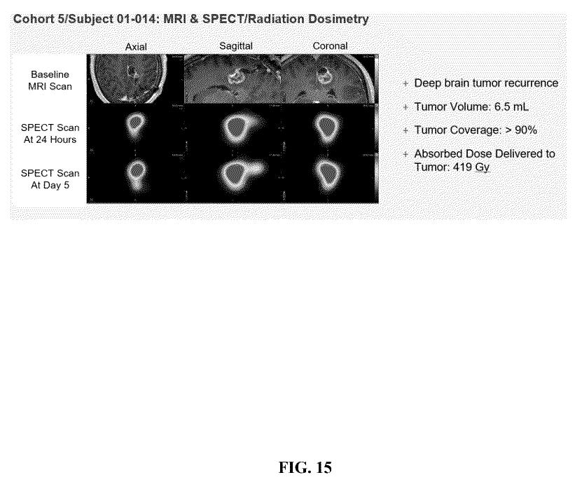

FIG. 15 shows baseline magnetic resonance images (MRIs) and single-photon emission computerized tomography (SPECT) images following treatment with 186Re nanoliposomes. MRI scans revealed an initial increase in size which peaked at Day 118, with some associated edema, pseudo-progression. They also revealed tumor shrinkage out to at least Day 362. The subject remained alive at 160 weeks after a single treatment.

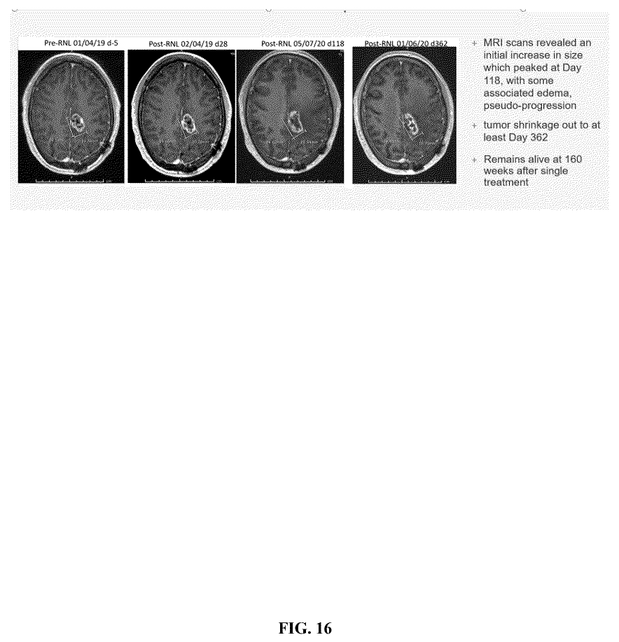

FIG. 16 shows MRI scans of tumor response observed to Day 362.

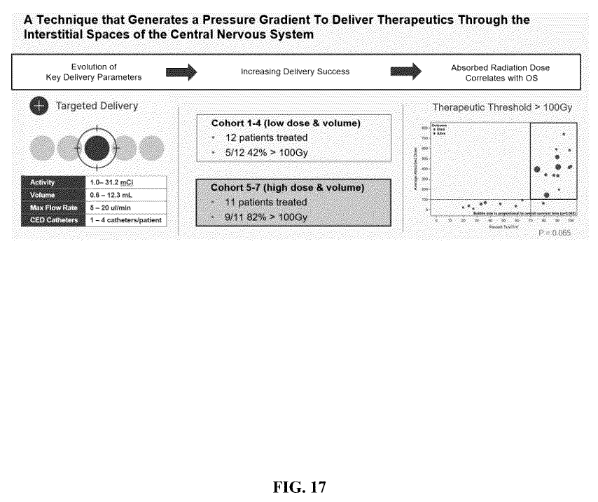

FIG. 17 shows a process of convection enhanced delivery to treat GBM.

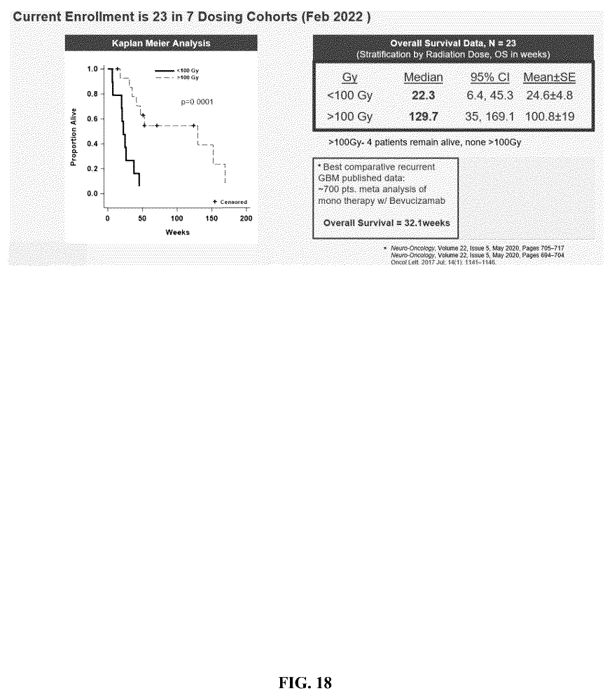

FIG. 18 shows efficacy and survival data for ReSPECT-GBM.

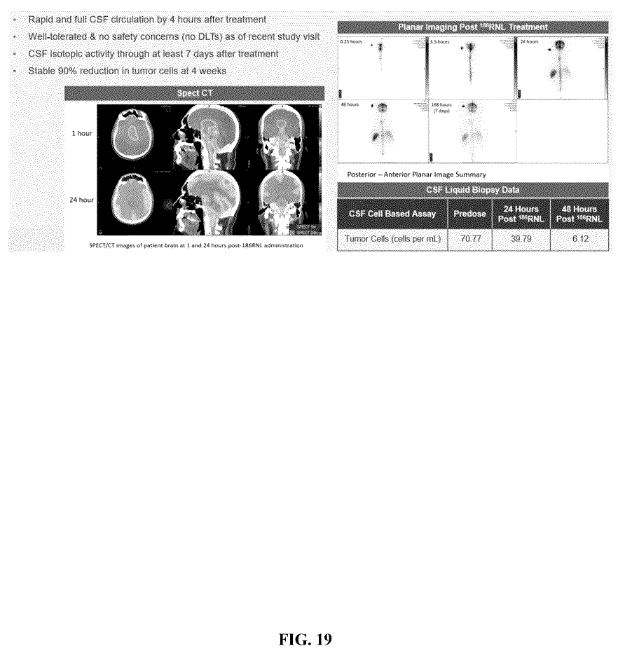

FIG. 19 shows SPECT CT, planar imaging and CSF Liquid Biopsy results for a patient post 186RNL treatment.

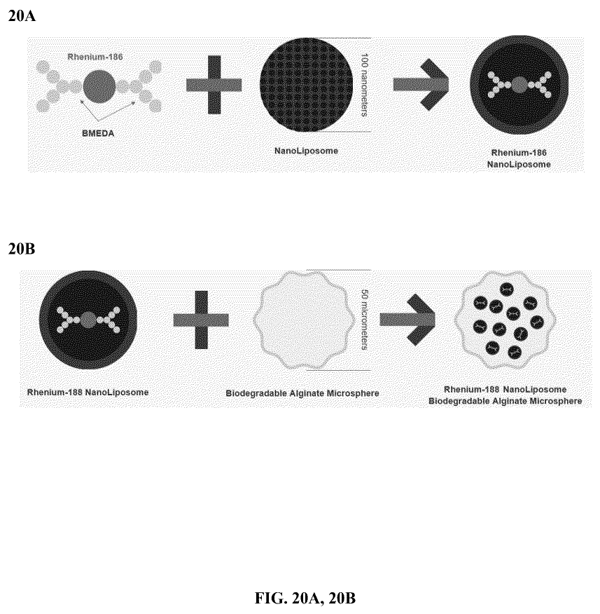

FIG. 20A shows a process or producing a Rhenium-186 NanoLiposome and FIG. 20B shows a process of producing a Rhenium 188 NanoLiposome Biodegradable Alginate Microsphere.

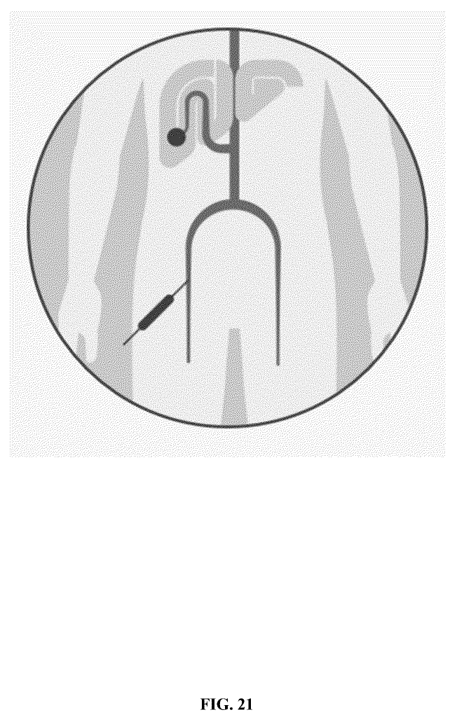

FIG. 21 shows an approach for a non-surgical locoregional treatment option for solid organ tumors. In an embodiment, a single intra-arterial injection of 188RNL-BAM in which biodegradable microspheres block the blood flow to the targeted solid organ tumors and simultaneously deliver a therapeutic payload of radiation. The Potential Advantages 188RNL-BAM may offer compared to the 2 radioembolization therapies currently available:
Biodegradable microspheres
Higher quality imaging
Work-up predictive of final clinical outcome
Shorter production time
Improved patient access
Higher margins
Better translate to other indications
   Liver cancer is the 6^{th} most common and 3^{rd} deadliest cancer. The treatments disclosed herein pursue new and relevant routes of administration and mechanisms of delivery and action. The treatments provide the opportunity to extend the life of patients with liver cancer through a safer, more targeted, convenient treatment approach.

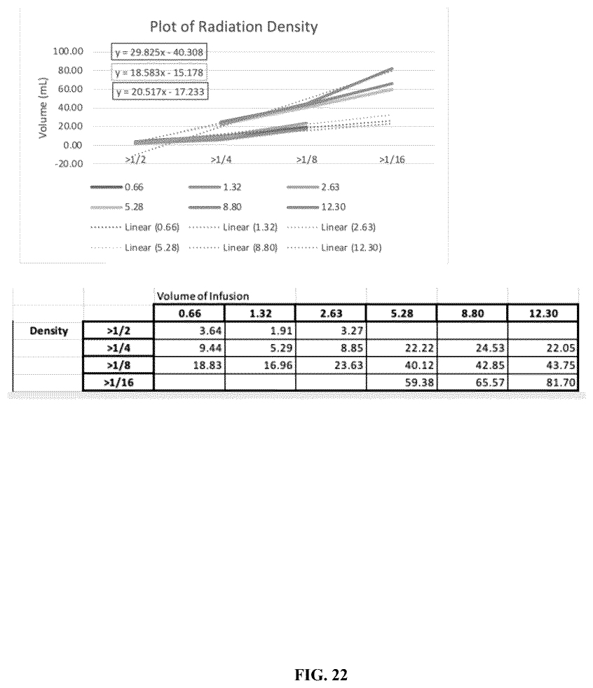

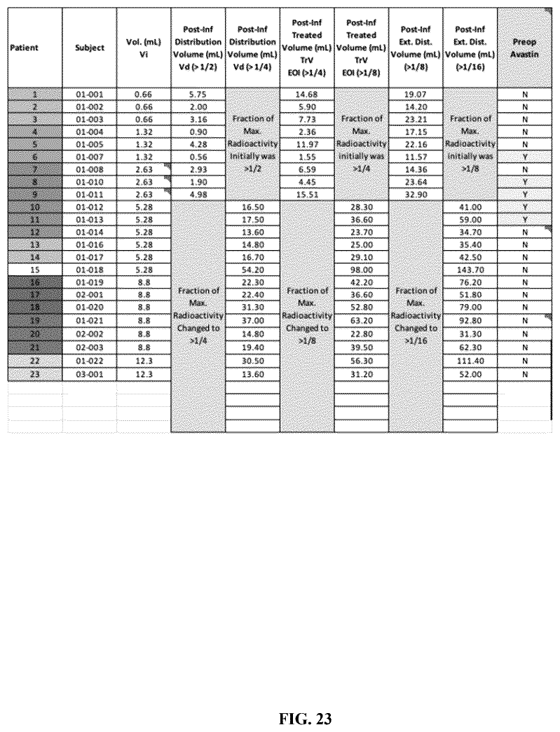

Regarding FIGS. 22 and 23, the figures indicate how much radiation volume is produced by the infused volume and correlations to the absorbed dose. In an embodiment, an infused volume of XX mL and YY mCi of radiation produces a volume of radiation of AA at (either <¼, <½, <⅛ or <1/1 of the average absorbed dose to the tumor.

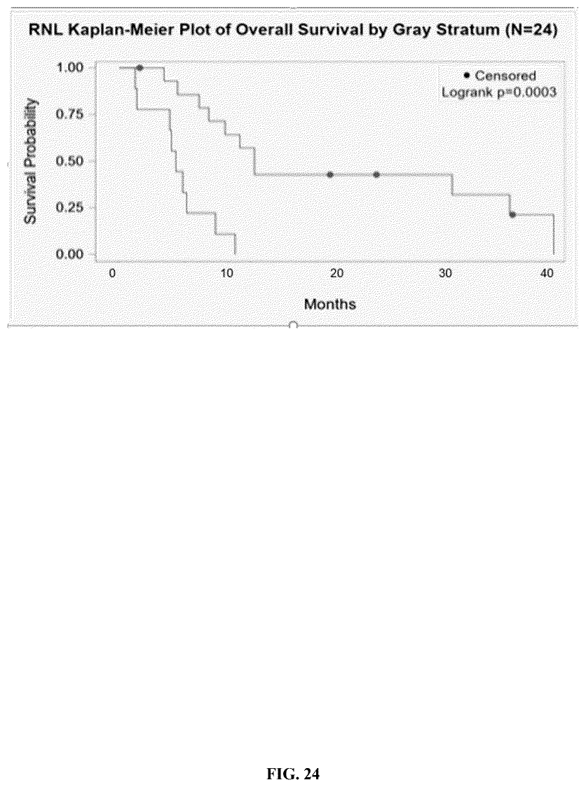

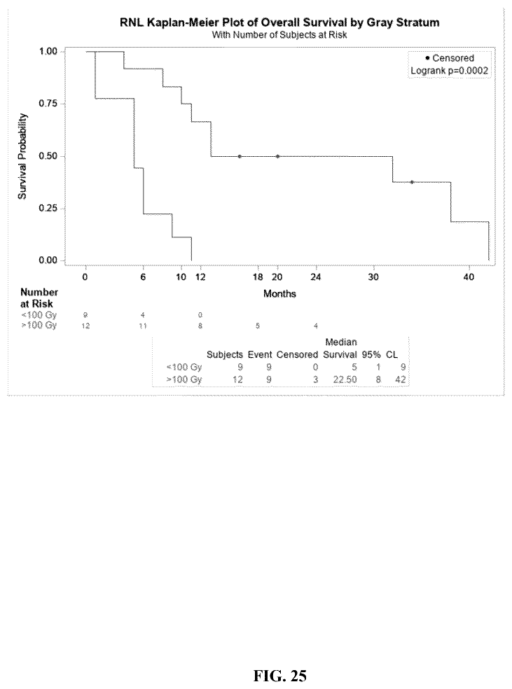

FIG. 24 shows a RNL Kaplan-Meier plot comparing the overall survival of patients receiving greater than 100 Gy as compared to patients receiving less than 100 Gy for up to 40 months. The plot compares patients receiving therapeutic (e.g., greater than 100 Gy) (red) vs. nontherapeutic radiation (e.g., less than 100 Gy) (blue). The group of patients receiving greater than 100 Gy had a greater overall survival as compared to the group of patients receiving less than 100 Gy. FIG. 25 shows a RNL Kaplan-Meier plot comparing the overall survival of patients receiving greater than 100 Gy as compared to patients receiving less than 100 Gy for 42 months.

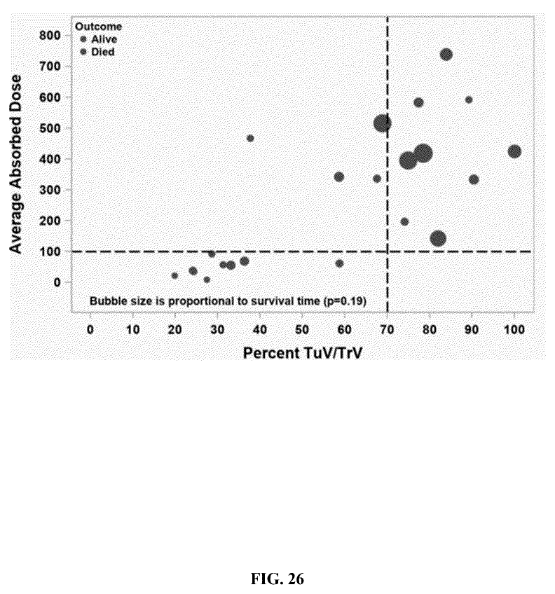

FIG. 26 shows a mortality plot with dose and tumor coverage. Bubble size is proportional to overall survival time. Alive is colored green and died is colored red. It appears that survival time increases with both absorbed dose and percent TuV/Trv.

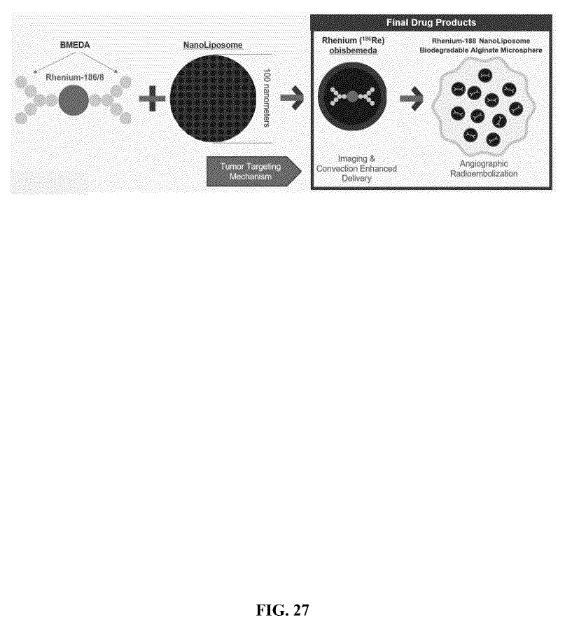

FIG. 27 shows a radiotherapeutic formulation of rhenium (186Re) obisbemeda and a rhenium-188 nanoliposome biodegradable alginate microsphere.

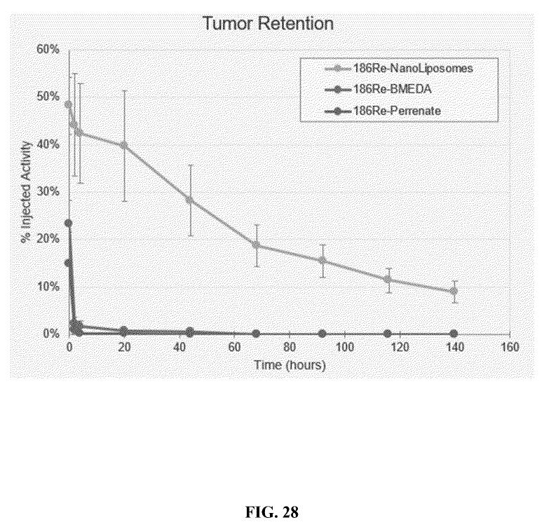

FIG. 28 shows a plot of tumor retention based on percent injected activity versus time (CED Intratumoral Administration in Head/Neck SCC Xenografts in Nude Rats).

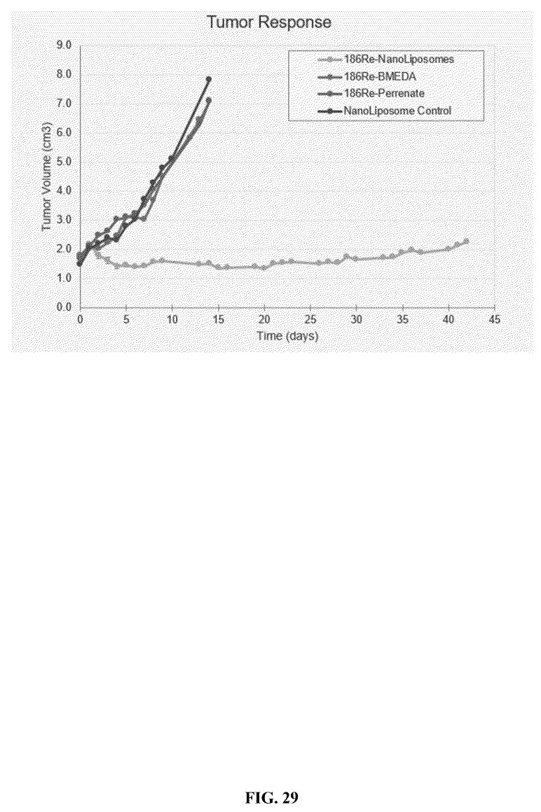

FIG. 29 shows a plot of tumor response based on tumor volume versus time (CED Intratumoral Administration in Head/Neck SCC Xenografts in Nude Rats).

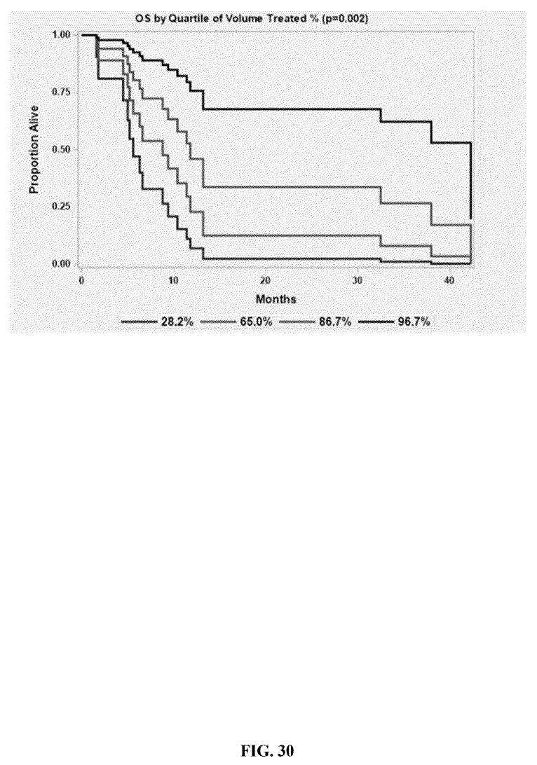

FIG. 30 shows percent volume tumor treated with proportion alive versus months. OS=overall survival.

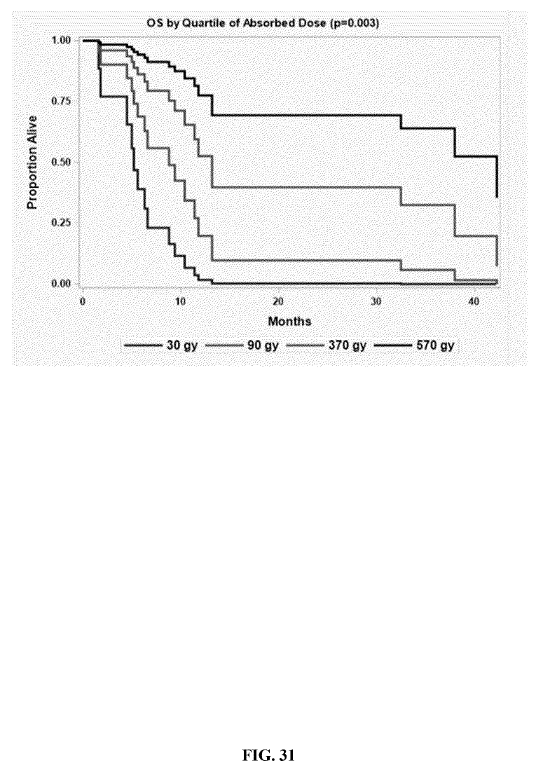

FIG. 31 shows average absorbed dose to tumor with proportion alive versus months.

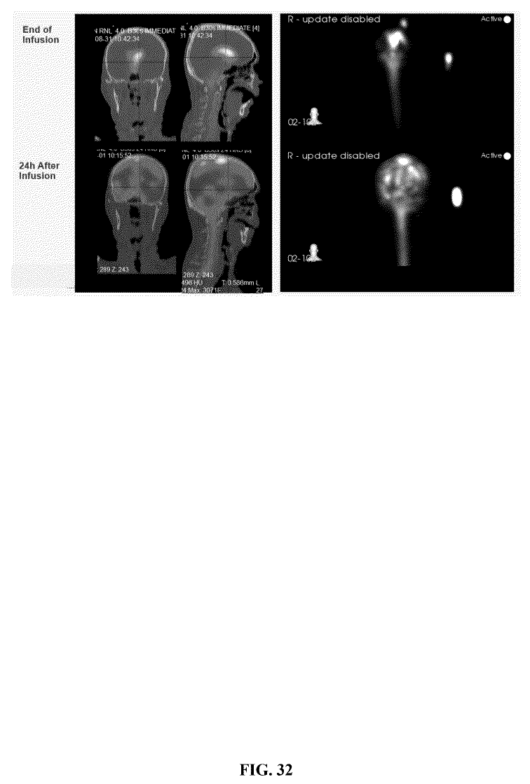

FIG. 32 shows leptomeningeal administration of rhenium (186Re) obisbemeda targeted to the CSF and rapid diffusion.

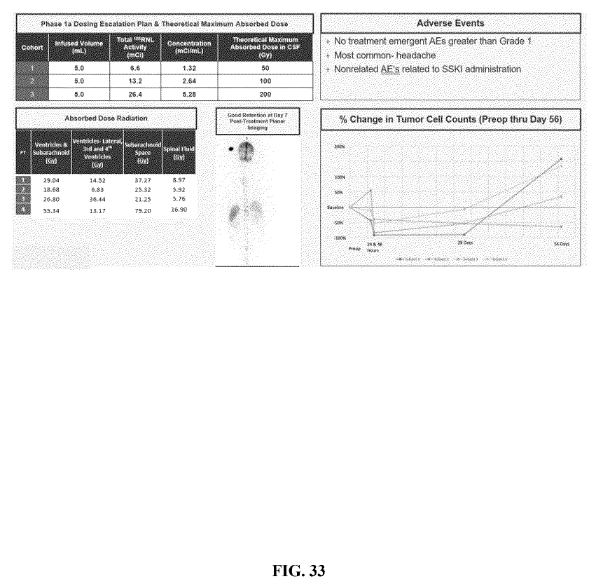

FIG. 33 shows updates on the ReSPECT-LM Clinical Trial.

### Exit Velocity

Exit velocity is based on the fluid volume that is expelled in a set time and the opening size through which the volume is expelled. Exit Velocity (in conjunction with Flow Rate and Catheter Tip Inner Diameter (ID)). The Exit Velocity=Volume rate of flow/Area (v=Q/A). The smaller the opening or resistance to exit, the high the exit velocity. The exit velocity affects the distribution of fluid that is being expelled. It would be expected that there is a greater distribution of fluid with a higher exit velocity.

TABLE 1

| Infusion | | | Exit Velocity | | Exit Velocity | | Exit Velocity |
|---|---|---|---|---|---|---|---|
| Flow | | Tip ID | (mm/min) | Tip ID | (mm/min) | Tip ID | (mm/min) |
| Rate | | Brainlab Smart Flow Flex | | Cleveland Multiport | | Renishaw Neuro Infuse | |
| 2 | ul/min | 0.021″ | 0.009 | .007″ | 0.081 | 0.014″ | 0.020 |
| 5 | ul/min | (0.533 mm) | 0.022 | (0.178 mm) | 0.201 | (0.35 mm) | 0.050 |
| 10 | ul/min | | 0.045 | | 0.403 | | 0.101 |
| 15 | ul/min | | 0.067 | | 0.604 | | 0.151 |
| 20 | ul/min | | 0.090 | | 0.806 | | 0.201 |

### EXAMPLE

$$Q = 20 ul / min = 0.02 ml / min = 0.02 {mm}^{3} / min$$

$$A = π {r}^{2} = 3.14 * 0.267 mm * 0.267 mm ( Smart Flow )$$

$$A = 0.223 {mm}^{2}$$

$$v = 0.02 {mm}^{3} / min / 0.223 {mm}^{2} = \text{.090} mm / min$$

### EXAMPLES

### Example 1: Phase 1 Clinical Trial

A single center, sequential cohort, open-label, volume- and dose-escalation Phase 1 clinical trial of the safety, tolerability, and distribution of ¹⁸⁶Re nanoliposomes administered via convection-enhanced delivery to patients with recurrent or progressive malignant glioma after standard surgical, radiation, and/or chemotherapy treatment was conducted. The study utilized a modified Fibonacci dose escalation and a standard 3+3 design.

Brainlab iPlan Flow software was used to plan SmartFlow catheter placement in the tumor volume while avoiding white matter tracts and cerebrospinal fluid spaces such as fissures, sulci, cisterns, ventricles, and resection cavities. Frameless image-guided catheter placement was achieved with Brainlab Varioguide Stereotactic system. ¹⁸⁶Re nanoliposomes were administered by convection-enhanced delivery utilizing 1 to 3 catheters at a maximum flow rate of up to 0.90 mL per hour per catheter (15 μL per minute per catheter).

Serial 1-minute dynamic planar imaging was performed during the time of the infusion. Single-photon emission computerized tomography (SPECT)/computerized tomography (CT) imaging and serial whole-body planar imaging scans were performed immediately following, and at 1, 3, 5, and 8 days after ¹⁸⁶Re nanoliposomes infusion to assess the radiation absorbed dose to the tumor and other organs during the treatment. Serial blood samples and serial 24-hour urine collections were also counted for activity. Dosimetry was performed using region of interest data and OLINDA dose calculation software.

Fifteen patients were treated, of which fourteen had recurrent glioblastoma and 47% failed treatment with bevacizumab. The infused dose was progressively increased from 1.0 mCi to 13.4 mCi and the volume of infusate was increased from 0.66 mL to 5.28 mL using 1 to 3 convection-enhanced delivery catheters. The therapy was well tolerated and no dose-limiting toxicity was observed despite markedly higher absorbed doses than external beam radiation therapy (EBRT) in patients with prior treatment. The study design and overall survival of each patient is summarized in Table 2 below.

TABLE 2
Phase 1 Clinical Trial Design

| | | Volume | | | | | | | |
|---|---|---|---|---|---|---|---|---|---|
| | | of | | | Maximum | Prior | | Overall | |
| | Dose | Infusate | Conc. | | Flow Rate | Treatment | Bevacizumab | Survival | |
| Patient | (mCi) | [Vi] (mL) | (mCi/mL) | Catheters | (μL/min) | Cycles | Failure | (months) | Alive |
| 1 | 1.0 | 0.66 | 1.5 | 1 | 5 | 1 | N | 30.0 | N |
| 2 | 1.0 | 0.66 | 1.5 | 1 | 5 | 1 | N | 33.0 | N |
| 3 | 1.0 | 0.66 | 1.5 | 1 | 5 | 2 | N | 8.6 | N |
| 4 | 2.0 | 1.32 | 1.5 | 1 | 5 | 3 | Y | 1.6 | N |
| 5 | 2.0 | 1.32 | 1.5 | 1 | 5 | 1 | N | 10.8 | N |
| 6 | 2.0 | 1.32 | 1.5 | 1 | 5 | 2 | Y | 5.7 | N |
| 7 | 4.0 | 2.64 | 1.5 | 1 | 5 | 2 | Y | 4.1 | N |
| 8 | 4.0 | 2.64 | 1.5 | 1 | 5 | 2 | Y | 5.7 | N |
| 9 | 4.0 | 2.64 | 1.5 | 1 | 5 | 2 | Y | 4.1 | N |
| 10 | 8.0 | 5.28 | 1.5 | 1 | 10 | 3 | Y | 4.8 | N |
| 11 | 8.0 | 5.28 | 1.5 | 2 | 10 | 2 | Y | 4.9 | N |
| 12 | 8.0 | 5.28 | 1.5 | 2 | 10 | 1 | N | 22.0 | Y |
| 13 | 13.4 | 5.28 | 2.5 | 2 | 10 | 3 | N | 11.0 | Y |
| 14 | 13.4 | 5.28 | 2.5 | 3 | 15 | 2 | N | 3.6 | Y |
| 15 | 13.4 | 5.28 | 2.5 | 3 | 15 | 2 | N | 3.3 | Y |

The median survival duration in patients that had failed bevacizumab treatment (n=7) was 4.8 months. The median survival duration in bevacizumab-naïve patients (n=8) was 10.9 months, with 4 patients still alive at the conclusion of the study.

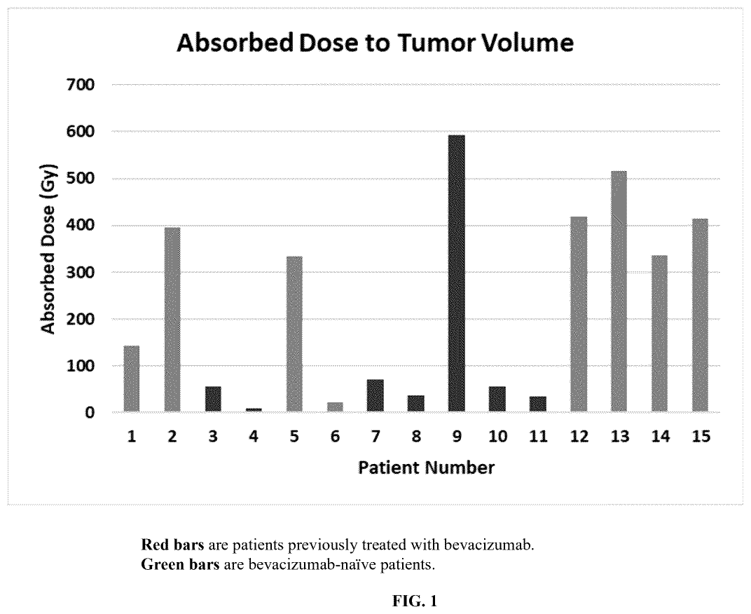

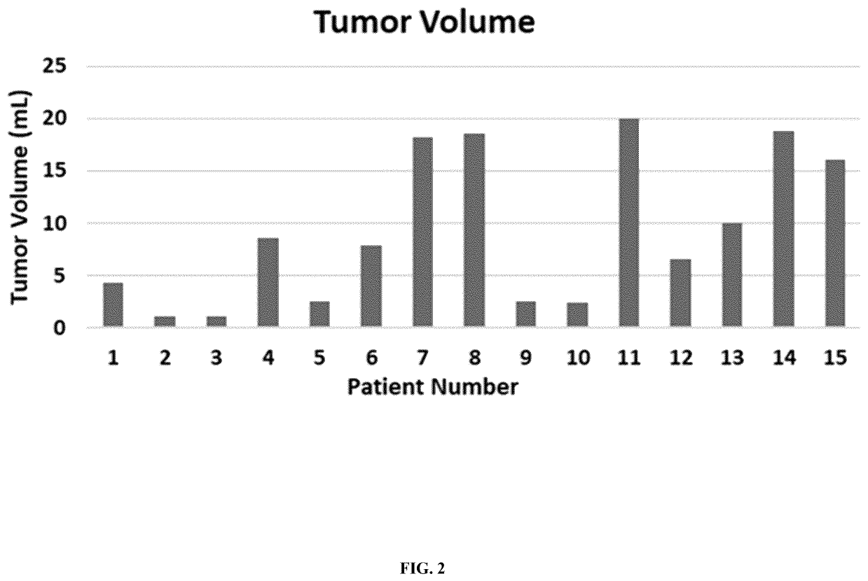

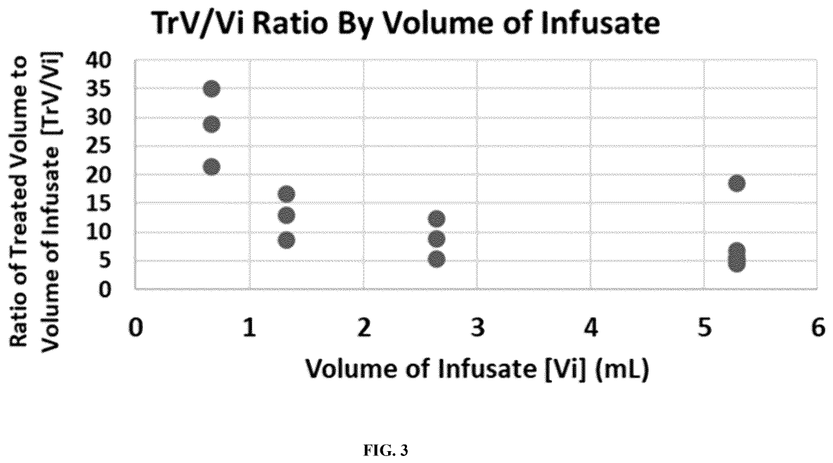

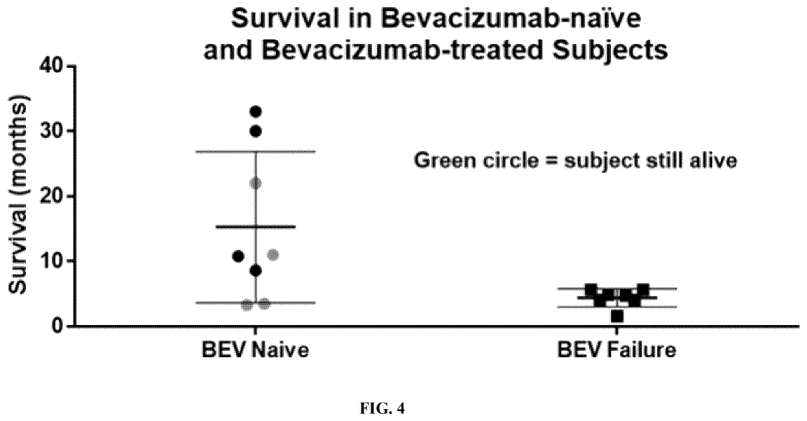

The absorbed dose of ¹⁸⁶Re nanoliposomes to the tumor volume for patients previously treated with bevacizumab and bevacizumab-naïve patients is summarized in FIG. 1. The tumor volume for each patient is summarized in FIG. 2. The ratio of treated volume to volume of infusate versus the volume of infusate is summarized in FIG. 3. The difference in survival between patients previously treated with bevacizumab and bevacizumab-naïve patients is summarized in FIG. 4.

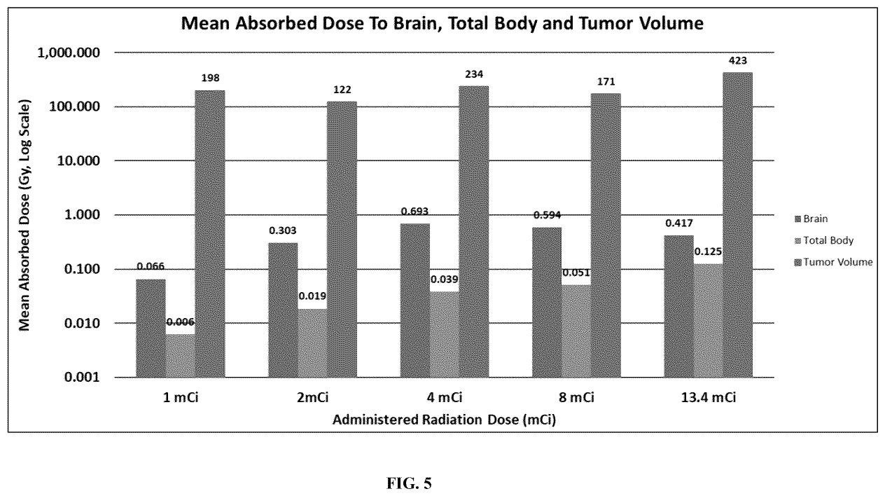

The mean absorbed dose to brain, total body, and tumor volume for the various dose levels of ¹⁸⁶Re nanoliposomes are summarized in FIG. 5. The median absorbed dose to the tumor volume across all patients (n=15) was 143 Gy (range 9-593 Gy). The median absorbed dose in patients who had failed bevacizumab (n=7) was 38 Gy (range 9-593 Gy), while the median absorbed dose in bevacizumab-naïve patients (n=8) was 367 Gy (range 56-517 Gy). The maximum absorbed dose to the tumor volume was 593 Gy. The mean retention of the administered dose at 24 hours was 64% (Cl 50-78). The total body absorbed dose was less than or equal to 0.70 Gy. The mean absorbed dose to the organs was less than or equal to 0.70 Gy. The ratio of the mean tumor volume absorbed dose to the mean total body absorbed dose in the last two cohorts was greater than or equal to 3,000.

The adverse events with the highest incidence were fatigue (40%), diarrhea (27%), gait abnormality (27%), headache (27%), hemiparesis (27%), lightheadedness (20%), scalp discomfort (20%), seizure (20%), and cerebral edema (20%). Neither the incidence nor severity of adverse events appeared to increase with increasing doses of ¹⁸⁶Re nanoliposomes. Four serious adverse events were reported: seizure in 3 patients and cerebral edema in 1 patient. However, none of these serious adverse events were considered causally related to ¹⁸⁶Re nanoliposomes treatment. Most adverse events were considered causally unrelated to ¹⁸⁶Re nanoliposomes treatment except for scalp discomfort, which was considered related to the surgical procedure.

### Example 2: Case Study

The patient was a 51-year-old white female who presented with neurologic symptoms and possible seizure. Magnetic resonance imaging (MRI) of the brain showed a right frontal mass. Craniotomy revealed grade 2 diffuse astrocytoma, IDH wild-type, MGMT unmethylated (cIMPACT-NOW grade IV). She was treated with combined radiation and temozolomide followed by three cycles of maintenance temozolomide. The follow-up MRI was consistent with recurrent disease. The patient entered a Phase 0 trial of Sacituzumab with repeat right frontal craniotomy 9 months after initial presentation of recurrent glioblastoma and underwent the first dose in cycle 1 (C1D1) of Sacituzumab one month later. Disease progression was noted again 16 months after initial presentation. Less than 4 weeks later, the patient was treated with ¹⁸⁶Re nanoliposomes (13.4 mCi, 5.28 mL, 3 catheters).

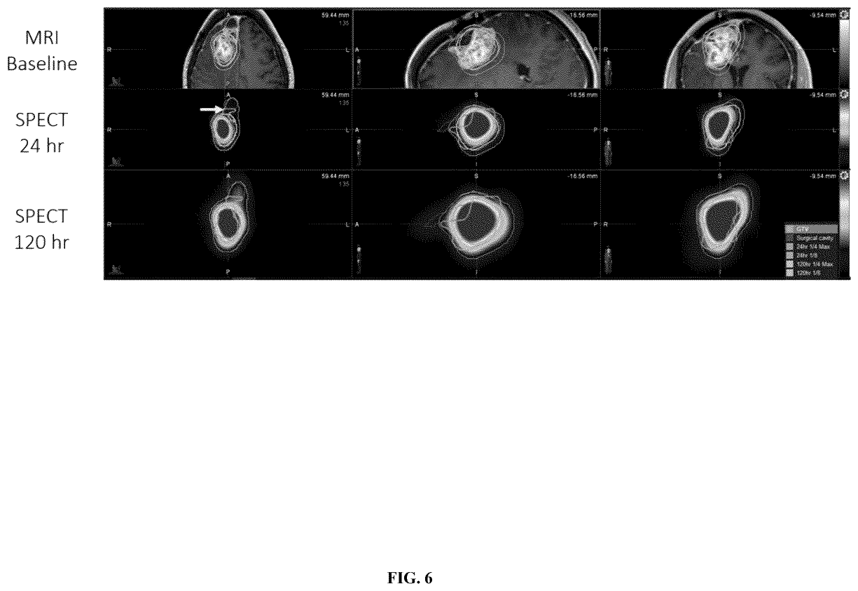

The MRI and SPECT images at baseline, 24 hours, and 120 hours post-treatment are shown in FIG. 6. The tumor volume was 18.8 mL and tumor coverage was 87%. The absorbed dose delivered to the tumor was 336 Gy and to the distribution volume (Vd) was 245 Gy. At 24 hours, the anterior portion of the tumor (shown with a yellow arrow in FIG. 6) was outside of the distribution volume (shown with a gold line in FIG. 6). By day 5 post-treatment, the entire tumor was within the distribution volume (shown with a light purple line in FIG. 6).

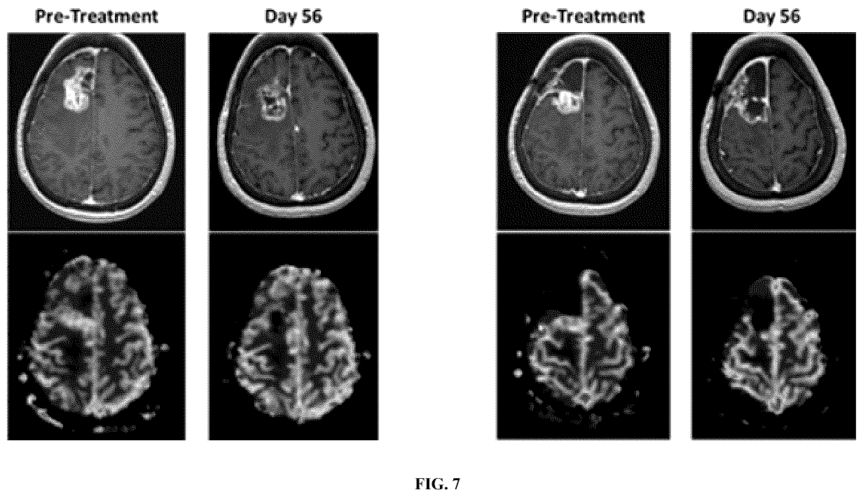

Magnetic resonance images (MRIs) and perfusion scan images at baseline and 56 days following treatment with ¹⁸⁶Re nanoliposomes are shown in FIG. 7. The MRI on day 56 showed partial response without evidence of edema. The perfusion scans showed significant decrease in previously enhancing tumor region.

As evidenced by the above examples, intratumoral ¹⁸⁶Re nanoliposomes can deliver up to twenty times the absorbed dose of radiation administered by external beam radiation therapy without significant toxicity. Intratumoral administration of ¹⁸⁶Re nanoliposomes by convection-enhanced delivery at doses up to 13.4 mCi in 5.28 mL of infusate is safe and well-tolerated. Prior administration of bevacizumab appears to negatively impact convection of ¹⁸⁶Re nanoliposomes.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 3: Image-Guided Rhenium-186 NanoLiposome (¹⁸⁶RNL) Brachytherapy in the Treatment of Recurrent Glioblastoma: Technique, Image Analysis, Dosimetry, and Monitoring

¹⁸⁶Rhenium-lipid nanoparticles (¹⁸⁶Re nanoliposomes comprising ¹⁸⁶RNL) has been introduced in the focused brachytherapy of cancers via image-guided infusion to the tumor interstitial space; firstly, in the treatment of recurrent glioblastoma of brain (GBM). Rhenium-186 (¹⁸⁶Re) is a radionuclide (half-life: 89.24 hours) emitting therapeutic beta-radiation particles (path range: 1.8 mm). One of every 10 emissions is associated with a 137 KeV gamma ray allowing imaging of in vivo drug distribution, retention, and for radiation absorbed dose distribution calculation and therapy evaluation. Herein, we report our Phase I clinical Trial study with the focus on technique used, image analysis, radiation dosimetry, and therapy monitoring.

While external beam radiation therapy (EBRT) remains a central component of the management of gliomas, it is limited by tolerance of the surrounding normal brain tissue. We hypothesized that Rhenium-186 NanoLiposome (¹⁸⁶RNL) (e.g., ¹⁸⁶Re nanoliposomes) focused brachytherapy may permit the selective delivery of high specific activity beta-emitting radiation with excellent retention in the tumor and that the emitted gamma rays will permit real time image-guided delivery and monitoring. Herein, we report the technique used, imaging of the nanoliposome distribution and retention, image analysis, radiation dosimetry, and therapy monitoring.

A Phase 1 clinical trial of ¹⁸⁶RNL administered by convection enhanced delivery (CED) through a Brainlab flexible subacute catheter to treat recurrent glioblastoma was conducted. BrainLab iPlan Flow Software and Varioguide system were used for treatment planning and catheter placement. The retention and distribution of ¹⁸⁶RNL (e.g., ¹¹⁶Re nanoliposomes) within the tumor and whole body were obtained by planar and SPECT/CT imaging from mid-infusion to 8 days following radiation source administration. Locoregional and whole body drug retention were analyzed from whole body planar images. SPECT/CT and follow-up MRI images were co-registered to planning MRI images for 3D drug distribution and therapy evaluation. Radiation absorbed doses to local volumes and whole body organs were calculated.

Using MIM MRT quantitative SPECT image reconstruction and 3D dose calculation, 3D radiation absorbed dose distribution of ¹⁸⁶RNL therapy in 8 days has been calculated, and evaluated with tumor therapy response.

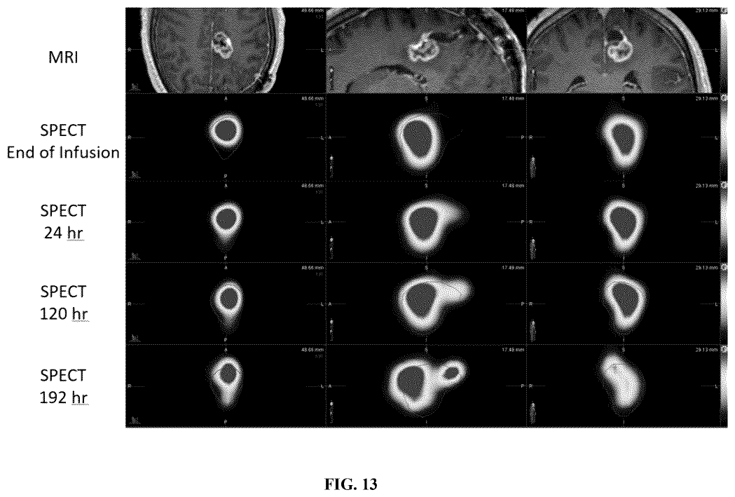

As shown in FIG. 13, ¹⁸⁶RNL (e.g., ¹¹⁶Re nanoliposomes) delivered by CED to patients with recurrent glioblastoma may result in predictable distribution and stable retention of nanoparticle radiation source in the targeted tissues, providing days of sustained, localized radiation treatment to the tumor. FIG. 13 shows the distribution and retention of ¹⁸⁶RNL radiation source in 8 days after infusion. ¹⁸⁶RNL radiation source had sustained retention and stable locoregional distribution at tumor to be treated in the brain, which provided the continuous radiation therapy effect in over 8 days. The subject from whom these images were obtained comprised a tumor volume of 6.5 mL and tumor coverage was greater than 90%. The absorbed dose delivered to the tumor was 419 Gy.

The use of up to four catheters effectively enhances locoregional drug distribution and tumor volume coverage as well as the speed of infusion improving patient convenience. The mean locoregional retention in the brain volume at the end of infusion was 85.9%±17.1% ID (n=18) and at 8 days post-infusion was 46.6%±16.7% ID (n=17). Following dose escalation, the mean radiation absorbed dose in the two most recent cohorts to the tumor volume was 354.7±144.0 Gy, to the whole brain was 1.32±1.06 Gy, and to the whole body was 0.16±0.04 Gy (n=6).

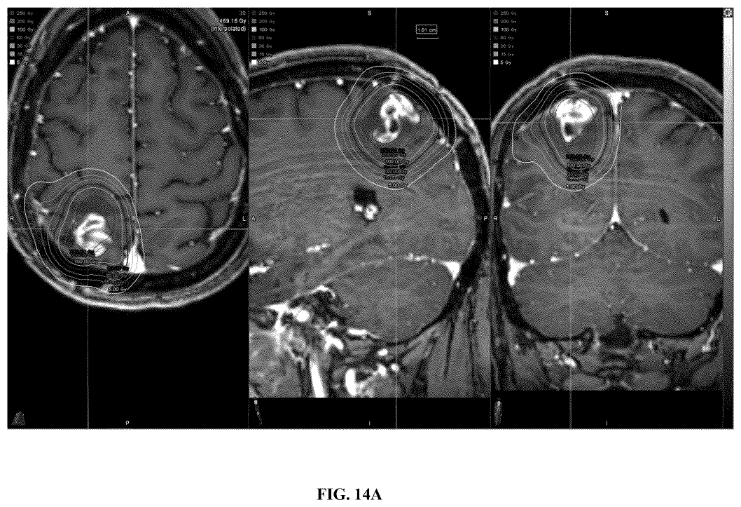

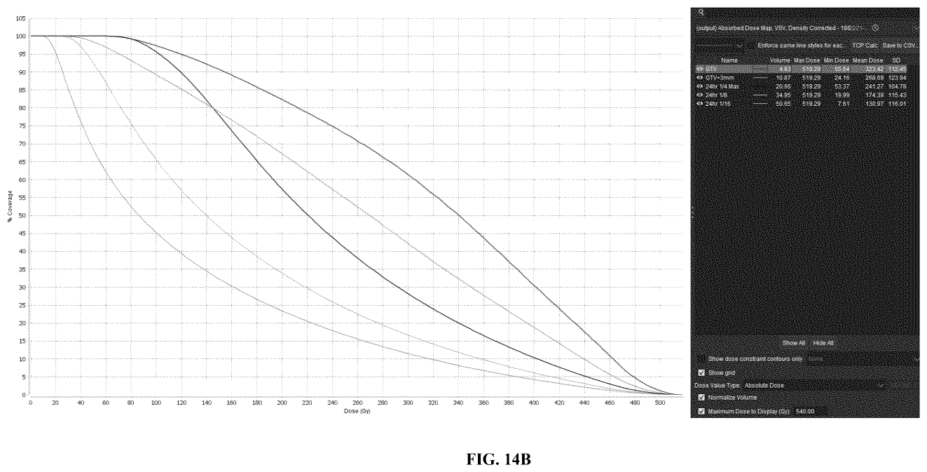

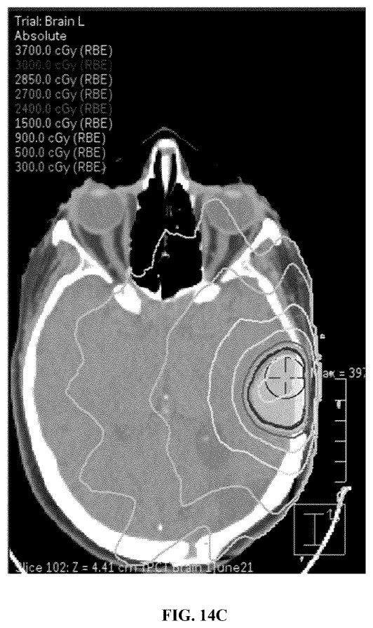

FIG. 14A shows distribution of ¹⁸⁶RNL (e.g., ¹⁸⁶Re nanoliposomes) brachytherapy, and FIG. 14B shows a dose volume histogram of ¹⁸⁶RNL (e.g., ¹⁸⁶Re nanoliposomes) brachytherapy. As shown, the maximum dose in the treated volume was >500 Gy, the mean dose to GTV was 323 Gy, and minimum dose to GTV was 55.6 Gy in 8 days. An important feature of the treatment technique may be the highly focused treatment to the volume with radiation source delivery. The 3D dose distribution has shown a very sharp dose gradient; the 5 Gy isodose line has been shown (white arrow). For comparison, FIG. 14C illustrates the isodose distribution of an SBRT case in a tumor and the surrounding normal tissue. In the SBRT case, a 30-Gy prescribed dose to the radiotherapy planning target volume (PTV) was delivered. The white line shows the 5-Gy isodose line.

The focused brachytherapy treatment allows for a high radiation dose treatment for tumor eradication, while with low toxicities to un-involved tissue. With the treatment technique, the radiation absorbed dose in the treated volume were a few hundred Gy or higher, while radiation absorbed dose beyond the distribution volume of radiation source dropped rapidly to a minimal level; for reference see the white arrow in FIG. 14A, which shows the 5-Gy isodose line.

Based on the results of this study, ¹⁸⁶RNL (e.g., ¹⁸⁶Re nanoliposomes) brachytherapy may provide a high radiation absorbed dose to the tumor with minimal brain and whole body radiation exposure. The therapy from this technique may also provide a sustained radiation to the target from high to low radiation dose rates for over 8 days, which can be beneficial in radiobiology on tumor control.

Results of this study demonstrate that, as compared with sealed source brachytherapy, ¹⁸⁶RNL (e.g., ¹⁸⁶Re nanoliposomes) brachytherapy may be advantageous in minimal invasiveness, planning, convenience of delivery, and that this therapy is bioresorbable. Furthermore, the image monitoring capability may provide a predictive tool to evaluate therapy delivery and treatment effectiveness, and the development of 3D dose distribution calculation may provide a convenient mechanism for dose and therapy effect evaluation, and for the application of additional therapy for better tumor control.

### Example 4: Safety and Feasibility of Rhenium-186 NanoLiposome (¹⁸⁶RNL) in Recurrent Glioma

EBRT, a central component of the management of primary brain tumors, it is limited by tolerance of the surrounding normal brain tissue. Rhenium-186 NanoLiposome (¹⁸⁶RNL) permits the delivery of beta-emitting radiation of high specific activity with excellent retention in the tumor.

A Phase 1 dose-escalation study of ¹⁸⁶RNL in recurrent glioma utilized a standard 3+3 design was to determine the maximum tolerated dose of ¹⁸⁶RNL ¹⁸⁶RNL was administered by convection enhanced delivery (CED). Infusion was followed under whole body planar imaging and SPECT/CT. Repeat SPECT/CT imaging was performed immediately following administration, and at 1, 3, 5, and 8 days after ¹⁸⁶RNL infusion to obtain dosimetry and distribution. Subjects were followed until disease progression by RANO criteria. Eighteen subjects were treated across 6 cohorts, each cohort consisting of 3 subjects. Table 3 shows information related to the dosing of each cohort, such as the amount of radioactivity delivered, the infusate volume, and concentration of the infusate. The mean tumor volume was 9.4 mL (range 1.1-23.4). The infused dose ranged from 1.0 mCi to 22.3 mCi and the volume of infusate ranged from 0.66 mL to 8.80 mL. 1-4 CED catheters were used for delivery. The maximum catheter flow rate was 15 μl/min. The mean absorbed dose to the tumor volume was 239 Gy (CI 141-337; range 9-593), to normal brain was 0.72 Gy (CI 0.34-1.09; range 0.005-2.73), and to total body was 0.07 Gy (CI 0.04-0.10; range 0.001-0.23). The mean absorbed dose to the tumor volume when the percent tumor volume in the treatment volume was 75% or greater (n=10) was 392 Gy (CI 306-478; range 143-593). Scalp discomfort and tenderness related to the surgical procedure occurred in 3 subjects. The therapy was well tolerated, no dose-limiting toxicity has been observed, and no treatment-related serious adverse events have occurred despite markedly higher absorbed doses typically delivered by EBRT in patients with prior treatment. Responses were observed supporting the clinical activity.

¹⁸⁶RNL administered by CED to patients with recurrent glioma resulted in a higher absorbed dose of radiation to the tumor compared to EBRT, without significant toxicity. In view of the results, ¹⁸⁶RNL may be useful for the treatment of recurrent glioma. Specifically, a dose of 22.3 mCi in 8.8 mL of infusate may be useful for the treatment of recurrent glioma.

TABLE 3
Dose characteristics.

| | | Infusate | | Average |
|---|---|---|---|---|
| | Activity | Volume | Concentration | Absorbed |
| Cohort | (mCi) | (mL) | (mCi/mL) | Dose (Gy) |
| 1 | 1.0 | 0.66 | 1.5 | 198 |
| 2 | 2.0 | 1.32 | 1.5 | 122 |
| 3 | 4.0 | 2.64 | 1.5 | 234 |
| 4 | 8.0 | 5.28 | 1.5 | 171 |
| 5 | 13.4 | 5.28 | 2.5 | 423 |
| 6 | 22.3 | 8.80 | 2.5 | 287 |

### Example 5: Overall Survival of Rhenium-186 Nanoliposomes in Recurrent Glioblastoma (GBM)

Glioblastoma (GBM) is the most common and aggressive primary malignant brain tumor in adults. The standard treatment for GBM over the last decade has been surgery followed by concomitant chemoradiotherapy with temozolomide, with radiation being a major contributor to survival in this regimen. As most recurrences may occur within two centimeters of the resection margin, loco-regional therapies that bypass the blood brain barrier are attractive potential alternatives. External beam radiation therapy (EBRT) is a central component of primary brain tumor management but may be limited by tolerance of surrounding normal brain and skeletal tissue. Rhenium-186 (¹⁸⁶Re) is a potent source of electrons with short path length, low dose rate and high radiation density. Specifically, ¹⁸⁶Re may be a β-ray-emitting therapeutic radionuclide with a 90-hour half-life, 1.8-mm radiation path range, and high β/γ-energy ratio suitable for cancer brachytherapy. Additionally, ¹⁸⁶Re may have an energy of gamma ray sufficient to allow imaging of the in vivo radiopharmaceutical distribution with standard SPECT/CT.

Rhenium-186 nanoliposomes may permit the delivery of beta radiation of high specific activity with excellent retention in the tumor. Therapeutic radionuclides may require a carrier to ensure they are sequestered within the tumor and slowly redistributed. Liposomal nanoparticles (nanoliposomes) may provide a means of encapsulating radionuclides and assisting in sustained intratumoral accumulation while also convecting within the tumor. We have successfully developed a method of loading ¹⁸⁶Re into nanoliposomes with high efficiency and specific activity. This process may result in a markedly higher level of specific activity than has been previously described and has the potential to provide a markedly higher delivered therapeutic radiation doses with decreased toxicity. Preclinically, liposomal encapsulated ¹⁸⁶RNL (e.g., ¹⁸⁶Re nanoliposomes) delivered via convection enhanced delivery (CED) may achieve a very high doses of targeted radiation and a wide therapeutic index. We report the results of the first in man phase 1 trial of ¹⁸⁶RNL (e.g., ¹⁸⁶Re nanoliposomes) in recurrent glioma (ReSPECT).

The study is a multi-center, sequential cohort, open-label, volume and dose-escalation Phase 1 clinical trial of the safety, tolerability, and distribution of RNL (e.g., ¹⁸⁶Re nanoliposomes) given by convection enhanced delivery (CED) to patients with recurrent or progressive malignant glioma after standard surgical, radiation, and/or chemotherapy treatment. The study uses a modified Fibonacci dose escalation and a standard 3+3 design.

Brainlab iPlan Flow software was used to plan SmartFlow catheter placement in the tumor volume while avoiding white matter tracts and CSF spaces (fissures, sulci, cisterns, ventricles and resection cavities). Frameless image-guided catheter placement was achieved with Brainlab Varioguide Stereotactic system. A single administration of RNL is delivered by CED utilizing 1-4 catheters at a maximum flow rate of up to 20 μL/min/catheter.

Serial 1-minute dynamic planar imaging was performed during the time of the infusion. SPECT/CT imaging and serial whole-body planar imaging scans were performed immediately following, and at 1, 3, 5, and 8 days after RNL (e.g., ¹⁸⁶Re nanoliposomes) infusion to assess the radiation absorbed dose to the tumor and other organs during the treatment. Serial blood samples and serial 24-hour urine collections were also counted for activity. Dosimetry was performed using region of interest data and OLINDA dose calculation software. The dose escalation scheme for each cohort is shown in Table 4.

TABLE 4
Dose escalation scheme for each cohort.

| | Infusate | Total RNL | | Average | |
|---|---|---|---|---|---|
| | Volume | Activity | Concentration | Absorbed | |
| Cohort | (mL) | (mCi) | (mCi/mL) | Dose (Gy) | Status |
| 1 | 0.66 | 1.0 | 1.5 | 198 | Enrolling |
| 2 | 1.32 | 2.0 | 1.5 | 122 | cohort 7 |
| 3 | 2.64 | 4.0 | 1.5 | 234 | (n = 22 |
| 4 | 5.28 | 8.0 | 1.5 | 171 | subjects) |
| 5 | 5.28 | 13.4 | 2.5 | 423 | |
| 6 | 8.80 | 22.3 | 2.5 | 287 | |
| 6* | 8.80 | 22.3 | 2.5 | 584 | |
| 7 | 12.3 | 31.2 | 2.5 | TBD | |

*Cohort 6* utilized same volume and dose as cohort 6 but with increase in maximum flow rate to 20 microliters/minute.

Twenty-two patients across 7 dosing cohorts were treated from 2015 to 2021. Fourteen were male and eight were female. Average tumor size was 8.3 mL (range 0.9-22.8 mL). The average number of prior treatments (Txs) was 1.7 (range 1-3 Txs). Five patients received prior Bevacizumab. The pathologic grade was Grade IV glioma in 20 patients and Grade III in 2 patients. IDH mutational status was wild type in 18 patients and mutated, in 2 patients (2 patients had no status) and MGMT status was methylated in 4 patients and unmethylated in 12 patients (6 patients had no status).

Twenty-two patients across 7 dosing cohorts received a range of 1.0-31.2 mCi in a volume of 0.6-12.3 mL. The maximum CED administration rate range was 5-20 μl/min and 1-4 catheters were used per patient. Mean absorbed radiation dose to the tumor was 273 Gy (8.9-740 Gy) while exposure outside the brain was negligible. The tumor (TuV) in the treated volume (TrV) or percent TuV/TrV, was 70.9% (19.8%-100%) and correlated to the average absorbed radiation dose to the tumor. In 5 patients receiving prior bevucizamab therapy, the average absorbed dose to TuV was 149.2 Gy and the percent TuV/TrV was 47.9%. However, in 17 patients not receiving prior bevacizamab average Absorbed dose to TuV patients was 302 Gy and the percent TuV/TrV was 77.7%. Further enrollment of bevucizamab patients was stopped based on the poor convection at these convection inputs.

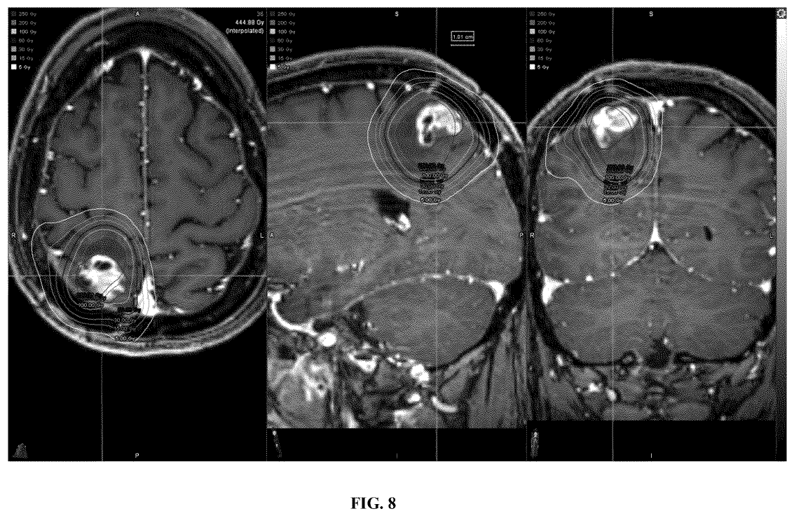

FIG. 8 shows a 3D view of the extent of radiation delivered (measured in absorbed dose). The image was taken 8 days posttreatment with ¹⁸⁶RNL (e.g., ¹⁸⁶Re nanoliposomes).

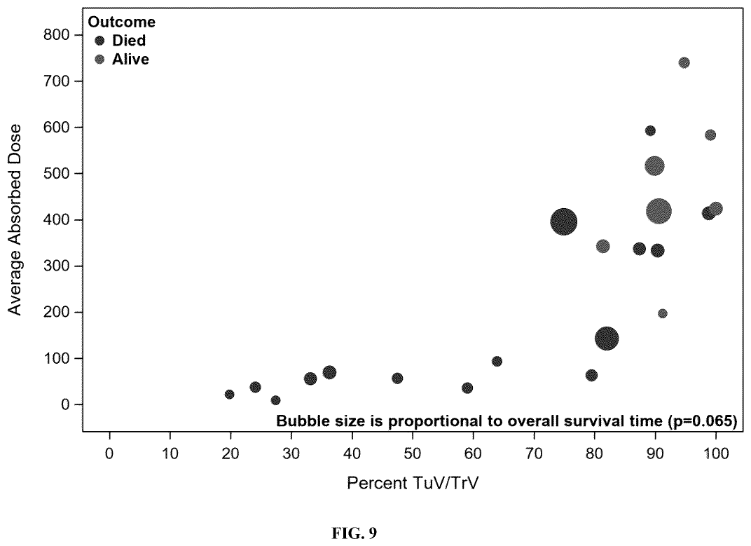

FIG. 9 shows a chart demonstrating the overall survival in terms of Average Absorbed Dose and Percent TuV/TrV. The analysis was based on a proportional hazards model of overall survival in days in terms of average absorbed dose, percent TuV/Trv, and the interaction term, suggesting that survival time increases with both absorbed dose and percent TuV/Trv (p=0.065) based on a sample size N=22.

In the 22 subjects with recurrent Glioblastoma (GBM) receiving a single administration of ¹⁸⁶RNL (e.g., ¹⁸⁶Re nanoliposomes), ¹⁸⁶RNL may be safe and well-tolerated. Specifically, there were no Adverse Events (AEs) with outcome of death, or discontinuations due to AEs. The majority of AEs reported were mild or moderate (Grade 1 or 2) in intensity and non-serious. The Adverse Events (AEs) with the highest incidence were: Fatigue (50.0%), muscular weakness and headache (33.3% each), and gait disturbance (27.8%). Most AEs were considered causally unrelated to ¹⁸⁶RNL except one case of scalp discomfort, which was considered related to the surgical procedure, and one case of cerebral edema. AEs with Grade 3 were leukocytosis, hyperglycemia, muscular weakness, seizure, brain edema, avascular necrosis of the shoulder (worsening), vasogenic cerebral edema and pneumonia; all these events were considered unrelated to ¹⁸⁶RNL by the Principal Investigator with the exception of brain edema for one subject, which was considered possibly related to ¹⁸⁶RNL.

Serious Adverse Events were reported for two subjects in cohort 2 (seizure and vasogenic cerebral edema), one subject each in cohort 4 and cohort 5 (both seizure), and two subjects in cohort 6 [pneumonia, avascular necrosis of the shoulder (worsening) and cerebral edema]. All these events were considered unrelated to ¹⁸⁶RNL (e.g., ¹⁸⁶Re nanoliposomes) by the Investigator with the exception of cerebral edema for one subject, which was considered possibly related to ¹⁸⁶RNL and/or tapering of oral corticosteroids and none led to study discontinuation.

There were no meaningful differences or patterns in the incidence of related TEAEs reported across individual treatment groups (cohorts). Neither the incidence nor severity of AEs appeared to increase with increasing doses of ¹⁸⁶RNL (e.g., ¹⁸⁶Re nanoliposomes).

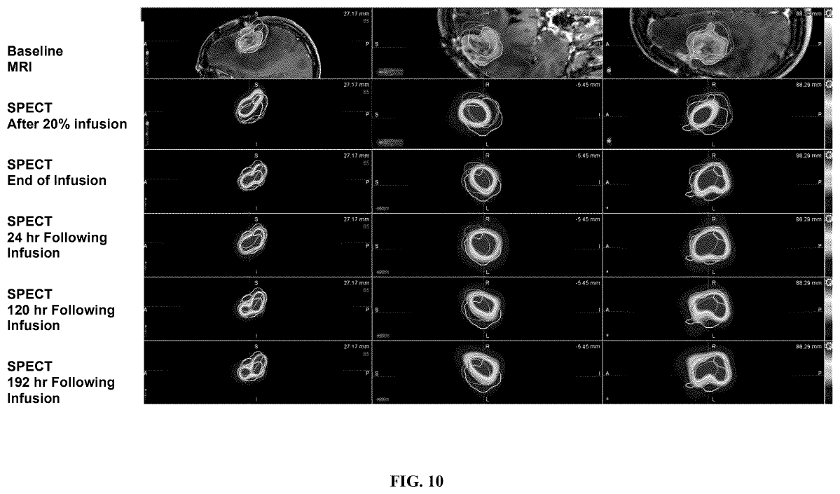

FIG. 10 shows an image comprising a baseline MRIs, and SPECT images after 20% infusion, at the end of infusion, 24 hours following infusion, 120 hours following infusion, and 192 hours following infusion.

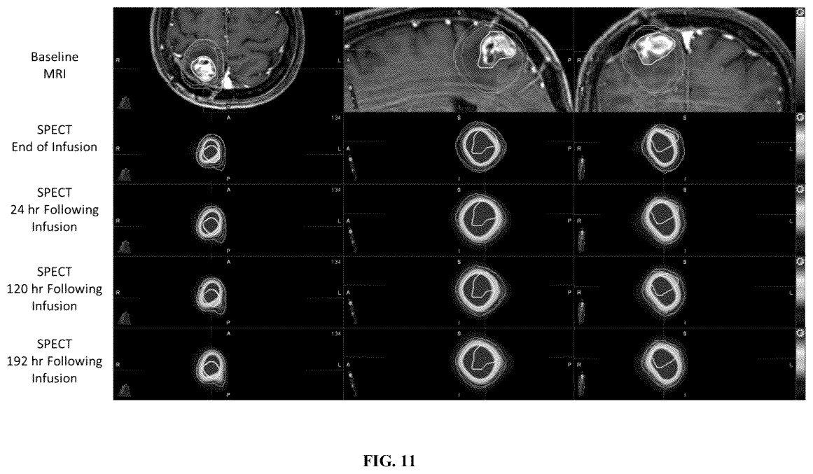

FIG. 11 shows an image comprising a baseline MRIs, and SPECT images at the end of infusion, 24 hours following infusion, 120 hours following infusion, and 192 hours following infusion.

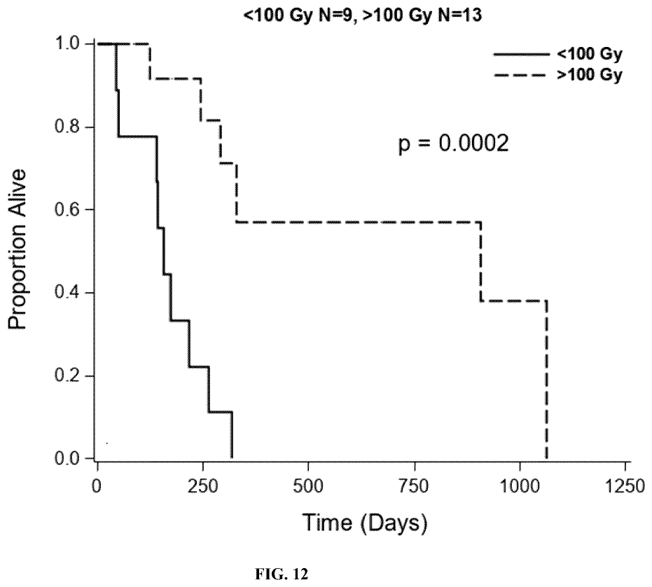

FIG. 12 shows a Kaplan Meier curve comparing patients receiving therapeutic (e.g., greater than 100 Gy) vs. nontherapeutic radiation (e.g., less than 100 Gy). The curve shows a statistically significant difference between the group (p=0.0002). As demonstrated, the group of patients receiving greater than 100 Gy had a greater overall survival as compared to the group of patients receiving less than 100 Gy. A statistically significant overall survival benefit was observed in patients achieving adequacy in absorbed radiation dose (>100 Gy) vs. those that do not. Additionally, adequacy in absorbed radiation dose (>100 Gy) can be achieved in 80% of patients treated in cohorts 5-7.

Based on the results of the study, intra-tumoral convection enhanced delivery of ¹⁸⁶RNL into the brain may precisely deliver up to twenty times the absorbed dose of radiation that can be administered by EBRT. Further, single administration of ¹⁸⁶RNL may be safe with no dose limiting toxicities observed, and SPECT/CT may accurately and reliably visualize the location and residual radioactivity level of the RNL as it decays. Lastly, this study demonstrated that increasing drug volume and radiation dose given in later dosing cohorts correlated with an improvement is overall survival.

### Example 6: a Study of Rhenium-186 NanoLiposome (¹⁸⁶RNL) Delivered by Convection Enhanced Delivery for Recurrent, Refractory, or Progressive Ependymoma and High-Grade Glioma (HGG) and Newly Diagnosed Diffuse Intrinsic Pontine Glioma (DIPG)

Ependymoma, HGG, and DIPG are often difficult to treat, frequently aggressive, and often carry a poor prognosis. While radiation therapy remains a mainstay of management, it is limited by tolerance of the surrounding brain tissue. Rhenium-186 NanoLiposome (¹⁸⁶RNL) (e.g., ¹⁸⁶Re nanoliposomes) permits the selective delivery of beta-emitting radiation of high specific activity with excellent retention in the tumor.

A two-part, Phase 1 dose-finding study followed by an expansion cohort to explore efficacy in pediatric patients based on data from a phase 1 trial in adults will be conducted. Part 1 enrolled up to 18 subjects to determine the maximum feasible dose of ¹⁸⁶RNL (e.g., ¹¹⁶Re nanoliposomes) administered by convection enhanced delivery (CED). Tumor diameter will be limited to 4 cm and a volume of 34 mL. The dose limiting toxicity period (DLT) is 28 days post infusion. Part 2 will independently evaluate ¹⁸⁶RNL (e.g., ¹¹⁶Re nanoliposomes) in 3 different cohorts: A: up to 12 subjects with a diagnosis of recurrent, refractory, or progressive ependymoma; B: up to 12 subjects with a diagnosis of recurrent, refractory, or progressive HGG; C: up to 15 subjects with newly diagnosed DIPG. The primary endpoint is overall response rate (ORR) by Radiographic Assessment in Pediatric Neuro-Oncology (RAPNO) criteria. Secondary endpoints are progression-free survival at 24 months (PFS-24) and overall survival at 24 months (OS-24) in Cohort A and PFS-12 and OS-12 in Cohorts B and C.

In a Phase 1 trial of adults with recurrent glioblastoma (NCT01906385), the mean dose to the tumor when coverage was 75% or greater (n=10) was 392 Gy (CI 306-478). Therapy was well tolerated, no dose-limiting toxicity was observed, and no serious adverse events were observed (n=18).

The delivery of ¹⁸⁶RNL (e.g., ¹⁸⁶Re nanoliposomes) administered by CED is well tolerated in adults with recurrent HGG with significantly greater doses of radiotherapy than with standard modalities. Further studies will be conducted in adult and pediatric subjects. ¹⁸⁶RNL administered by CED may be useful in treating HGG (e.g., recurrent HGG).

### Example 7: Maximum Tolerated Dose, Safety, and Efficacy of Intraventricular Rhenium-186 Nanoliposome (¹⁸⁶RNL) for Leptomeningeal Metastases

Leptomeningeal metastases (LM) are a rare but typically fatal complication of advanced cancer that affects the fluid-lined structures of the central nervous system and are diagnosed in approximately 5 percent of patients with metastatic cancer. With survival measured in weeks to months, novel approaches are needed that can both improve quality and quantity of life. Rhenium-186 nanoliposome (¹⁸⁶RNL) permits the selective delivery of beta-emitting radiation of high specific activity directly to the tumor. In a Phase 1 trial in adults with recurrent glioblastoma (NCT01906385), the mean absorbed dose to the tumor when coverage was 75% or greater (n=10) was 392 Gy (CI 306-478). Therapy was well tolerated with one possible treatment-related serious adverse event, cerebral edema, that resolved after steroid treatment.

This is a two-part, Phase 1 dose-finding study followed by an expansion cohort to explore efficacy. Part 1 will enroll up to 21 subjects to characterize the safety and tolerability of a single dose of ¹⁸⁶RNL administered intraventricularly via an Ommaya reservoir and to identify a maximum tolerated dose (MTD)/maximum feasible dose (MFD) for future studies. The dose limiting toxicity period is 28 days post infusion. Part 2 will independently evaluate ¹⁸⁶RNL in 2 different cohorts: Cohort A: up to 20 subjects with a diagnosis of LM from primary breast cancer; Cohort B: up to 20 subjects with a diagnosis of LM from primary non-small cell lung cancer. The primary endpoint is to estimate the anti-tumor activity of ¹⁸⁶RNL as a single agent. Secondary endpoints are to characterize the pharmacokinetic and dosimetry profile of a single dose of ¹⁸⁶RNL, determine the overall response rate (ORR) based on CSF and radiographic findings, and to describe the survival distribution.

### Example 8: Preclinical Safety and Activity of Intraventricular Rhenium-186 Nanoliposome (¹⁸⁶RNL) for Leptomeningeal Metastases

LM is a clinical complication that may occur when cancer cells invade the leptomeninges and cerebrospinal fluid of patients with malignant tumors. Once diagnosed, limited treatment options exist, and survival is poor. Rhenium-186 Nanoliposome (¹⁸⁶RNL) is a liposomal encapsulated beta emitter with a short path length of 1.8 mm, thereby allowing high specific activity brachytherapy with limited exposure to surrounding tissues. Therefore, ¹⁸⁶RNL may be useful in treating LM.

To establish the maximum tolerated dose (MTD) of ¹⁸⁶RNL by intraventricular (IT) injection, eight cohorts of Wistar rats (n=3 each) were injected IT with increasing activity of ¹⁸⁶RNL at doses of 0 (control), 0.480, 0.800, 1.000, 1.150, and 1.340 mCi. Toxicity was assessed by daily food and water intake, daily weights, and observing for neurological deficits. To assess efficacy, C6-Luc glioma cells were injected IT at 15 days post inoculation the animals were treated with 0.69 mCi of ¹⁸⁶RNL. Absorbed doses were assessed with gamma camera imaging at 0 h, 24 h, and 48 h post-treatment. Tumor growth was assessed by luciferase bioluminescence.

No evidence of adverse ¹⁸⁶RNL-related effects was observed in rats through 3 months following administration of up to 1.34 mCi with an absorbed dose of up to 1075 Gy. Hence, the MTD exceeded the doses evaluated in this study. A significant difference in survival between the control and treatment groups (n=8 each) was observed at 2 weeks post treatment, with 50% survival in the control group and 100% survival in the treatment group (p=0.0087). The only significant histologic finding among treated rats was thickening of the leptomeninges overlying the median eminence suggesting a mild reactive meningeal hypertrophy. This evidence demonstrates that intraventricular delivery of ¹⁸⁶RNL may be well tolerated and may improve animal survival at 2 weeks in a rat model of LM. This data demonstrates that ¹⁸⁶RNL may be useful for treating LM.

### Example 9: Maximum Tolerated Dose of ¹⁸⁶RNL

Nanoliposomal BMEDA-chelated-¹⁸⁶Rhenium (¹⁸⁶RNL) permits the delivery of beta-emitting radiation of high specific activity with excellent retention in the tumor. A phase 1 dose-escalation study of ¹⁸⁶RNL in recurrent glioma utilizing a standard 3+3 design was undertaken to determine the maximum tolerated dose of ¹⁸⁶RNL following stereotactic biopsy. ¹⁸⁶RNL is administered with the BrainLab Flexible Catheter by convection with placement guided using iPlan Flow and the Varioguide system. Infusion is followed under whole body planar imaging and SPECT/CT. Repeat SPECT/CT imaging is performed immediately following, and at 1, 3, 5, and 8 days after ¹⁸⁶RNL infusion to obtain dosimetry and distribution.

Thirteen patients were treated, 12 were recurrent glioblastoma, and 54% failed treatment with bevacizumab. The infused dose was progressively increased from 1.0 mCi to 13.4 mCi and the volume of infusate from 1.0 mL to 5.28 mL using 1-2 CED catheters. The mean absorbed dose to the distribution volume was 175 Gy (CI 97-254). The maximum absorbed dose to the tumor volume was 593 Gy. The mean retention of the administered dose at 24 hours was 61.4% (CI 45.4-77.5). The therapy has been well tolerated and no dose-limiting toxicity has been observed with no treatment related adverse effects despite markedly higher absorbed doses than EBRT in patients with prior treatment. The plan is to increase the dose to 22.3 mCi and the infusate volume to 8.8 mL. Based on the results of this study, intratumoral ¹⁸⁶RNL may deliver up to twenty times the absorbed dose of radiation administered by EBRT without significant toxicity.

### Example 10: An Open-Label Phase I Clinical Study that Will Administer a Single Dose of ¹⁸⁶RNL Via Intraventricular Catheter for Treatment of Leptomeningeal Metastases (LM)

This is a Phase I clinical study evaluates a single dose of ¹⁸⁶RNL (radionuclide clinical study drug) (e.g., ¹⁸⁶Re nanoliposomes in a pharmaceutical composition) administered through an intraventricular catheter (Ommaya reservoir) in participants with Leptomeningeal Metastases (LM). The clinical study treatment consists of a single administered 5 cc dose of ¹⁸⁶RNL per participant. The clinical study will include the evaluation of three separate dose levels. Three to six participants may be treated at each dose. The maximum number of participants to be enrolled in the study is 18. The clinical study treatment will be administered, following a CSF flow study, on an outpatient basis by the clinical study physician. Participants will be followed for up to 12 months after the clinical study drug is administered.

The study will comprise dose escalation for cohorts 1-3. Each participant will receive a single 5 cc administration of ¹⁸⁶RNL. At each dose level, a minimum of three to a maximum of six participants will be enrolled. If no dose limiting toxicity is observed in the initial three participants, then the next higher dose level cohort will open for enrollment. The dose escalation scheme will follow a modified Fibonacci dose escalation scheme as shown: Cohort 1 (6.6 mCi), Cohort 2 (13.2 mCi), Cohort 3 (26.4 mCi).

Outcome measures will comprise primary outcome measures and secondary outcome measures. Primary outcome measures will comprise: a.) incidence and severity of adverse events (AE) and serious adverse events (SAE) over a 12 month time frame (safety will be evaluated by the incidence of AEs and SAEs graded according CTCAE version 5.0), and b.) Incidence of dose-limiting toxicities (DLT) over a 12 month time frame (the Maximum Tolerated Dose (MTD) will be evaluated by testing increasing doses for cohorts 1 to 3 with 3 to 6 participants in each cohort. MTD reflects the highest dose of drug that did not cause a Dose-Limiting Toxicity (DLT) in >33% of participants).

Secondary outcome measures will comprise: a.) determination of the overall response rate (ORR) over a 12 month time frame (the overall response rate (ORR) defined as the proportion of all evaluable participants achieving a response as the best overall response at the time of progression will be determined); b.) determination of the duration of response (DoR) over a 12 month time frame (he duration of response (DoR) defined as the time from first response to LM progression will be determined); c.) determination of progression free survival (PFS) (progression free survival (PFS) defined as the time from first treatment to date of LM progression or death from any cause will be determined); and d.) determination of overall survival (OS) (the overall survival (OS) define as the time from first treatment to date of death will be determined).

Participant eligibility criteria includes the inclusion criteria and exclusion criteria. Inclusion criteria comprises the following: at least 18 years of age at time of screening; ability to understand the purposes and risks of the study and has signed a written informed consent document approved by the site-specific IRB; subject has proven and documented LM that meets the requirements for the study: EANO-ESMO Clinical Practice Guidelines Type 1 and 2 (with the exception of 2D) LM of any primary type; Karnofsky performance status of 60 to 100; a.) acceptable liver function: bilirubin≤1.5 times upper limit of normal, b.) AST (SGOT) and ALT (SGPT)≤5.0 times upper limit of normal, and c.) acceptable renal function with serum creatinine≤2 times upper limit of normal; acceptable hematologic status (without hematologic support): a). ANC≥1000 cells μL, b.) platelet count≥75,000/μL, c.) hemoglobin≥9.0 g/dL; all women of childbearing potential must have a negative serum pregnancy test at screening; male and female subjects must agree to use effective means of contraception (for example, surgical sterilization or the use of barrier contraception with either a condom or diaphragm in conjunction with spermicidal gel or an IUD) with their partner from entry into the study through 6 months after the last dose.

Exclusion criteria comprises the following: the subject has not recovered to National Cancer Institute (NCI) Common Terminology Criteria for Adverse Events (CTCAE v5.0 Grade≤1 from AEs (except alopecia, anemia and lymphopenia) due to antineoplastic agents, investigational drugs, or other medications that were administered prior to study; obstructive or symptomatic communicating hydrocephalus; ventriculo-peritoneal or ventriculo-atrial shunts without programable valves or contraindications to placement of Ommaya reservoir; females of childbearing potential who are pregnant, breast feeding, or may possibly be pregnant without a negative serum pregnancy test; serious intercurrent illness, such as progressive systemic (extra leptomeningeal) disease, clinically significant cardiac arrhythmias, uncontrolled systemic infection, symptomatic congestive heart failure or unstable angina pectoris within 3 months prior study drug, myocardial infarction, stroke, transient ischemic attack within 6 months, seizure disorder with any seizure occurring within 14 days prior to consenting or encephalopathy; active severe non hematologic organ toxicity such as renal, cardiac, hepatic, pulmonary, or gastrointestinal systemic toxicity grade 3 or above; significant coagulation abnormalities such as inherited bleeding diathesis or acquired coagulopathy with unacceptable risks of bleeding; craniospinal irradiation (for intraparenchymal or dural metastasis) or intrathecal cytotoxic anti-cancer therapy less than 3 weeks prior to first dose of ¹⁸⁶RNL; myelopathy following spinal irradiation greater than 3 weeks prior to the first dose of ¹⁸⁶RNL; systemic chemotherapeutic agents with CNS penetration (such as temozolomide, carmustine, lomustine, capecitabine, carboplatin, vinorelbine, bevacizumab, irinotecan or topotecan) unless they develop or have progressive or persistent leptomeningeal metastases while on these agents; systemic therapy (including investigational agents and small-molecule kinase inhibitors) within 14 days or 5 half-lives, whichever is shorter, prior first dose of study drug (¹⁸⁶RNL); nitrosoureas or mitomycin C within 42 days, or metronomic/protracted low-dose chemotherapy within 14 days, or other cytotoxic chemotherapy within 28 days, prior to first dose of study drug (¹⁸⁶RNL); and impaired CSF Flow Study performed on Day −4 to Day −2 based on study imaging and as determined by the investigator.

### Example 11: Treatment of Leptomeningeal Metastases

The Maximum Tolerated Dose/Maximum Feasible Dose, Safety, & Efficacy of Single Dose Rhenium-186 Nanoliposome (186RNL) Administered via the Intraventricular Route for Leptomeningeal Metastasis will be determined. The Primary Objectives are to characterize the safety & tolerability of a single dose of 186RNL by the intraventricular route & to identify a maximum tolerated dose (MTD) and/or maximum feasible dose (MFD) and develop a collaboration for CSF Biomarker Analysis. The Secondary Objectives are to characterize the pharmacokinetic & dosimetry profile of a single dose of 186RNL when administered intraventricularly via Ommaya reservoir, develop a multiple dosing strategy of 186RNL for subsequent clinical trials, determine the overall response rate (ORR) defined as the proportion of all evaluable patients achieving a response as the best overall response at the time of progression, determine the duration or response (DoR) defined as the time from first response to LM progression, determine progression free survival (PFS) defined as the time from first treatment to date of LM progression or death from any cause, and determine the overall survival (OS) definined as the time from first treatment to date of death. Primary Endpoints are to determine incidence & severity of adverse events (AE) & serious adverse events (SAE) and incidence of dose limiting toxicities (DLT).

### Example 12: 186RNL in Leptomeningeal Cancer

Leptomeningeal cancer, also known as carcinomatosis, is a cancer that starts in one part of the body spreads to the leptomeningeal lining of the brain and spinal cord surrounding the cerebrospinal fluid (CSF) space. 100 nm nanoliposomes will be administered. The nanoliposomes circulate freely throughout the CSF, migrate to meningeal surfaces where LMC is located, have an extended half-life—several weeks vs. hours with unencapsulated drugs, and are safe and effective in preclinical models.

### Example 13. Dose Escalation Scheme for Leptomeningeal Metastases

The infused dose will be progressively increased from 6.6 mCi to 82.5 mCi and the volume of infusate will be 5 mL. The study design is summarized in Table 5 below.

TABLE 5
Dose escalation scheme

| | | | | Theoretical | |
|---|---|---|---|---|---|
| | | | | Maximal | |
| | | Total RNL | | Absorbed | |
| | Volume | Activity | Concentration | Dose in CSF | Increase |
| Cohort | (mL) | (mCi) | mCi/ml | (Gy)* | (%) |
| 1 | 5 | 6.6 | 1.32 | 50 | N/A |
| 2 | 5 | 13.2 | 2.64 | 100 | 100 |
| 3 | 5 | 26.4 | 5.28 | 200 | 100 |
| 4 | 5 | 42.9 | 8.58 | 325 | 62.5 |
| 5 | 5 | 60.1 | 12.02 | 455 | 40 |
| 6 | 5 | 75.1 | 15.01 | 569 | 25 |
| 7 | 5 | 82.5 | 16.52 | 626 | 10 |

*Theoretical Maximal Absorbed Dose is the radiation absorbed dose in the condition of no clearance from the CSF volume after RNL infusion (100% sustained retention in CSF volume).

### Example 13: Additional Patient Data for Treating GBM

Twenty-three patients were treated of which 30.4% had failed treatment with bevacizumab. The infused dose was progressively increased from 1.0 mCi to 31.2 mCi and the volume of infusate was increased from 0.66 mL to 12.3 mL. The study design and overall survival of each patient is summarized in Table 6 below. Overall Survival is as of day of table generation.

TABLE 6
Phase 1 Clinical Trial Design

| | | Volume | | | | | | | |
|---|---|---|---|---|---|---|---|---|---|
| | | of | | | Maximum | Prior | | Overall | |
| | Dose | Infusate | Conc. | | Flow Rate | Treatment | Bevacizumab | Survival | |
| Patient | (mCi) | [Vi] (mL) | (mCi/mL) | Catheters | (μL/min) | Cycles | Failure | (days) | Alive |
| 1 | 1.0 | 0.66 | 1.5 | 1 | 5 | 1 | N | 909 | N |
| 2 | 1.0 | 0.66 | 1.5 | 1 | 5 | 1 | N | 1065 | N |
| 3 | 1.0 | 0.66 | 1.5 | 1 | 5 | 2 | N | 264 | N |
| 4 | 2.0 | 1.32 | 1.5 | 1 | 5 | 3 | Y | 51 | N |
| 5 | 2.0 | 1.32 | 1.5 | 1 | 5 | 1 | N | 331 | N |
| 6 | 2.0 | 1.32 | 1.5 | 1 | 5 | 2 | Y | 46 | N |
| 7 | 4.0 | 2.64 | 1.5 | 1 | 5 | 2 | Y | 318 | N |
| 8 | 4.0 | 2.64 | 1.5 | 1 | 5 | 2 | Y | 175 | N |
| 9 | 4.0 | 2.64 | 1.5 | 1 | 5 | 2 | Y | 125 | N |
| 10 | 8.0 | 5.28 | 1.5 | 1 | 10 | 3 | Y | 145 | N |
| 11 | 8.0 | 5.28 | 1.5 | 2 | 10 | 2 | Y | 157 | N |
| 12 | 8.0 | 5.28 | 1.5 | 2 | 10 | 1 | N | 1185 | N |
| 13 | 13.4 | 5.28 | 2.5 | 2 | 10 | 3 | N | 1001 | Y |
| 14 | 13.4 | 5.28 | 2.5 | 3 | 15 | 2 | N | 246 | N |
| 15 | 13.4 | 5.28 | 2.5 | 3 | 15 | 1 | N | 291 | N |
| 16 | 22.3 | 8.8 | 2.5 | 2 | 15 | 1 | N | 140 | N |
| 17 | 22.3 | 8.8 | 2.5 | 4 | 15 | 1 | N | 370 | N |
| 18 | 22.3 | 8.8 | 2.5 | 3 | 15 | 1 | N | 630 | Y |
| 19 | 22.3 | 8.8 | 2.5 | 4 | 20 | 1 | N | 185 | N |
| 20 | 22.3 | 8.8 | 2.5 | 2 | 20 | 2 | N | 504 | Y |
| 21 | 22.3 | 8.8 | 2.5 | 2 | 20 | 1 | N | 370 | N |
| 22 | 31.2 | 12.3 | 2.5 | 3 | 20 | 1 | N | 219 | N |
| 23 | 31.2 | 12.3 | 2.5 | 3 | 20 | 2 | N | 161 | N |

The median survival duration in patients that had failed bevacizumab treatment (n=7) was 145 days. The median survival duration in bevacizumab-naïve patients (n=16) was 350.5 days, with 3 patients still alive at the conclusion of the study.

### Example 16: ReSPECT-LM Clinical Update

A multi-center Phase 1 study is being performed with the primary objectives of determining the maximum tolerated dose/maximum feasible dose, safety, and efficacy of single dose rhenium (186Re) obisbemeda administered via intraventricularly for leptomeningeal metastases (LM). Secondary objectives are pharmokinetic and dosimetry profile of a single dose of 186RNL when administered intraventricularly via an Ommaya reservoir, develop a multiple dosing strategy of 186RNL for subsequent clinical trials, determine the overall response rate (ORR), duration of response (DoR), progression-free survival, and overall survival (OS). Primary endpoints are the incidence and severity of adverse events (AE) and serious adverse events (SAE) and incidence of dose limiting toxicities (DLT). FIG. 32 shows leptomeningeal administration of rhenium (186Re) obisbemeda targeted to the CSF and rapid diffusion. FIG. 33 shows updates on the ReSPECT-LM Clinical Trial.

### Example 17: Animal Studies with RNL and Immunotherapy Drugs

Animal studies will be performed administering RNL and one or more immunotherapy treatments. Immunotherapy treatments that can be administered with RNL include, but are not limited to, monoclonal antibodies and immune checkpoint inhibitors, PARP inhibitors, non-specific immunotherapies, oncolytic virus therapy, T-cell therapy, and cancer vaccines. T-cell therapy includes, but is not limited to CAR T-cell therapy and tumor-infiltrating lymphocytes. The immunotherapy treatments can be co-administered with the RNL or administered before or after the RNL. Immunotherapy treatments that can be administered with RNL also include, but are not limited to, the drugs listed in Table 7. Various doses and treatment schedules will be tested. The immunotherapy treatments will be delivered systemically or into the cerebrospinal fluid (CSF).

TABLE 7
Immunotherapy Drugs

| Drug name | Brand Name |
|---|---|
| Olaparib | Lynparza |
| Rucaparib | Rubraca |
| Niraparib | Zejula |
| T-VEC | Imlygic |
| Pomalidomide | Imnovid |
| Thalidomide | Thalidomid |
| Lenalidomide | Revlimid |
| Brexucabtagene autoleucel | Tecartus |
| Ado-trastuzumab emtansine | Kadcyla |
| Aldesleukin | Proleukin |
| Amivantamab-vmjw | Rybrevant |
| Atezolizumab | Tecentriq |
| Avelumab | Bavencio |
| Axicabtagene ciloleucel | Yescarta |
| Belantamab Mafodotin-blmf | Blenrep |
| Bevacizumab | Avastin |
| Bevacizumab-awwb | Mvasi |
| Bevacizumab-bvzr | Zirabev |
| Blinatumomab | Blincyto |
| Brentuximab Vedotin | Adcetris |
| Cemiplimab-rwlc | Libtayo |
| Cetuximab | Erbitux |
| Ciltacabtagene autoleucel | Carvykti |
| Daratumumab | Darzalex |
| Daratumumab and hyaluronidase-fihj | Darzalex Faspro |
| Dostarlimab-gxly | Jemperli |
| Durvalumab | Imfinzi |
| Elotuzumab | Empliciti |
| Enfortumab Vedotin-ejfv | Padcev |
| Fam-trastuzumab deruxtecan-nxki | Enhertu |
| Gemtuzumab Ozogamicin | Mylotarg |
| Ibritumomab Tiuxetan | Zevalin |
| Idecabtagene Vicleucel | Abecma |
| Inotuzumab Ozogamicin | Besponsa |
| Interferon alfa-2b | IntronA |
| Ipilimumab | Yervoy |
| Isatuximab-irfc | Sarclisa |
| Lisocabtagene Maraleucel | Breyanzi |
| Loncastuximab tesirine-lpy | Zynlonta |
| Margetuximab-cmkb | Margenza |
| Mirvetuximab soravtansine-gynx | Elahere |
| Mogamulizumab | Poteligeo |
| Moxetumomab Pasudotox-tdfk | Lumoxiti |
| Naxitamab | Danyelza |
| Necitumumab | Portrazza |
| Nivolumab | Opdivo |
| Nivolumab and relatlimab-rmbw | Opdualag |
| Obinutuzumab | Gazyvaro |
| Obinutuzumab | Gazyva |
| Ofatumumab | Arzerra |
| Panitumumab | Vectibix |
| Peginterferon alfa-2b | Sylatron |
| Pembrolizumab | Keytruda |
| Pertuzumab | Perjeta |
| Pertuzumab, Trastuzumab, | Phesgo |
| hyaluronidase-zzxf combination | |
| Polatuzumab Vedotin-Piiq | Polivy |
| Ramucirumab | Cyramza |
| Rituximab | Blitzima |
| Rituximab | Riximyo |
| Rituximab | Rituxan |
| Rituximab | MabThera |
| Rituximab | Rixathon |
| Rituximab-abbs | Truxima |
| Sacituzumab Govitecan-hziy | Trodelvy |
| Siltuximab | Sylvant |
| Sipuleucel-T | Provenge |
| Tafasitamab | Minjuvi |
| Tafasitamab-cxix | Monjuvi |
| Tagraxofusp-erzs | Elzonris |
| Talimogene laherparepvec | Imlygic |
| Tebentafusp-tebn | Kimmtrak |
| Teclistamab-cpyv | Tecvayli |
| Tisagenlecleucel | Kymriah |
| Tisotumab Vedotin-tftv | Tivdak |
| Trastuzumab | Zercepac |
| Trastuzumab | Kanjinti |
| Trastuzumab | Ogivri |
| Trastuzumab | Ontruzant |
| Trastuzumab | Herzuma |
| Trastuzumab | Trazimera |
| Trastuzumab | Herceptin |
| Trastuzumab and Hyaluronidase | Herceptin Hylecta |
| Tremelimumab-actl | Imjudo |
| ziv-Aflibercept | Zaltrap |

Although the technology herein has been described with reference to embodiments, it is to be understood that these embodiments are merely illustrative of the principles and applications of the present technology. It is therefore to be understood that numerous modifications may be made to the illustrative embodiments and that other arrangements may be devised without departing from the spirit and scope of the present technology as defined by the appended claims.

What is claimed is:

1. A method of treating leptomeningeal metastases in a subject in need thereof, comprising administering intraventricularly a therapeutically effective amount of a radiolabeled liposome comprising a liposome and a compound of Formula I: 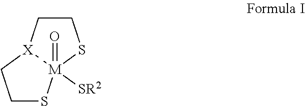
or a pharmaceutically acceptable salt thereof, wherein:
M is ^{99m}Tc, ¹⁸⁶Re, ¹⁸⁸Re, or a combination thereof;
X is NR¹;
R¹ is CH₂CH₂NEt₂; and
R² is CH₂CH₂N(CH₂CH₂SH)(CH₂CH₂NEt₂).

2. The method of claim 1 wherein M is ¹⁸⁶Re.

3. The method of claim 1 wherein the compound is incorporated or attached to the liposome.

4. The method of claim 1 wherein the liposome comprises a phospholipid and a cholesterol or a cholesterol analogue.

5. The method of claim 4 wherein the liposome comprises distearoyl phosphatidylcholine.

6. The method of claim 1 wherein the liposome further comprises a chemotherapeutic agent, an antibiotic agent, or a treatment molecule, wherein the chemotherapeutic agent, the antibiotic agent, or the treatment molecule is incorporated or attached to the liposome.

7. The method of claim 1 wherein the leptomeningeal metastases originated from lung cancer, breast cancer, or melanoma.

8. The method of claim 1 wherein the radiolabeled liposome is administered as an infusate comprising the radiolabeled liposome.

9. The method of claim 8 wherein the radiolabeled liposome is administered as an infusate via an Ommaya reservoir.

10. The method of claim 8 wherein the infusate is administered with a maximum flow rate from about 1 μL min⁻¹ to about 50 μL min⁻¹.

11. The method of claim 8 wherein an amount of radioactivity delivered by the infusate is from about 0.1 mCi to about 50 mCi.

12. The method of claim 8 wherein the volume of the infusate is from about 0.1 mL to about 25 mL.

13. The method of claim 12 wherein the volume of the infusate is about 5.0 mL.

14. The method of claim 8 wherein the amount of radioactivity delivered by the radiolabeled liposome per volume of the infusate is from about 0.1 mCi mL⁻¹ to about 50 mCi mL⁻¹.

15. The method of claim 14 wherein the amount of radioactivity delivered by the radiolabeled liposome per volume of the infusate is from about 1.32 mCi mL⁻¹ to about 5.28 mCi mL⁻¹.

16. The method of claim 8 wherein the infusate is administered with an exit velocity from about 0.001 mm/min to about 1.0 mm/min.

17. The method of claim 8 further comprising an absorbed dose from about 5 Gy to about 100 Gy.

18. The method of claim 8 further comprising an absorbed dose from about 100 Gy to about 800 Gy.

19. The method of claim 8 further comprising an absorbed dose from about 50 Gy to about 200 Gy.

20. The method of claim 1 wherein the method further comprises imaging the radiolabeled liposome concomitant with or subsequent to administration.
